(19) United States
(12) Patent Application Publication
(10) Pub. No.: US 2006/0275294 A1
(43) Pub. Date: Dec. 7, 2006

(54) METHOD OF PREVENTION AND TREATMENT OF AGING, AGE-RELATED DISORDERS AND/OR AGE-RELATED MANIFESTATIONS INCLUDING ATHEROSCLEROSIS, PERIPHERAL VASCULAR DISEASE, CORONARY ARTERY DISEASE, OSTEOPOROSIS, ARTHRITIS, TYPE 2 DIABETES, DEMENTIA, ALZHEIMERS DISEASE AND CANCER

(76) Inventor: Osemwota Sota Omoigui, Tarzana, CA (US)

Correspondence Address: BLACK LOWE & GRAHAM, PLLC, 701 FIFTH AVENUE, SUITE 4800, SEATTLE, WA, 98104, US

(21) Appl. No.: 11/279,239

(22) Filed: Apr. 10, 2006

Related U.S. Application Data
(63) Continuation-in-part of application No. 11/268,609, filed on Nov. 8, 2005.
Application No. 11/268,609 is a continuation-in-part of application No. 11/122,030, filed on May 5, 2005.
Application No. 11/122,030 is a continuation-in-part of application No. 10/961,037, filed on Oct. 12, 2004.
(63) Continuation-in-part of application No. 11/058,371, filed on Feb. 16, 2005.
Application No. 11/058,371 is a continuation-in-part of application No. 10/224,743, filed on Aug. 22, 2002, now abandoned.

(51) Int. Cl. A61K 39/395; A61K 31/7048; A61K 31/675; A61K 31/663; A61K 31/401; A61K 31/366; A61K 31/353; A61K 31/22; A61K 36/73; A61K 36/06

(52) U.S. Cl. 424/145.1; 424/195.16; 514/102; 514/423; 514/460; 514/548; 514/89; 514/27; 514/456; 514/776; 514/757; 424/777; 424/765

(57) ABSTRACT

This invention relates to a method for prevention and treatment of aging, age-related disorders and/or age-related manifestations including atherosclerosis, peripheral vascular disease, coronary artery disease, osteoporosis, type 2 diabetes, dementia and some forms of arthritis and cancer in a subject comprising administering to said subject, separately, sequentially or simultaneously a therapeutically effective dosage of each component or combination of statins, bisphosphonates, cholesterol lowering agents or techniques, interleukin-6 inhibitor/antibody, interleukin-6 receptor inhibitor/antibody, interleukin-6 antisense oligonucleotide (ASON), gp130 protein inhibitor/antibody, tyrosine kinases inhibitors/antibodies, serine/threonine kinases inhibitors/antibodies, mitogen-activated protein (MAP) kinase inhibitors/antibodies, phosphatidylinositol 3-kinase (PI3K) inhibitors/antibodies, Nuclear factor κB (NF-κB) inhibitors/antibodies, IκB kinase (IKK) inhibitors/antibodies, activator protein-1 (AP-1) inhibitors/antibodies, STAT transcription factors inhibitors/antibodies, altered IL-6, partial peptides of IL-6 or IL-6 receptor, or SOCS (suppressors of cytokine signaling) protein, or a functional fragment thereof, administered separately, in sequence or simultaneously. Inhibition of the signal transduction pathway for Interleukin 6 mediated inflammation is key to the prevention and treatment of atherosclerosis, peripheral vascular disease, coronary artery disease, aging, age-related disorders and/or age-related manifestations including osteoporosis, type 2 diabetes, dementia and some forms of arthritis and tumors. Inhibition of Interleukin 6 mediated inflammation may be achieved indirectly through regulation of endogenous cholesterol synthesis and isoprenoid depletion or by direct inhibition of the signal transduction pathway utilizing interleukin-6 inhibitor/antibody, interleukin-6 receptor inhibitor/antibody, interleukin-6 antisense oligonucleotide (ASON), gp130 protein inhibitor/antibody, tyrosine kinases inhibitors/antibodies, serine/threonine kinases inhibitors/antibodies, mitogen-activated protein (MAP) kinase inhibitors/antibodies, phosphatidylinositol 3-kinase (PI3K) inhibitors/antibodies, Nuclear factor κB (NF-κB) inhibitors/antibodies, IκB kinase (IKK) inhibitors/antibodies, activator protein-1 (AP-1) inhibitors/antibodies, STAT transcription factors inhibitors/antibodies, altered IL-6, partial peptides of IL-6 or IL-6 receptor, or SOCS (suppressors of cytokine signaling) protein, or a functional fragment thereof. Said method for prevention and treatment of said disorders is based on inhibition of Interleukin-6 inflammation through regulation of cholesterol metabolism, isoprenoid depletion and/or inhibition of the signal transduction pathway

### PRIORITY CLAIM

This application is a continuation-in-part of and claims priority to U.S. patent application Ser. No. 11/268,609 filed Nov. 8, 2005 which is a continuation-in-part of and claims priority to U.S. patent application Ser. No. 11/122,030 filed May 5, 2005, which is a continuation-in-part of and claims priority to U.S. patent application Ser. No. 10/961,037 filed Oct. 12, 2004. This application is also a continuation-in-part of and claims priority to U.S. patent application Ser. No. 11/058,371 filed Feb. 16, 2005, which application is a continuation-in-part of U.S. patent application Ser. No. 10/224,743 filed Aug. 22, 2002. All of the above applications are hereby incorporated by reference in their entirety as if fully set forth herein.

### COPYRIGHT NOTICE

This disclosure is protected under United States and International Copyright Laws. © 2002-2006 Osemwota Sota Omoigui. All Rights Reserved. A portion of the disclosure of this patent document contains material which is subject to copyright protection. The copyright owner has no objection to the facsimile reproduction by anyone of the patent document or the patent disclosure after formal publication by the USPTO, as it appears in the Patent and Trademark Office patent file or records, but otherwise reserves all copyright rights whatsoever.

### FIELD OF THE INVENTION

This invention relates to a method of prevention and treatment of aging, age-related disorders and/or age-related manifestations including Atherosclerosis, Peripheral Vascular Disease, Coronary Artery Disease, Osteoporosis, Type 2 Diabetes, Dementia, Alzheimer's disease and some forms of Arthritis and Cancer, by inhibition of Interleukin 6 mediated inflammation. Inhibition of Interleukin 6 mediated inflammation may be achieved indirectly through regulation of endogenous cholesterol synthesis and isoprenoid depletion or by direct inhibition of the signal transduction pathway utilizing interleukin-6 inhibitor/antibody, interleukin-6 receptor inhibitor/antibody, interleukin-6 antisense oligonucleotide (ASON), gp130 protein inhibitor/antibody, tyrosine kinases inhibitors/antibodies, serine/threonine kinases inhibitors/antibodies, mitogen-activated protein (MAP) kinase inhibitors/antibodies, phosphatidylinositol 3-kinase (PI3K) inhibitors/antibodies, Nuclear factor κB (NF-κB) inhibitors/antibodies, IκB kinase (IKK) inhibitors/antibodies, activator protein-1 (AP-1) inhibitors/antibodies, STAT transcription factors inhibitors/antibodies, altered IL-6, partial peptides of IL-6 or IL-6 receptor, or SOCS (suppressors of cytokine signaling) protein, PPAR gamma and/or PPAR beta/delta activators/ligands or a functional fragment thereof. Compositions may be used for human and veterinary use, and may be, for example, in a form of a food, a dietary supplement or a pharmaceutical.

Interleukin 6 mediated inflammation is the common causative origin for aging, age-related disorders and/or age-related manifestations including Atherosclerosis, Peripheral Vascular Disease, Coronary Artery Disease, Osteoporosis, Type 2 Diabetes, Dementia, Alzheimer's disease and some forms of Arthritis and Cancer.

### BACKGROUND OF THE INVENTION

The current theories and treatment options for aging, age-related disorders and/or age-related manifestations including Atherosclerosis, Peripheral Vascular Disease, Coronary Artery Disease, Osteoporosis, Type 2 Diabetes, Dementia and Alzheimer's disease and some forms of Arthritis and Cancer are fragmented and not satisfactory. There is currently no unifying theory that links Interleukin-6 mediated inflammation as the common causative origin for aging, age-related disorders and/or age-related manifestations and all the above diseases. As such current strategies for each disease entails different medications and therapeutic procedures such as statins, aspirin, beta blockers, ACE inhibitors and angioplasty for atherosclerosis and coronary heart diseasei, statins and thrombolytics for peripheral vascular disease, oral hypoglycemics for Type 2 diabetes, bisphosphonates and calcitonin for osteoporosis, and Acetylcholinesterase inhibitors e.g. rivastigmine, donepezil and galanthamine for dementia and Alzheimer's disease. The prior theories attribute the beneficial health effects of plants and vegetables to antioxidant activity. The prior theories do not provide the mechanism of action of plant derived and synthesized polyphenolic compounds in the biochemical pathway that links Interleukin-6 mediated inflammation as the common causative origin for aging, age-related disorders and/or age-related manifestations.

### SUMMARY OF THE INVENTION

The present invention provides a method for the prevention and treatment of aging, age-related disorders and/or age-related manifestations including Atherosclerosis, Peripheral Vascular Disease, Coronary Artery Disease, Osteoporosis, Type 2 Diabetes, Dementia and Alzheimer's disease and some forms of Arthritis and Cancer, in a human or other animal subject. Inhibition of the signal transduction pathway for Interleukin 6 mediated inflammation is key to the prevention and treatment of aging, age-related disorders and/or age-related manifestations including atherosclerosis, peripheral vascular disease, coronary artery disease, osteoporosis, type 2 diabetes, dementia and some forms of arthritis and tumors. Inhibition of Interleukin 6 mediated inflammation may be achieved indirectly through regulation of endogenous cholesterol synthesis and isoprenoid depletion or by direct inhibition of the signal transduction pathway utilizing interleukin-6 inhibitor/antibody, interleukin-6 receptor inhibitor/antibody, interleukin-6 antisense oligonucleotide (ASON), gp130 protein inhibitor/antibody, tyrosine kinases inhibitors/antibodies, serine/threonine kinases inhibitors/antibodies, mitogen-activated protein (MAP) kinase inhibitors/antibodies, phosphatidylinositol 3-kinase (PI3K) inhibitors/antibodies, Nuclear factor κB (NF-κB) inhibitors/antibodies, IκB kinase (IKK) inhibitors/antibodies, activator protein-1 (AP-1) inhibitors/antibodies, STAT transcription factors inhibitors/antibodies, altered IL-6, partial peptides of IL-6 or IL-6 receptor, or SOCS (suppressors of cytokine signaling) protein, PPAR gamma and/or PPAR beta/delta activators/ligands or a functional fragment thereof. Compositions may be used for human and veterinary use, and may be, for example, in a form of a food, a dietary supplement or a pharmaceutical.

### BRIEF DESCRIPTION OF THE DRAWINGS

FIG. 1. Mevalonate Synthesis.

FIG. 2. Isoprenoid Synthesis.

### DETAILED DESCRIPTION OF THE PREFERRED EMBODIMENT

In 400 B.C., Hippocrates recognized the relationship between health and food. He said: “Let food be your medicine and medicine be your food”. In 1513, Spanish explorer Juan Ponce de Leon discovered Florida while searching for the Fountain of Youth, a mythical spring said to restore youth. Ponce de Leon died trying to find those waters. He should have been looking instead for the Flora of Youth and inhibitors of Interleukin 6 mediated inflammation.

Aging is associated with several disorders including Atherosclerosis, Peripheral Vascular Disease, Coronary Artery Disease, Osteoporosis, Type 2 Diabetes, Dementia and Alzheimer's disease and some forms of Arthritis and Cancer. It is our theory that inhibition of Interleukin 6 mediated inflammation is key to the prevention and treatment of aging, age-related disorders and/or age-related manifestations.

ATHEROSCLEROSIS Cardiovascular disease (CVD) is the leading cause of death and disability in developed nations and is increasing rapidly in the developing world. By the year 2020, it is estimated that CVD will surpass infectious diseases as the world's leading cause of death and disability. Atherosclerotic vascular disease (ASVD), which encompasses coronary heart disease, cerebrovascular disease, and peripheral arterial disease, is responsible for the majority of cases of CVD in both developing and developed countriesii. Atherosclerosis, a progressive disease characterized by the accumulation of lipids and fibrous elements in the arteries, constitutes the single most important contributor to this growing burden of cardiovascular disease. The link between lipid metabolism and atherosclerosis dominated the thinking until the 1980siii. Over the last fifteen years, however, a prominent role for inflammation in the pathogenesis of atherosclerosis has been establishediv. Now atherosclerosis is considered as an inflammation-mediated disease driven by complex interactions between leukocytes, platelets and cells of the vessel wall. Endothelial injury is the first and crucial step in the pathogenesis of atherosclerosis. A plethora of genetically determined and epigenetic factors, such as oxidized low-density lipoprotein (LDL), free radicals (e.g., due to cigarette smoking), hypertension, diabetes mellitus, elevated plasma homocysteine, infectious microorganisms, autoimmune reactions, and combinations thereof, have been identified as etiological principles. Endothelial injury triggers inflammation with increased adhesiveness and activation of leukocytes (mainly monocytes) and platelets, which is accompanied by the production of cytokines, chemokines, vasoactive molecules and growth factors.

The hallmark of the early atherosclerotic lesion is the Cholesterol ester-laden (CE-laden) macrophage foam cellv. Progressive “free” cholesterol (FC) loading of lesional macrophages leads to a series of phospholipid-related adaptive responses. These adaptive responses eventually fail, leading to macrophage death. Macrophage death by either necrosis or apoptosis leads to lesional necrosis, release of cellular proteases, inflammatory cytokines, and prothrombotic molecules, which could contribute to plaque instability, plaque rupture, and acute thrombotic vascular occlusionvi. Indeed, necrotic areas of advanced atherosclerotic lesions are known to be associated with death of macrophages, and ruptured plaques from human lesions have been shown to be enriched in apoptotic macrophages. The presence of apoptotic and necrotic macrophages in atherosclerotic lesions has been well documented in many human and animal studiesvii viii

Currently, the inflammatory mediators implicated in the pathogenesis of atherosclerosis include cytokines, chemokines, vasoactive molecules and growth factors. The anti-inflammatory effects of statins are attributed to multifaceted mechanisms including inhibition of cell cycle progression, induction of apoptosis, reduction of cyclooxygenase-2 activity and an enhancement of angiogenesis. At the center of these mechanisms stands the ability to inhibit G protein prenylation through a reduction of farnesylation and geranylgeranylationix.

In order to advance the current theories and thinkingx, and clarify the relationship between these common illnesses, we submit our theory of the precise biochemical pathway, between cholesterol synthesis and inflammation, and between inflammation and aging, age-related disorders and/or age-related manifestations including Atherosclerosis, Peripheral Vascular Disease, Coronary Artery Disease, Osteoporosis, Type 2 Diabetes, Dementia and Alzheimer's disease and some forms of Arthritis and Cancer. By elaborating this biochemical pathway, we will delineate the precise mechanism of the pleiotropic effects of statins, bisphosphonate drugs and polyphenolic compounds. The common mechanism of action and common pleiotropic effects of the statins, bisphosphonate drugs and polyphenolic compounds studies in addition to our identification of the unique activity of the Interleukin 6 cytokine among all the vast mediators of inflammation and the inflammatory response enabled us to reverse engineer this biochemical pathway. Each component of our theory is supported and validated by numerous research.

ACUTE PHASE RESPONSE The acute phase response occurs prior to antibody-mediated immunological defense. It occurs in response to an inflammatory response brought on by injury and trauma, neoplasm, or disordered immunological activity. A local reaction at the site of injury or infection leads to an activation of cytokines (specifically, IL-6, IL-1, TNF-Alpha, and interferons) that triggers a systemic response consisting of leukocytosis; increases in glucocorticoid production; increases in erythrocyte sedimentation rates, fever, activation of complement and clotting cascades; decreases in serum zinc and iron; and an increase in plasma levels of acute phase proteins, C-reactive protein (CRP), serum amyloid A, fibrinogen, and other proteinsxi

Levels of cytokines involved in the acute phase response—TNF-Alpha, IL-1, IL-6, and fibrinogen—have been shown to be elevated in cases of unstable angina related to aneurysmxii xiii xiv and have been positively correlated with the risk of primary and recurrent myocardial infarction and deathxv xvi xvii. The risk associated with these elevated levels remains constant even when the data is adjusted for other major risk factors: blood pressure, total and HDL cholesterol, body mass index, diabetes, alcohol use, family history, and exercise frequency15. Elevated levels of highly sensitive C-reactive protein (hs-CRP) have been related to increased risk of cardiovascular disease, myocardial infarction, and coronary artery disease (CAD) deaths among individuals with angina pectorisxviii xix xx. Assayed levels of hs-CRP can increase 100 times over normal levels within 24-48 hours after an acute inflammatory stimulus. However, in long term prospective studies inter-individual variations in hs-CRP levels may occur over long periods of time, in the absence of trauma or acute infectionxxi Elevated levels of hs-CRP have shown a doubling of risk both for ischemic stroke in hypertensive men and women14 xxii and for peripheral artery diseasexxiii

Recent studies are now demonstrating that IL-6 and TNF-alpha are stronger predictors of cardiovascular disease than C-reactive protein. In the Health, Aging and Body Composition studyxxiv, done at the Wake Forest University School of Medicine, the researchers tracked the medical history of the 2,225 participants for an average of 42 months after measuring their blood levels of C-reactive protein, IL-6 and TNF-alpha. People with the highest IL-6 levels were two to five times more likely to have a heart attack, stroke or other cardiovascular episode than those with the lowest levels. High blood levels of TNF-alpha increased the risk of heart disease by 79 percent and of heart failure by 121 percent. High levels of C-reactive protein increased the risk of heart failure by 160 percent compared to those with low levels, but they did not significantly raise the risk of a first stroke or heart attack.

As expected, the incidence of cardiovascular disease was high for people with the conventional risk factors—smoking, high blood pressure, high cholesterol and the like. But for participants free of those risk factors, the inflammation-related molecules were better predictors of heart disease.

INTERLEUKIN 6 Cytokines play an important role in the communication between cells of multicellular organisms. As intercellular mediators acting in nanomolar to picomolar concentrations they regulate survival, growth, differentiation and effector functions of cells. They are key players in the regulation of the immune response. Cytokines act on many different target cells (pleiotropism) and frequently affect the action of other cytokines in an additive, synergistic or antagonistic manner9. The Interleukin-6 family of cytokines, signaling through the common receptor subunit (glycoprotein) subsequently activates signal transducers and activators of transcription (STAT3), mitogen-activated protein kinase (MAPK), and phosphatidylinositol 3-kinase (PI3K)xxv. The interleukin-6 (IL6) family comprises interleukin (IL)-6, IL-11, leukemia inhibitory factor, oncostatin M, ciliary neurotrophic factor and cardiotrophin-1. Among its many functions, IL-6 plays an active role in inflammation, immunology, bone metabolism, reproduction, arthritis, neoplasia, and aging. IL-6 expression is regulated by a variety of factors, including steroidal hormones, at both the transcriptional and post-transcriptional levels. Elevated levels of IL-6 are associated with the highest risks for subclinical cardiovascular disease as well as for clinical cardiovascular disease in older men and womenxxvi. Elevated levels of IL-6 are associated with a 34 percent increased likelihood of cognitive decline in older men and womenxxvii. Interleukin-6 mediated inflammation contributes to bone resorption and osteoporosis by stimulating osteoclastogenesis and osteoclast activityxxviii xxix xxx. Elevated levels of Interleukin-6 have been observed in conditions of rapid skeletal turnover and hypercalcemia as in Paget's disease and multiple myeloma. In multiple myeloma, radiologic examinations reveals osteolytic lesion with the most common finding being diffuse osteopeniaxxxi. Adhesion of multiple myeloma cells to stromal cells triggers IL-6 secretion by the stromal cells. The increased osteoclastic activity results in osteoporosis, painful osteolytic lesions and hypercalcemia characteristic of multiple myelomaxxxii. In their youth, women are protected from osteoporosis because of the presence of sufficient levels of estrogen. Estrogen blocks the osteoclast's synthesis of Interleukin 6 and may also antagonize the Interleukin 6 receptors. Decline in estrogen production is often associated with osteopenia or osteoporosis in postmenopausal womenxxxiii xxxiv xxxv xxxvi. Inflammatory joint disease, particularly rheumatoid arthritisxxxvii, is associated with bone resorption and increased synovial fluid levels of IL-6xxxviii. Interleukin (IL)-6 production is considerably enhanced and associated with bone destruction in Staphylococcus aureus and mycobacterial arthritis, osteitis or osteomyelitisxxxix xl xli. During times of stress or depression, IL-6 levels are increased. In a study of older adults undergoing a chronic stressor (men and women who were caregiving for a spouse with dementia), Caregivers' average rate of increase in IL-6 was about four times as large as that of non-caregiversxlii xliii.

IL-6 transmits its biological signal through two proteins on the cell. One of them is IL-6 receptor (IL-6R), an IL-6-specific binding molecule with a molecular weight of about 80 kD. The other is a membrane-bound protein gp130 having a molecular weight of about 130 kD that is involved in non-ligand-binding signal transduction. IL-6 receptor exists not only in the membrane-bound form with transmembrane domain expressed on the cell surface but also as a soluble IL-6 receptor consisting mainly of the extracellular region. IL-6 and IL-6 receptor form the IL-6/IL-6 receptor complex, which after binding to gp130 transmits its biological signal to the cell. The important participants in the Interleukin-6 signaling pathway include the Janus kinases (JAKs) Jak1, Jak2 and Tyk2, the signal transducers and activators of transcription STAT1 and STAT3, the tyrosine phosphatase SHP2 [SH2 (Src homology 2) domain-containing tyrosine phosphatase] and transcription factor NF-κB.

PROTEIN KINASES Protein kinases are a class of allosteric enzymes that possess a catalytic subunit which transfers a phosphate from ATP to one or more amino acid residues (as serine, threonine, or tyrosine) in a protein's side chain resulting in a conformational change affecting protein function, that play a role in regulating intracellular processes. JAK kinases; (abbreviation for janus-activated kinase) is the name given to a family of non-receptor protein tyrosine kinases, comprising JAK1 (Janus kinase-1), JAK2 (Janus kinase-2), Tyk2 (non-receptor protein tyrosine kinase-2), which are widely expressed and JAK3 (Janus kinase-3) which is mainly found in cells of haematopoietic origin. STATS comprise a family of seven transcription factors that are activated by a variety of cytokines, hormones and growth factors.xliv. Engagement of cell surface Interleukin-6 receptors activates the Janus kinase (JAK) family of tyrosine kinases, which in turn phosphorylate the cytoplasmic part of gp130, thereby creating docking sites for STAT factors STAT1 and STAT3xlv xlvi. Activated STATs dimerize upon activation by JAKs and translocate to the nucleus where they bind specific DNA response elements and regulate the expression of certain genes. Following gp130 dimerization, IL-6 activates multiple signaling pathways (Ras dependent MAP Kinase cascade, STAT1-STAT3 heterodimer pathway, and STAT3 homodimer pathway)xlvii xlviii xlix STAT3 is constitutively activated in bone marrow mononuclear cells in patients with myeloma. High levels of activated STAT3 are found in the myeloma cell line U266 known to produce and utilize IL-6 for survivall.

A family of cytokine-inducible proteins inhibits the Jak-STAT signaling cascade providing an intracellular negative feedback regulation of cytokine-induced signal activation. These proteins have been variously termed suppressors of cytokine signaling (SOCS)li, STAT-induced STAT inhibitors (SSI)lii, cytokine-inducible SH2 containing protein (CIS), and Jak binding protein (JAB). The SOCS-protein family currently consists of CIS and SOCS-1 through 7. SOCS-protein expression is stimulated by various cytokines in a tissue specific manner. The gene expression of SOCS-1/SSI-1/JAB and SOCS-3/SSI-3/CIS-3, herein referred to as SOCS-1 and SOCS-3, are induced by IL-6 and LIF in various tissues. Both, SOCS-1 and SOCS-3 proteins bind to the JH1 domain of Jak-2 and thereby inhibit IL-6-, IL-11-, or LIF-induced tyrosine phosphorylation activity (by Jak-2) of gp130 and STAT-3liii. The transcription repressor BCL-6 binds to several sequence motifs scattered in the IL-6 locus and can repress IL-6 transcription both in 293T cells and in macrophages. BCL-6 negatively regulates proliferation of the monocytic/macrophage lineage by suppressing an autocrine IL-6/STAT3-mediated gene expression program. BCL-6(−/−) macrophages hyperproliferate due to an accelerated G(1)/S transition accompanied by increased cyclin D2 and c-myc and decreased expression of p27. Crucial to this enhanced proliferation is spontaneous interleukin 6 (IL-6) production and signal transducer and activator of transcription 3 (STAT3) activation in BCL-6(−/−) macrophagesliv.

TYROSINE KINASES Tyrosine-specific protein kinases (tyrosine kinases) represent a family of enzymes which catalyze the transfer of the terminal phosphate of adenosine triphosphate to tyrosine residues in protein substrates.

Tyrosine kinases consist of three general subclasses: (1) membrane receptor tyrosine kinases, including the insulin receptor and receptors for epidermal growth factor and platelet-derived growth factor; (2) cytosolic non-receptor protein tyrosine kinases which include members of the Src, Tec, JAK, Fes, Abl, FAK, Csk, and Syk families. (3) membrane-associated non-receptor tyrosine kinases which are associated with viral genes (oncogenes), capable of cell transformation and related closely to pp60v-src.14). JAK kinases; (abbreviation. for janus-activated kinase) is the name given to a family of non-receptor protein tyrosine kinases, comprising JAK1 (Janus kinase-1), JAK2 (Janus kinase-2), Tyk2 (non-receptor protein tyrosine kinase-2), which are widely expressed and JAK3 (Janus kinase-3) which is mainly found in cells of haematopoietic origin. Tyrosine-kinase receptors exist as single polypeptides in the plasma membrane. The extracellular portion of the protein, with the signal-molecule binding site, is connected by a single transmembrane a helix to the protein's cytoplasmic portion. This part of the protein is responsible for the receptor's tyrosine-kinase activity and also has a series of tyrosine amino acids. When signals molecules (such as a growth factor) attach to their binding sites, two polypeptides aggregate, forming a dimer. Using phosphate groups from ATP, the tyrosine-kinase region of each polypeptide phosphorylates the tyrosines on the other polypeptide. Thus, the dimer is both an enzyme and its own substrate. Now fully activated, the receptor protein can bind specific intracellular proteins, which attach to specific phosphorylated tyrosines and are themselves activated. Each can then initiate a signal-transduction pathway leading to a specific cellular response. Tyrosine-kinase receptors often activate several different signal-transduction pathways at once, helping regulate such complicated functions as cell reproduction (cell divisions). Inappropriate activation of these receptors can lead to uncontrolled cell growth—cancer. Tyrosine kinases are key elements in cellular signal transduction pathways. Small GTPases of the Ras protein superfamily stimulate the tyrosine phosphorylation and activation of the JAK family of intracellular kinases. This in turn activates the STAT family of transcription factors and results in the induction of Interleukin-6 and IL-6 receptor gene. STATS comprise a family of seven transcription factors that are activated by a variety of cytokines, hormones and growth factors. Engagement of cell surface Interleukin-6 receptors activates the Janus kinase (JAK) family of tyrosine kinases, which in turn phosphorylate the cytoplasmic part of gp130, thereby creating docking sites for STAT factors STAT1 and STAT3. Activated STATs dimerize upon activation by JAKs and translocate to the nucleus where they bind specific DNA response elements and regulate the expression of certain genes. Following gp130 dimerization, IL-6 activates multiple signaling pathways (Ras dependent MAP Kinase cascade, STAT1-STAT3 heterodimer pathway, and STAT3 homodimer pathway)

Protein tyrosine kinases (PTKs) play a key role in the regulation of cell proliferation, differentiation, metabolism, migration, and survival.

Extracellular interaction with a specific growth factor (ligand), initiates tyrosine kinase mediated signal transduction followed by receptor dimerization, transient stimulation of the intrinsic protein tyrosine kinase activity and phosphorylation. Binding sites are thereby created for intracellular signal transduction protein molecules and lead to the formation of complexes with a spectrum of cytoplasmic signaling molecules that facilitate the appropriate cellular response (e.g., cell proliferation, differentiation and metabolism, opening or closing of an ion channel in the plasma membrane, synthesis of enzymes or other proteins, usually by turning specific genes on or off). Many of the signaling protein components as well as the receptor itself consist of modular domains (small stretch of the polypeptide sequence which folds into a discrete domain) which determine protein-protein interactions necessary for the recruitment of particular proteins into signaling complexes. These are called modular domains include SH2, SH3, PTB, PH domains and others such as WW domains (small modules of 35-40 residues which bind proline rich motifs); PDZ domains.

Kinase enzymes catalyze the transfer of phosphoryl group. Phosphatase enzymes catalyze the removal of phosphoryl group. Phosphorylation takes place on one of three amino acid residues (serine, threonine or tyrosine) and utilises the □ phosphate of ATP. Enzymes that catalyse protein phosphorylation include serine/threonine kinases and the tyrosine kinases. Enzymes that catalyse dephosphorylation include Phosphoserine/threonine phosphatases and Phosphotyrosine phosphatases.

SERINE/THREONINE KINASES Serine/Threonine kinases include phosphorylase kinase (GPK), pyruvate dehydrogenase kinase, cAMP-dependent protein kinases (PKA), cGMP-dependent protein kinases (PKG), Protein kinase C (PKC), Ca2+/calmodulin-dependent protein kinases, G protein-coupled receptor kinases (GRKs), Mitogen-activated Protein kinases (MAP kinase), several oncogenes (including mil, raf and mos), haem-regulated protein kinase, plant-specific serine/threonine kinases, and Receptor serine/threonine kinases (receptors for transforming growth factor TGF-□ superfamily).

DIMERIC TRANSCRIPTION FACTORS Activator protein-1 (AP-1) is a collective term referring to dimeric transcription factors composed of Jun, Fos, or ATF (activating transcription factor) subunits that bind to the AP-1 binding site on the several proinflammatory genes including the IL-6 promoterlv. AP-1 activity plays an important role in

the inflammatory response by modulating gene expression of several inflammatory mediators including IL-6 transcription. Phosphorylation of c-Jun is a prerequisite of AP-1 dimerization and

activation (32). AP-1 activity is controlled by signaling through the JNK family of MAP kinases. It has been demonstrated that during reperfusion, oxidative stress leads to activation

and translocation of JNK to the nucleus, where phosphorylation of transcription factors, such as c-Jun occurs.

NUCLEAR FACTOR KAPPA B Nuclear factor κB (NF-κB) is a widely expressed, inducible transcription factor of particular importance to cells of the immune system. It was originally identified as an enhancer binding protein for the Ig K-light chain gene in B cells lvi. NF-κB regulates the expression of many genes involved in mammalian immune and inflammatory responses, including cytokines, cell adhesion molecules, complement factors, and a variety of immunoreceptors. The NF-κB transcription factor is a heterodimeric protein that comprises the p50 and p65 (Rel A) subunits. These subunits are proteins of the Rel family of transcriptional activators. Members of the Rel family share a conserved 300-amino acid Rel homology domain responsible for DNA binding, dimerization, and nuclear localization. While transcriptionally active homodimers of both p50 and p65 can form, the p50/65 heterodimer is preferentially formed in most cell typeslvii.

In the absence of stimulatory signals, the NF-κB heterodimer is retained in the cytoplasm by its physical association with an inhibitory phosphoprotein, IκB. Multiple forms of IκB have been identifiedlviii. Two of these forms, IκBα and IκBβ, have been shown to modulate the function of the NF-κB heterodimer, and these two IκBs are phosphorylated in response to different extracellular stimuli lix. Recent studies indicate that the catalytic subunit of protein kinase A (PKAC) is associated with the NF-κB/IκBα complexlx. In this p50/p65/IκBα/PKAC tetrameric configuration, IκBα renders PKAC inactive and masks the nuclear localization signal on NF-κB. Proinflammatory stimuli can activate a number of protein kinases, which have the capacity to modulate nuclear factor-κB (NF-κB) or activator protein-1 (AP-1) activity. A variety of extracellular stimulatory signals, such as cytokines, viruses, and oxidative stressorslxi activate kinases that phosphorylate IκB. The cytokine-activated IκB kinase termed IKK is the key regulatory kinase for IκBαlxii. IkappaB kinase (IKK) complex is composed of subunits, IKK-alpha, IKK-beta and IKK-gamma, which are serine/threonine protein kinases whose function is needed for NF-kappaB activation by pro-inflammatory stimulilxiii. Phosphorylation at serines 32 and 36 targets IκBα for ubiquitination and subsequent rapid proteolysis via a proteasome-mediated pathwaylxiv lxv lxvi lxvii, resulting in the release of NF-κB/PKAC. The now active PKAC subunit dissociates and phosphorylates the p65 subunit of NF-κB. Phosphorylated NF-κB then translocates to the cell nucleus, where it binds to target sequences in the chromatin and activates specific gene subsets, particularly those important to immune and inflammatory functionlxviii lxix lxx. PPAR alpha (Peroxisome proliferator-activated receptor alpha) negatively interferes with inflammatory gene expression by up-regulation of the cytoplasmic inhibitor molecule IkappaB alpha, thus establishing an autoregulatory loop. This induction takes place in the absence of peroxisome proliferator-response elements (PPRE), but requires the presence of NF-kappaB and Spl elements in the IkappaB alpha promoter sequence as well as DRIP250 cofactorslxxi.

IL-6 is encoded by a highly inducible promoter that is a target for tissue-specific and cytokine-inducible transcription factorslxxii lxxiii Interleukin-6 (IL-6) is expressed by angiotensin II (Ang II)-stimulated vascular smooth muscle cells (VSMCs). Ang II induces IL-6 transcription in a manner completely dependent on the nuclear factor-kappaB (NF-kappaB). One study analyzed the mechanism for Ang II-inducible IL-6 expression in quiescent rat VSMCs. Stimulation with the Ang II agonist Sar1 Ang II (100 nmol/L) induced transcriptional expression of IL-6 mRNA transcripts of 1.8 and 2.4 kb. In transient transfection assays of IL-6 promoter/luciferase reporter plasmids, Sar1 Ang II treatment induced IL-6 transcription by inducing cytoplasmic-to-nuclear translocation of the NF-kappaB subunits Rel A and NF-kappaB1 with parallel changes in DNA-binding activity in a biphasic manner, which produced an early peak at 15 minutes followed by a nadir 1 to 6 hours later and a later peak at 24 hours. The early phase of NF-kappaB translocation was dependent on weak simultaneous proteolysis of the IkappaBalpha and beta inhibitors, whereas later translocation was associated with enhanced processing of the p105 precursor into the mature 50-kDa NF-kappaB1 form. Pretreatment with a potent inhibitor of IkappaBalpha proteolysis, TPCK, completely blocked Sar1 Ang IIAng II-induced NF-kappaB activation and induction of endogenous IL-6 gene expression, which indicated the essential role of NF-kappaB in mediating IL-6 expression. The study authors concluded that Ang II is a pleiotropic regulator of the NF-kappaB transcription factor family and may be responsible for activating the expression of cytokine gene networks in VSMCslxxiv.

Peroxisome Proliferator-Activated Receptors (PPARs)

Peroxisome proliferator-activated receptors (PPARs) are ligand-activated transcription factors which form a subfamily of the nuclear receptor gene family. The PPAR subfamily consists of three isotypes, alpha (NR1C1), gamma (NR1C3), and beta/delta (NRC1C2) with a differential tissue distribution. PPARs are activated by ligands, such as naturally occurring fatty acids, which are activators of all three PPAR isotypes. In addition to fatty acids, several synthetic compounds, such as fibrates and thiazolidinediones, bind and activate PPARalpha and PPARgamma, respectively. PPARalpha is expressed primarily in tissues with a high level of fatty acid catabolism such as liver, brown fat, kidney, heart and skeletal muscle. PPARbeta is ubiquitously expressed, and PPARgamma has a restricted pattern of expression, mainly in white and brown adipose tissues, whereas other tissues such as skeletal muscle and heart contain limited amounts. Furthermore, PPARalpha and gamma isotypes are expressed in vascular cells including endothelial and smooth muscle cells and macrophages/foam cells. In order to be transcriptionally active, PPARs need to heterodimerize with the retinoid-X-receptor (RXR). Upon activation, PPAR-RXR heterodimers bind to DNA specific sequences called peroxisome proliferator-response elements (PPRE) and stimulate transcription of target genes. PPARs play a critical role in lipid and glucose homeostasis, but lately they have been implicated as regulators of inflammatory responses. The first evidence of the involvement of PPARs in the control of inflammation came from the PPARalpha null mice, which showed a prolonged inflammatory response. PPARalpha activation results in the repression of NF-kappaB signaling and inflammatory cytokine production in different cell-types. A role for PPARgamma in inflammation has also been reported in monocyte/macrophages, where ligands of this receptor inhibited the activation of macrophages and the production of inflammatory cytokines (TNFalpha, interleukin 6 and 1beta).lxxv. PPAR activators have effects on both metabolic risk factors and on vascular inflammation related to atherosclerosis. PPAR have profound effects on the metabolism of lipoproteins and fatty acids. PPAR alpha binds hypolipidemic fibrates, whereas PPAR gamma has a high affinity for antidiabetic glitazones. Both PPAR alpha and gamma are activated by fatty acids and their derivatives. Activation of PPAR alpha increases the catabolism of fatty acids at several levels. In the liver, it increases uptake of fatty acids and activates their beta-oxidation. The effects that PPAR alpha exerts on triglyceride-rich lipoproteins is due to their stimulation of lipoprotein lipase and repression of apolipoprotein CIII expression, while the effects on high-density lipoproteins depend upon the regulation of apolipoproteins AI and AII. PPAR gamma has profound effects on the differentiation and function of adipose tissue, where it is highly expressed. PPAR are also expressed in atherosclerotic lesions and are present in vascular endothelial cells, smooth muscle cells, monocytes, and monocyte-derived macrophages. Via negative regulation of nuclear factor-kappa B and activator protein-1 signalling pathways, PPAR alpha inhibits expression of inflammatory genes, such as interleukin-6, cyclooxygenase-2, and endothelin-1. Furthermore, PPAR alpha inhibits expression of monocyte-recruiting proteins such as vascular cell adhesion molecule (VCAM)-1 and induces apoptosis in monocyte-derived macrophages. PPAR gamma activation in macrophages and foam cells inhibits the expression of activated genes such as inducible nitric oxide synthase, matrix metalloproteinase-9 and scavenger receptor A. PPAR gamma may also affect the recruitment of monocytes in atherosclerotic lesions as it is involved in the expression of VCAM-1 and intracellular adhesion molecule-1 in vascular endothelial cellslxxvi.

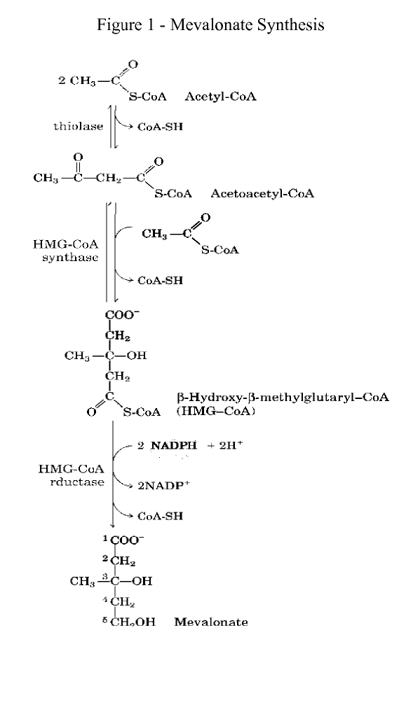

CHOLESTEROL METABOLISM Normal healthy adults synthesize cholesterol at a rate of approximately 1 g/day and consume approximately 0.3 g/day. A relatively constant level of cholesterol in the body (150-200 mg/dL) is maintained primarily by controlling the level of de novo synthesis. The level of cholesterol synthesis is regulated in part by the dietary intake of cholesterol. Cholesterol from both diet and synthesis is utilized in the formation of membranes and in the synthesis of the steroid hormones and bile acids. The greatest proportion of cholesterol is used in bile acid synthesislxxvii. Cholesterol synthesis occurs in the cytoplasm and microsomes with initial synthesis of mevalonate from the two-carbon acetate group of acetyl-CoA. See FIG. 1 (Mevalonate Synthesis).

1. Synthesis begins when acetyl-CoA is derived from an oxidation reaction in the mitochondria and is transported to the cytoplasm

2. Two moles of acetyl-CoA are condensed, forming acetoacetyl-CoA. Acetoacetyl-CoA and a third mole of acetyl-CoA are converted to 3-hydroxy-3-methylglutaryl-CoA (HMG-CoA) by the action of HMG-CoA synthase.

3. HMG-CoA is converted to mevalonate, in a rate limiting step catalyzed by the enzyme HMG-CoA reductase, (HMGR)

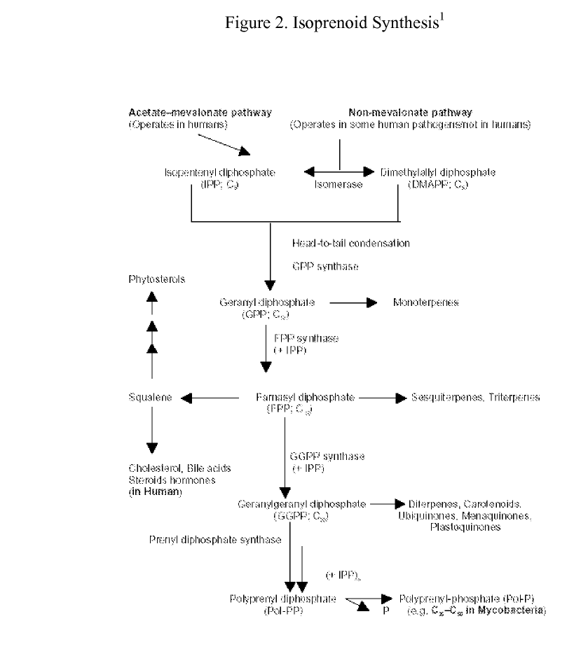

In human beings, cholesterol and isoprenoids are then synthesized via the mevalonate pathway. See FIG. 2 (Cholesterol and Isoprenoid Synthesis).

1. Mevalonate is activated by three successive phosphorylations, yielding 5-pyrophosphomevalonate

2. After phosphorylation, an ATP-dependent decarboxylation yields isopentenyl pyrophosphate, (IPP), an activated isoprenoid molecule. Isopentenyl pyrophosphate is in equilibrium with its isomer, dimethylallyl pyrophosphate, DMAPP.

3. One molecule of IPP condenses with one molecule of DMAPP to generate geranyl pyrophosphate, (GPP). This step is catalyzed by GPP synthase.

4. GPP further condenses with another IPP molecule to yield farnesyl pyrophosphate, (FPP). This step is catalyzed by FPP synthase.

5. FPP condenses with another IPP molecule to yield geranylgeranyl pyrophosphate (GGPP). This step is catalyzed by GGPP synthase

6. The head-to-tail condensation of two molecules of FPP yielding Squalene, is catalyzed by squalene synthase.

7. Squalene undergoes a two-step cyclization to yield lanosterol.

8. Lanosterol is converted to cholesterol, through a series of 19 additional reactions

There is a complex regulatory system to co-ordinate the biosynthesis of cholesterol with the availability of dietary cholesterol. The cellular supply of cholesterol is maintained at a steady level by the following mechanisms:

1. Regulation of HMGR activity and levels

2. Regulation of excess intracellular free cholesterol through the activity of acyl-CoA:cholesterol acyltransferase, (ACAT)

3. Regulation of plasma cholesterol levels via LDL receptor-mediated uptake and HDL-mediated reverse transport.

ACTIVATION OF INTERLEUKIN-6 INFLAMMATION BY ISOPRENOIDS Cytokine receptors act through a complex signaling network involving GTPase proteins such as Ras, Rho, Rac, and Rab (particularly Rho), Janus kinases (JAKs) and the signal transducers and activators of transcription (STATs) to regulate diverse biological processes controlling immune function, growth, development and homeostasislxxviii.

Isoprenoids are necessary for posttranslational lipid modification (prenylation) and, hence, the function of Ras and other small guanosine triphosphatases (GTPases)lxxix.

GTPase proteins such as Ras, Rho, Rac, and Rab (particularly Rho) are intracellular signaling proteins that, when activated, are involved in receptor-coupled transduction of signals from extracellular stimuli to cytoplasm and the nucleus. Small GTPase proteins constitute a Ras superfamily, which is comprised of at least five major branches. Members of the Ras branch include the Ras, Rap, Ral and R-Ras family proteinslxxx lxxxi. The Ras family regulates gene expression. The Rho branch constitutes a second major branch, with RhoA, Rac1 and Cdc42 the most studied members. The Rho family regulates cytoskeletal reorganization and gene expression. The Rab branch is the largest, and, together with members of the Arf/Sar branch, serve as regulators of intracellular vesicular transport. Ran is the sole member of its branch and is a crucial regulator of nucleo-cytoplasmic transport of proteins and RNA. The Ras superfamily proteins alternate between an inactivated GDP-bound form and activated GTP-bound form, allowing them to act as molecular switches for growth and differentiation signals. Prenylation is a process involving the binding of hydrophobic isoprenoid groups consisting of farnesyl or geranylgeranyl residues to the C-terminal region of Ras protein superfamily. Farnesyl pyrophosphate (FPP) and geranylgeranyl pyrophosphate (GPP) are metabolic products of mevalonate that are able to supply prenyl groups. The prenylation is conducted by prenyl transferases. The hydrophobic prenyl groups are necessary to anchor the Ras superfamily proteins to intracellular membranes so that they can be translocated to the plasma membranelxxxii. The final cell-membrane fixation is necessary for Ras proteins to participate in their specific interactionslxxxiii lxxxiv. The activity of the small GTPase, Rac1, plays a role in various cellular processes including cytoskeletal rearrangement, gene transcription, and malignant transformation. Small GTPases of the Ras protein superfamily stimulate the tyrosine phosphorylation and activation of the JAK family of intracellular kinases. This in turn activates the STAT family of transcription factors and results in the induction of Interleukin-6 and IL-6 receptor gene. Persistent Rac1 activity leads to the autocrine production and signal transduction of Interleukin-636. IL-6 itself may produce a delayed phosphorylation and activation of STAT3, and the JAK/STAT3 pathway is an indirect target of Ras and Rho GTPaseslxxxv. Blocking the IL-6 signaling pathway inhibits Rac1-mediated STAT3-dependent gene expression. In one studylxxxvi, constitutively active Rac1 (Rac V12) is shown to stimulate the activation of STAT3. The activity of Rac1 leads to STAT3 translocation to the nucleus coincident with STAT3-dependent gene expressionlxxxvii. The study indicated that Rac1 induces STAT3 activation through an indirect mechanism that involves the autocrine production and action of IL-6, which is a known mediator of STAT3 response. Rac1 expression results in the induction of the IL-6 and IL-6 receptor genes and neutralizing antibodies directed against the IL-6 receptor block Rac1-induced STAT3 activation. Inhibition of nuclear factor-kappaB activation or disruption of IL-6-mediated signaling through the expression of IkappaBalpha S32AS36A and suppressor of cytokine signaling 3, respectively, blocks Rac1-induced STAT3 activation. The study also investigated whether the other Rho family members mediate STAT3 activation in an IL-6-dependent pathway. The expression of constitutively active RhoG, Cdc42, and RhoA caused the translocation from the cytoplasm to the nucleus of cotransfected STAT3-GFP. This GTPase-induced STAT3 translocation was blocked to varying degrees by neutralizing IL-6 receptor antibodies, supporting a role for autocrine IL-6 in Rho family-induced STAT3 activation These findings elucidate a mechanism dependent on the induction of an autocrine IL-6 activation loop through which Rac1 and the Rho family mediate STAT3 activation establishing a link between GTPase activity and Janus kinase/STAT signaling. Interestingly, STAT3 is persistently activated in many human cancers and transformed cell lines. In cell culture, active STAT3 is either required for transformation, enhances transformation, or blocks apoptosis.

In one studylxxxviii, leukemic cells from 50 patients with acute myeloid leukemia (AML) were analyzed for the presence of activating point mutations of the N-RAS gene using polymerase chain reaction (PCR) and differential oligonucleotide hybridization. This assay allows semiquantitative determination of the relative abundance of cells carrying N-RAS mutations. Clonal activation of N-RAS, noted in the large majority of leukemic cells of the six of these patients, was correlated significantly (p=0.0003) with the ability of these cells to express interleukin 6 (IL-6), previously shown to be expressed at high levels in approximately 30% of primary AML cells. Another study investigated the effect of a nonpeptidomimetic farnesyl transferase inhibitor R115777 in the Ras/MAPK and JAK/STAT pathways, which are implicated in survival and/or proliferation in Multiple Myeloma (MM). The phosphorylation of both STAT3 and ERK1/2 induced by IL-6 was totally blocked at 15 microM of R115777 and partially blocked when R115777 was used at 10 and 5 microM. R115777 induced (1) a significant and dose-dependent growth inhibition of the three myeloma cell lines tested; and (2) a significant and time-dependent apoptosis. R115777 also induced apoptosis in the bone marrow mononuclear cell population of four MM patients, being almost restricted to the malignant plasma cellslxxxix.

In summary, isoprenoids farnesyl pyrophosphate (FPP) and geranylgeranyl pyrophosphate (GPP) are necessary for posttranslational lipid modification (prenylation) and, hence, the function of Ras and other small GTPase proteins such as Ras, Rho, Rac, and Rab52. Persistently active Rho family and Rac1 results in the activation of JAKs and subsequent tyrosine phosphorylation and activation of STAT3xc. Tyrosine phosphorylated STAT3 forms dimers that translocate to the nucleus to bind DNA target sites in responsive genes59. IL-6 and IL-6 receptor gene induction occurs as a result of activated STAT proteins and IL-6 mediates the long-term activation of STAT3 through an autocrine loop.

ACTIVATION OF INTERLEUKIN-6 INFLAMMATION BY ACTIVATED MONOCYTES IN THE INFLAMMATORY RESPONSE TO INFECTION AND TRAUMA HMG-CoA reductase generates mevalonate, the precursor of a complex series of isoprenoids molecules that are necessary for posttranslational lipid modification (prenylation) and, hence, the function of intracellular signaling proteins that, when activated, are involved in expression of Interleukin 6 mediated inflammation. Tissue injury, subsequent to a physical, chemical or biological insult, results in an inflammatory response associated with invasion of the area by immune cells, which include monocytes, T helper cells, lymphocytes, neutrophils, eosinophils, and other cells such as fibroblasts and endothelial cells. Isoprenoids are required for NADPH oxidase activity (reduced nicotinamide adenine dinucleotide phosphate) in granulocytes via low-molecular-weight (LMW) GTP-binding protein isoprenylation. Isoprenoid generation through the mevalonate pathway is a requirement for IL-8 and IL-6 induction by activated monocytic cells in vitro. One study evaluated the effects of isoprenoid depletion on the expression of proinflammatory genes in human monocytic THP-1 cells. The researchers selected conditions under which pretreatment for 24 h with isoprenoid synthesis inhibitors (HMG-CoA reductase inhibitor lovastatin or compactin at 10 microM) did not compromise cell viability but markedly suppressed hydrogen peroxide (H2O2) generation. Under these conditions interleukin-8 (IL-8) production was attenuated (by 50-90%) in response to lipopolysaccharide, granulocyte-macrophage colony-stimulating factor, and phorbol myristate acetate. Coincubation of reductase inhibitor-treated cells with mevalonate prevented the attenuation of IL-8 production by reductase inhibitors. The effects of isoprenoid depletion on cytokine production were selective: IL-1 beta generation was not inhibited but the production of IL-6 and IL-8 was concomitantly suppressed. IL-8 induction was suppressed at least in part through attenuation of the increase in mRNA in stimulated cells. The study authors concluded that isoprenylation inhibitors have the potential to alter monocyte proinflammatory functionxci. In another study, fluvastatin decreased (and mevalonate rescued) signaling molecules within membrane rafts in monocytes in parallel with effects on tyrosine phosphorylation events. In addition, Fcgamma receptor mediated immune complex trafficking, activation of MAP kinases (ERK and p38), and downstream inflammatory mediator release (MMP-1 and IL-6) were blocked by fluvastatin. The study authors concluded that HMG-CoA reductase inhibition alters immune receptor signaling in monocytes by disrupting membrane rafts essential for the initiation of signal transductionxcii. Another study explored the role of mevalonate inhibitors in the activation of nuclear factor kappa B (NF kappa B) and the induction of inducible nitric oxide synthase (iNOS) and cytokines (TNF-alpha, IL-1beta, and IL-6) in rat primary astrocytes, microglia, and macrophages. Lovastatin and sodium phenylacetate (NaPA) were found to inhibit Lipopolysaccharide (LPS) and cytokine-mediated production of NO and expression of iNOS in rat primary astrocytes; this inhibition was not due to depletion of end products of mevalonate pathway (e.g., cholesterol and ubiquinone). The authors stated that reversal of the inhibitory effect of lovastatin on Lipopolysaccharide (LPS)-induced iNOS expression by mevalonate and farnesyl pyrophosphate and reversal of the inhibitory effect of NaPA on LPS-induced iNOS expression by farnesyl pyrophosphate suggests a role of farnesylation in the LPS-mediated induction of iNOS. The inhibition of LPS-mediated induction of iNOS by FPT inhibitor II, an inhibitor of Ras farnesyl protein transferase, suggests that farnesylation of p21(ras) or other proteins regulates the induction of iNOS. Inhibition of LPS-mediated activation of NF kappa B by lovastatin, NaPA, and FPT inhibitor II in astrocytes indicates that the observed inhibition of iNOS expression is mediated via inhibition of NF kappa B activation. In addition to iNOS, lovastatin and NaPA also inhibited LPS-induced expression of TNF-alpha, IL-1beta, and IL-6 in rat primary astrocytes, microglia, and macrophages. The authors concluded that their study delineates a novel role of the mevalonate pathway in controlling the expression of iNOS and different cytokines in rat astrocytes, microglia, and macrophages that may be important in developing therapeutics against cytokine- and NO-mediated neurodegenerative diseasesxciii.

Bacterial infection as typified by periodontal disease is associated with inflammation and the inflammatory response, with generation of isoprenoids by activated monocytes. Bacteria also directly synthesize isoprenoid molecules by a mevalonate—independent (non-MVA) pathway (see FIG. 1). The synthesis of IPP and DMAPP via the non-MVA pathway starts with the formation of 1-deoxy-Dxylulose-5-phosphate (DOXP) by two glycolytic intermediates, pyruvate and glyceraldehyde-3-phosphatexciv. These isoprenoids may be involved in the cell-wall biosynthesis and may also play a role in direct activation of biologically active mediatorsxcv. Periodontal disease is characterized by adherence and colonization of the tooth enamel and root surface by saccharolytic, aerobic Streptococcus species. and other bacteria. This sets the stage for Fusobacterium nucleatum to coaggregate with these early colonizers and to permit late colonizers, including dental pathogens, to eventually form a biofilm. These complex interactions result in the release of factors that lead to tooth decayxcvi. In a landmark study in Finland, Matilla et al examined the role of chronic bacterial infections as risk factors for coronary heart disease. The association between poor dental health and acute myocardial infarction was investigated in two separate case-control studies of a total of 100 patients with acute myocardial infarction and 102 controls selected from the community at random. Dental health was graded by using two indexes, one of which was assessed blind. Based on these indexes dental health was significantly worse in patients with acute myocardial infarction than in controls. The association remained valid after adjustment for age, social class, smoking, serum lipid concentrations, and the presence of diabetesxcvii. More recently, these results were confirmed in studies in the United States, Canada, Great Britain, Sweden, and Germany. In another study, Morrison et al found that people with periodontal disease had a factor of 2 higher risk of dying from cardiovascular disease. By comparison smokers only had a 60% increased riskxcviii. Meyer et al showed that c-reactive proteins and pro-inflammatory cytokines are released during periodontal flare-ups and capable of eliciting effects associated with atherosclerosis and coronary heart diseasexcix. The presence of oral infections is also associated with cerebrovascular disease, strokec, preterm birthsci, osteoporosiscii and type 2 diabetes. One study evaluated 113 Pima Indians with both diabetes and periodontal disease. The study found that when their periodontal infections were treated, the management of their diabetes markedly improvedciii.

INHIBITION OF CHOLESTEROL PATHWAY BY STATINS The main effect of statins is the decrease of serum level of low-density lipoprotein (LDL) cholesterol, due to the inhibition of intracellular cholesterol biosynthesis. A minor effect is the decrease of serum triglycerides. Statins inhibit HMG-CoA reductase and decrease the production of mevalonate, geranyl pyrophosphate, and farnesyl pyrophosphate, and subsequent products on the way to construction of the cholesterol molecule. Thus, statins could inhibit inflammation, by inhibition of the cholesterol pathway and intracellularly interfering with Ras superfamily protein functionciv. Ikeda et al.cv recently showed that statins decrease matrix metalloproteinase-1 expression through inhibition of Rho. Statin therapy has been demonstrated to provide significant reductions in non-high-density lipoprotein cholesterol, and to decrease cardiovascular morbidity and mortality.

INHIBITION OF CHOLESTEROL PATHWAY BY BISPHOSPHONATES Recent findings suggest that alendronate and other N-containing bisphosphonates inhibit the isoprenoid biosynthesis pathway and interfere with protein prenylation, as a result of reduced geranylgeranyl diphosphate levels. One studycvi utilizing High-performance liquid chromatography (HPLC) analysis of products from a liver cytosolic extract, identified farnesyl disphosphate (FPP) synthase as the mevalonate pathway enzyme inhibited by bisphosphonates. Recombinant human farnesyl diphosphate synthase was inhibited by alendronate with an IC(50) of 460 nM (following 15 min preincubation). Alendronate did not inhibit isopentenyl diphosphate isomerase or GGPP synthase. Recombinant farnesyl diphosphate synthase was also inhibited by pamidronate (IC(50)=500 nM) and risedronate (IC(50)=3.9 nM), negligibly by etidronate (IC50=80 microM), and not at all by the non-nitrogen-containing bisphosphonate clodronate. In another study, a wide range of bisphosphonates, were found to have a significant correlation between potency for inhibition of recombinant human FPP synthase in vitro and anti-resorptive potency in vivo, suggesting that this enzyme is the major pharmacologic target of these drugs. The most potent anti-resorptive bisphosphonates such as zoledronic acid and risedronate are very potent inhibitors of FPP synthase, with IC50 values as low as 3 nM and 10 nM respectively. Inhibition of FPP synthase prevents the formation of FPP and its derivative GGPP. These isoprenoid lipids are necessary for the post-translational lipid modification (prenylation) of small GTPase proteins such as Ras, Rho, Rac, and Rab. The effects of nitrogen-containing bisphosphonates on osteoclasts can be overcome by addition of components of the mevalonate pathway, which bypass the inhibition of FPP synthase and restore protein prenylation. In particular, geranylgeraniol (a cell-permeable form of GGPP) prevents inhibition of resorption by nitrogen-containing bisphosphonates in vitro.cvii. One study aimed to evaluate cholesterol and lipoprotein serum levels in patients with Paget's bone disease treated with intravenous pamidronate. The study included 20 consecutive patients (mean age, 67.6+/−11.0 years) with Paget's bone disease for at least 1 year, who needed intravenous amino bisphosphonate treatment; 12 patients with inactive Paget's bone disease served as controls. The patients with active Paget's bone disease underwent three cycles (every 3 months) of treatment with 60 mg of intravenous pamidronate. Controls were given a saline infusion following the same administration schedule. In all subjects total alkaline phosphatase (total ALP), bone alkaline phosphatase (bone ALP), total cholesterol (TC), tryglycerides (TG), and high- and low-density lipoprotein cholesterol (HDL-C and LDL-C, respectively) were measured before infusions (pamidronate or saline) at baseline and at 3-month intervals up to 9 months. In the control group no significant changes were observed through the study period for any of the biochemical parameters. In the pamidronate-treated patients, both bone ALP and total ALP significantly fell at the end of the study. In patients with active treatment, at the end of the study period HDL-C significantly (P<0.05) increased by 10.3%, whereas LDL-C significantly (P<0.05) decreased by 5.5%. In these patients TC showed a negative trend without reaching statistical significance, whereas the HDL-C/LDL-C ratio rose 16.2% above the basal value and TC/HDL-C decreased by 12.5%. The researchers concluded that, pamidronate given intravenously seems to be able to induce a prolonged shifting in circulating cholesterol from the LDL-C to the HDL-C that is associated with a weak decrease in total cholesterol, thus producing a possible improvement in the atherosclerotic risk indexcviii.

FOOD PRODUCTS—FUNGI AND POLYPHENOLIC COMPOUNDS Statins identical to the cholesterol lowering pharmaceutical lovastatin and its derivatives of simvastatin, pravastatin and mevastatin can be produced by a variety of filamentous fungi, including Monascus, Aspergillus, Penicillium, Pleurotus, Pythium, Hypomyces, Paelicilomyces, Eupenicillium, and Doratomyces cix. As a food product, rice fermented with a red Monascus fungus (red rice) has been known to contain low amounts of statins and used for hundreds of years in China. Red rice is used in wine making, as a food-coloring agent and as a drug in traditional Chinese medicine.

Several hundred molecules having a polyphenol (polyhydroxyphenol) structure (i.e. several hydroxyl groups on aromatic rings) have been identified in edible plants. These molecules are secondary metabolites of plants and are generally involved in defense against ultraviolet radiation or aggression by pathogens. Polyphenols are widespread constituents of fruits, vegetables, cereals, dry legumes, chocolate, and beverages, such as tea, coffee, or wine.

These compounds may be classified into different groups as a function of the number of phenol rings that they contain and of the structural elements that bind these rings to one another. Classes of polyphenols include the phenolic acids, flavonoids, stilbenes, and lignans. There are two classes of phenolic acids: derivatives of benzoic acid and derivatives of cinnamic acid.

Hydroxybenzoic acids are components of complex structures such as hydrolyzable tannins (gallotannins in mangoes and ellagitannins in red fruit such as strawberries, raspberries, and blackberries). Hydroxycinnamic acids are more common than are the hydroxybenzoic acids and consist chiefly of p-coumaric, caffeic, ferulic, and sinapic acids. Caffeic and quinic acid combine to form chlorogenic acid, which is found in many types of fruit and in high concentrations in coffee.

Flavonoids, are the largest single class as far as total numbers of known compounds. About two-thirds of the polyphenols we obtain in our diets are flavonoids. Flavonoids share a common structure consisting of 2 aromatic rings that are bound together by 3 carbon atoms that form an oxygenated heterocycle, and may be divided into 6 major subclasses: Anthocyanidins (e.g., cyanidin, pelargonidin); Flavanols (e.g., epicatechin, gallocatechin); Flavones (e.g., apigenin, luteolin); Flavonols (e.g., kaempferol, myricetin, quercetin); Flavanones (e.g., hesperidin, naringenin); Isoflavones (e.g., genistein, daidzein, biochanin) and Proanthocyanidinscx

Proanthocyanidins (condensed tannins) are a class of polyphenolic compounds found in several plant species. They include procyanidins, which are chains of catechin, epicatechin, and their gallic acid esters and the prodelphinidins, which consist of gallocatechin, epigallocatechin, and their gallic acid esters as the monomeric units.

Isoflavones are flavonoids with structural similarities to estrogens. Although they are not steroids, they have hydroxyl groups in positions 7 and 4 in a configuration analogous to that of the hydroxyls in the estradiol molecule. This confers pseudohormonal properties on them, including the ability to bind to estrogen receptors, and they are consequently classified as phytoestrogens.

Cocoa polyphenols comprise polyphenolic products including proanthocyanidins, particularly procyanidins, extracted from cocoa beans and derivatives thereof including fresh beans, defatted solids, comminuted trash beans, cocoa powder, low-fat cocoa powder, cocoa shells, cocoa waste. Polyphenols may be found in nuts, nut skin extracts, tea and tea derivatives, (e.g., Camelliasinensis, C. assamica), coffee beans (Coffeaarabica, C. aniphora, C. robusta, C. liberica) and derivatives thereof and cocoa beans (Theobroma cacao) and cocoa derivatives, grape juice and red wine

Phytoestrogenic isoflavones including genistein, daidzein, glycitein, biochanin A, formononetin, and their respective naturally occurring glycosides and glycoside conjugates are found in plants such as legumes, clover, and the root of the kudzu vine (pueraria root). Common legume sources of these isoflavone compounds include soy beans, chick peas, ground nuts, lentils and various other types of beans and peas. Clover sources of these isoflavone compounds include red clover and subterranean clover.

Genistein, (also known as 4′,5,7-trihydroxyisoflavone) is a common precursor in the biosynthesis of antimicrobial phytoalexins and phytoanticipins in legumes. Genistein is synthesized in plants from the flavanone naringenin. Genistein is a phytoestrogen with a wide variety of pharmacological effects in animal cells, including tyrosine kinase inhibition. Genistein has been shown to inhibit specifically in vitro the epidermal growth factor (EGF)-receptor tyrosine protein kinase activitycxi.

Soy is the richest dietary source of isoflavones. Typical soyfoods like tofu might provide 1-4 mg/g or about 40-100 mg of isoflavones per ounce. Soymilk provides about 100-150 mg of isoflavones per 8-ounce glass. The isoflavones function as phytoestrogens in the body, where they possess weak estrogen-like effects. The two primary isoflavones found in soy are daidzein and genistein. The chemical structure of isoflavones is similar enough to that of estrogen so that they can bind to the estrogen receptor on cells, yet different enough so that they only perform very weak estrogen effects. For the different soy-based protein powders on the market, the isoflavone content can vary significantly, from almost zero for those products extracted using alcohol, to certified levels of 2-5 mg per gram of protein. In many Asian countries, where the incidence of heart disease, cancer and menopausal symptoms is low, the daily isoflavone intake is estimated at 25-50 mg per day-in contrast, the average Western intake is less than 5 mg per day.

Soy beans are a particularly preferred source of the isoflavone compounds (except biochanin A and its glycosides which are not present in soy). Isoflavone compounds may be obtained from the plant sources in which they naturally occur or may be synthetically prepared.

Soy-based food products may be classified into two general categories. The first category consists of products manufactured from whole soybeans such as tofu, soynuts, soy milk, soy cheese, and soy yoghurt and products whose protein compositions are derived solely from soy protein products such soy flour, ST flour, ISP, and SPC. The second category of soy-based foods eligible for the claim consists of products manufactured in part using soybean-derived protein ingredients such as soy flour, ST flour, ISP, and SPC.

Phytosterols are sterol compounds produced by plants which are structurally very similar to cholesterol except that they contain some substitutions at the C.sub.24 position on the sterol side chain. Phytosterols include plant sterols, esters of plant sterols, plant stanols or stanol esters and stanols and stanol esters derivable from plant sterols. Examples include alpha sitosterol, beta sitosterol, stigmasterol, ergosterol, campesterol, alpha sitostanol, beta sitostanol, campestanol, oryzanol and brassiciasterol, their fatty acid esters, and the like. At least 44 phytosterols have been identified and it will be apparent to one of ordinary skill that many of these will be appropriate for the present invention. Important sources of phytosterols are rice bran, corn bran, corn germ, wheat germ oil, corn oil, safflower oil, oat oil, olive oil, cotton seed oil, soybean oil, e.g., soybean oil distillates, peanut oil, black tea, orange juice, valencia, green tea, Colocsia, kale, broccoli, sesame seeds, shea oils, grapeseed oil, rapeseed oil, linseed oil, canola oil, tall oil from wood pulp and other resinous oil from wood pulp. Phytosterols inhibit intestinal cholesterol absorption, thereby lowering blood total and low-density lipoprotein (LDL) cholesterol concentrations.

Food products according to the invention are preferably foods including fruits, nuts, vegetables and grains, dry legumes, chocolate, and beverages, such as tea, coffee, or wine, which contain polyphenolic compounds. These include phenolic acids, flavonoids, stilbenes, lignans, gallotannins, ellagitannins, hydroxybenzoic acids and other derivatives of benzoic acid, p-coumaric, caffeic, ferulic, sinapic, chlorogenic acids, hydroxycinnamic acids and other derivatives of cinnamic acid; flavonoids, anthocyanidins including cyanidin, pelargonidin; flavanols including epicatechin, gallocatechin; flavones including apigenin, luteolin; flavonols including kaempferol, myricetin, quercetin; flavanones including hesperidin, naringenin; isoflavones including genistein, daidzein, biochanin, proanthocyanidins (condensed tannins) including procyanidins, catechin, epicatechin, and their gallic acid esters, prodelphinidins including gallocatechin, epigallocatechin, and their gallic acid esters.

These also include food products in which soy protein materials are used as functional ingredients. They include, but are not limited to meats such as ground meats, emulsified meats, fermented meats and marinated meats, beverages such as nutritional beverages, sports beverages, protein fortified beverages, juices, milk, milk alternatives, and weight loss beverages, cheeses and cheese like products, such as tofu, frozen desserts such as ice cream, ice milk, low fat frozen desserts, and non-dairy frozen desserts, yoghurts, soups, sauces, such as soy sauce, puddings, breakfast cereals, pasta products, bakery products, such as bread and cake, salad dressings, and dips and spreads such as mayonnaise, chip dips, low fat spreads, sandwich spreads, dietetic products e.g. slimming products or meal replacers etc.

Fatty acids consist of chains of carbon atoms linked together by chemical bonds. Fatty acids come in different lengths: short chain fatty acids have fewer than 6 carbons, while long chain fatty acids have 12 or more carbons. On one terminal of the carbon chain is a methyl group and on the other terminal is a carboxyl group. The chemical bonds between the carbon atoms determine whether a fatty acid is saturated or unsaturated. Saturated fatty acids contain single bonds only. Examples of foods high in saturated fats include lard, butter, whole milk, cream, eggs, red meat, chocolate, and solid shortenings. An excess intake of saturated fat can raise blood cholesterol and increase the risk of developing coronary heart disease. Monounsaturated fatty acids contain one double bond. Examples of foods high in monounsaturated fat include avocados, nuts, and olive, peanut, and canola oils. Polyunsaturated fatty acids contain more than one double bond. Examples of foods high in polyunsaturated fats include vegetable oils, corn, sunflower, and soy. Essential fatty acids are polyunsaturated fatty acids that the human body needs for metabolic functioning but cannot produce, and therefore has to be acquired from food. Omega-3 fatty acids are a class of essential polyunsaturated fatty acids with the double bond in the third carbon position from the methyl terminal (hence the use of “3” in their description). Foods high in omega-3 fatty acids include cold-water fatty fish such as salmon, herring, mackerel, anchovies and sardines, and vegetable sources such as the oil from the seeds of chia, perilla, flax, purslane, hemp, and canola. Other foods that contain omega-3 fatty acids include whole grains, beans, green leafy vegetables such as spinach and seafood such as shrimp, clams, light chunk tuna, catfish and cod. Omega-6 fatty acids are a class of essential polyunsaturated fatty acids with the initial double bond in the sixth carbon position from the methyl group. Examples of foods rich in omega-6 fatty acids include corn, safflower, sunflower, soybean, and cottonseed oil.

Omega-3 and omega-6 fatty acids are also referred to as n-3 and n-6 fatty acids, respectively.

There are several different types of omega-6 fatty acids. Most omega-6 fatty acids are consumed in the diet from vegetable oils as linoleic acid (LA). Linoleic acid is converted to gamma-linolenic acid (GLA) in the body and then further broken down to arachidonic acid (AA). AA can also be consumed directly from meat, and GLA can be ingested from several plant-based oils including evening primrose oil (EPO), borage oil, and black currant seed oil. Excess amounts of LA and AA promote inflammation. There are three major types of omega 3 fatty acids that are ingested in foods: alpha-linolenic acid (ALA), eicosapentaenoic acid (EPA), and docosahexaenoic acid (DHA). Once eaten, the body converts ALA to EPA and DHA, the two types of omega-3 fatty acids more readily used by the body. The conversion of Omega-3 and Omega-6 fatty acids uses the same enzyme pools. Through a series of desaturations and elongations, LA is converted to AA, while ALA is converted to EPA and further elongated to DHA. AA and EPA reside in the membrane phospholipid bilayer of cells and are precursors to various eicosanoids. AA is a precursor to a group of eicosanoids that are proinflammatory and prothrombic, while EPA is a precursor to less metabolically active eicosanoids. AA is a precursor to series 2 prostaglandins and thromboxanes and series 4 leukotrienes. The series 2 and 4 eicosanoids metabolized from AA promote inflammation, and can act as vasoconstrictors, stimulate platelet aggregation and are potent chemotoxic agents. EPA is a precursor to series 3 prostaglandins and thromboxanes and series 5 leukotrienes. They are less potent than the series 2 and 4 counterparts and act as vasodilators and anti-aggregators.

Phospholipase A2 is activated by inflammatory stimuli and cleaves the fatty acid from the phospholipid bilayer. The fatty acids are then converted to eicosanoids via the cyclooxygenase (COX) or lipoxygenase (LOX) pathways. Omega-6 and Omega-3 fatty acids compete for the COX or LOX enzymes to form eicosanoids. Therefore, adequate EPA in phospholipid pools allows for a decrease in the amount of eicosanoids produced from AA possibly by reducing the amount of enzyme available for conversion of AA to pro-inflammatory prostaglandins and leukotrienescxii cxiii

Omega-3 fatty acids reduce inflammation and help prevent age related disorders including heart disease and arthritis. The omega-6 to omega-3 ratio of polyunsaturated fatty acids in the food is important, with an optimal ratio 4 to 1 in diet. Traditional western diets present absolute or relative deficiency of omega-3 polyunsaturated fatty acids, and a ratio 15-20 to 1.

Fatty fish are the major source of EPA and DHA in the diet, while vegetable oils, especially soybean and canola oils, are the primary sources of ALA. Flaxseed oil contains approximately 57-percent ALA, but it is not commonly used in food preparation. Nuts, seeds, vegetables, and some fruit, as well as egg yolk, poultry, and meat contribute small amounts of omega-3 fatty acids to the diet. Studies show that the consumption of increased amounts of fish, fish oil, flaxseed oil, or canola oil will result in the incorporation of the longer-chain omega-3 fatty acids EPA and DHA into the plasma and cell membranes of platelets, erythrocytes, neutrophils, monocytes, and liver cellscxiv cxv. This leads to a change in the ratio of omega-6:omega-3 fatty acids in the membranes, a change in the function of the membranes and a decrease in the production of IL-1, IL-6 and TNF-Alpha cxvi cxvii cxviii

ATHEROSCLEROSIS AND INTERLEUKIN 6 Macrophage uptake of oxidized low-density lipoprotein (Ox-LDL) is a hallmark of the early atherosclerotic lesion, and may be mediated by Interleukin-6. Incubation of IL-6 with MPM or IL-6 administration in mice increased macrophage Ox-LDL degradation and CD36 mRNA expression. Angiotensin II (Ang II) plays an important role in atherogenesis. Ang II increases macrophage cholesterol accumulation and foam cell formation, increases contraction of blood vessels and induces hypertrophy and hyperplasia of vascular smooth muscle cells (VSMC). Ang II significantly increases the expression of IL-6 mRNA and protein in vascular smooth muscle, in a dose-dependent manner. The induction of IL-6 expression by Ang II is dependent on intracellular Ca2+, tyrosine phosphorylation, and mitogen-activated protein kinase (MAPK) cxix. Ang II administration to apolipoprotein E-deficient atherosclerotic mice increases Ox-LDL degradation, CD36 mRNA expression, and CD36 protein expression by their peritoneal macrophages (MPMs). Ang II treatment of IL-6-deficient mice did not affect their MPM Ox-LDL uptake and CD36 protein levels. Furthermore, injection of IL-6 receptor antibodies in mice during Ang II treatment reduced macrophage Ox-LDL uptake and CD36 expression cxx.

Enzymatic, nonoxidative modification transforms low density lipoprotein (LDL) to an atherogenic molecule (E-LDL) that activates complement and macrophages and is present in early atherosclerotic lesions. E-LDL accumulates in human vascular smooth muscle cells (VSMC), where it stimulates the expression of gp130, the signal-transducing chain of the IL-6 receptor (IL-6R) family, and the secretion of Interleukin-6cxxi. IL-6/sIL-6R provokes marked up-regulation of gp130 mRNA and surface protein expression in VSMC. This is accompanied by secretion of IL-6 by the cells, so that an autocrine stimulation loop is created. In the wake of this self-sustaining system, there is a selective induction and secretion of monocyte chemotactic protein-1 (MCP-1), up-regulation of ICAM-1, and marked vascular smooth muscle proliferationcxxii. Interleukin-6 (IL-6) induces proliferation of vascular smooth muscle cells and the release of monocyte chemoattractant protein-1 (MCP-1). In one study, treatment with IL-6 caused rapid increase in the c-myc mRNA level of cultured vascular smooth muscle cells. IL-6 also stimulated DNA synthesis and proliferation of the cells significantly and dose-dependently at concentrations of more than 10 U/ml. The authors concluded that IL-6 may be important in the proliferation of VSMC, which is a key event in the development of atherosclerosiscxxiii. Another study investigated IL-6 mRNA expression in atherosclerotic arteries from patients undergoing surgical vascularization, utilizing reverse transcription polymerase chain reaction (RT-PCR) and in situ hybridization analyses. In RT-PCR analysis, the atherosclerotic arteries showed 10- to 40-fold levels of IL-6 mRNA expression over the non-atherosclerotic artery. In in-situ hybridization analysis, IL-6 gene transcripts were observed in the thickened intimal layer of atherosclerotic lesions. These results strongly suggest the involvement of IL-6 in the development of human atherosclerosiscxxiv. Thrombin is a potent mitogen for vascular smooth muscle cells (VSMCs) and plays an important role in the progression of atherosclerosis. Thrombin induces IL-6 mRNA and protein expression in a dose-dependent manner. Pharmacological inhibition of extracellular signal-regulated protein kinase (ERK), p38 mitogen-activated protein kinase (MAPK), or epidermal growth factor receptor (EGF-R) suppresses thrombin-induced IL-6 expressioncxxv. IL-6 increases the number of platelets in the circulationcxxvi and activates platelets through arachidonic acid metabolism in vitrocxxvii IL-6 is reported to increase plasma fibrinogen and decrease free protein S concentration. These IL-6-induced modifications of platelet and the coagulant phase of the clotting mechanism may lead to pathological thrombosis and instability of plaquecxxviii. IL-6 stimulation of vascular smooth muscle cells occurs via the JAK/STAT signaling pathway. In one study, Rat VSMC were stimulated with IL-6 in the presence or absence of a JAK 2 inhibitor, and the activation of STAT 3 (by Western), MCP-1 (by ELISA) and DNA synthesis (by (3)H-thymidine incorporation) was determined. IL-6 rapidly induced phosphorylation of STAT 3 in a dose- and time-dependent manner with a peak expression at 30 min. IL-6 also stimulated MCP-1 protein production and DNA synthesis dose dependently. 50 microM of AG490, a specific JAK 2 inhibitor, partially inhibited STAT 3 activation and MCP-1 production, with near complete inhibition of DNA synthesis. The authors concluded that the JAK/STAT pathway partially mediates IL-6-induced MCP-1 production and DNA synthesis in rat VSMC. The researchers further stated that these studies implicate a role of the JAK/STAT pathway in the development of vascular disease and atherosclerosiscxxix. Levels of IL-6 are significantly higher in patients with dyslipidemia IIa and IIb biochemically confirmed, and IL-6 levels are significantly correlated to intima-media complex thicknesscxxx.

STATINS AND INTERLEUKIN 6 The ability of HMG-CoA reductase inhibitors to lower C-reactive protein levels has recently brought into question the mechanisms of action of the statin drugs. Because these medications lower incidences of acute cardiovascular events as well as decreasing morbidity and mortality well before the effects of lowered LDL cholesterol can be expected to occur, questions have been asked about whether they may work independently of LDL-lowering mechanisms. One study examined the effects of atorvastatin on soluble adhesion molecules, interleukin-6 (IL-6) and brachial artery endothelial-dependent flow mediated dilatation (FMD) in patients with familial (FH) and non-familial hypercholesterolemia (NFH)cxxxi. A total of 74 patients (27 FH and 47 NFH) were recruited. Fasting lipid profiles, soluble intercellular adhesion molecule-1 (sICAM-1), soluble vascular-cellular adhesion molecule-1 (sVCAM-1), E-selectin, IL-6 and FMD were measured at baseline, 2 weeks, 3 and 9 months post-atorvastatin treatment (FH-80 mg/day, NFH-10 mg/day). In both groups, compared to baseline, sICAM-1 levels were significantly reduced at 2 weeks, further reduced at 3 months and maintained at 9 months (P<0.0001). The IL-6 levels were significantly reduced at 3 months and 9 months compared to baseline for FH(P<0.005) and NFH(P<0.0001). In both groups, the FMD at 2 weeks was higher than baseline (P<0.005), with progressive improvement up to 9 months. FMD was negatively correlated with sICAM-1 and IL-6. The authors concluded that both low and high doses of atorvastatin lead to early progressive improvement in endothelial function in patients with primary hypercholesterolemia and that sICAM-1 and IL-6 levels reflect endothelial dysfunction in these patients.

BISPHOSPHONATES AND INTERLEUKIN 6 Because of various modes of action observed in studies, bisphosphonates have been classified into two groups. Bisphosphonates (such as clodronate and etidronate) that closely resemble pyrophosphate—a normal byproduct of human metabolism—are incorporated into adenosine triphosphate (ATP) analogues, which create compounds that are believed to build up and lead to osteoclast death cxxxii. The newest generation of bisphosphonates, which contain nitrogen (such as pamidronate, alendronate, risedronate, and ibandronate), are believed to inhibit protein prenylation (post-translational modification) within the mevalonate pathway. The mevalonate pathway is responsible for the biosynthesis of cholesterol, other sterols, and isoprenoid lipids. Isoprenoid lipids are key in the prenylation of intracellular signaling proteins (GTPases) that, when activated, regulate a number of processes, including osteoclast activity. It is believed that by impeding the function of these regulatory proteins, bisphosphonates block osteoclast functioning and cause apoptosiscxxxiii

In patients with Paget's disease of bone, bisphosphonate therapy is associated with a significant reduction of Interleukin-6 soluble receptor (sIL-6R) serum levelscxxxiv. Bisphosphonates inhibit the production of pro-inflammatory cytokine interleukin-6 in tumoral cell lines of human osteoblastic phenotype (MG63 and SaOs cells), and in peripheral blood mononuclear cells (PBMC)cxxxv. Bisphosphonates also inhibit IL-1 and TNF-alpha stimulated IL-6 release in cultures of human osteoblastic osteosarcoma cellscxxxvi. Osteoblasts exposed to small amounts of bisphosphonate elaborate a soluble inhibitor, which interferes with osteoclast formation and developmentcxxxvii. Bisphosphonates prevent apoptosis of murine osteocytic ML0-Y4 cells, whether it is induced by etoposide, TNF-alpha, or glucocorticoid dexamethasonecxxxviii. Pamidronate and other bisphosphonates inhibit the production by osteoblasts of the inflammatory cytokine interleukin-6, a growth factor essential to myeloma cellscxxxix.

PLANT POLYPHENOLS, FATTY ACIDS AND INTERLEUKIN 6 The beneficial skeletal effects of genistein, at dietarily achievable levels, are mediated, by Interleukin-6. Interleukin-6 production was decreased 40% to 60% in osteoblastic cells treated with genistein from either day 8-16 or day 12-16, at dietarily achievable concentrations (10(−10) to 10(−8) M) (P<0.05)cxl. In one study, Sophoricoside (SOP) an isoflavone glycosid isolated from immature fruits of Sophora japonica (Leguminosae family) inhibited the interleukin (IL)-6 bioactivity with an IC50 value of 6.1 microMcxli. In another study, treatment with soybean isoflavones (10(−5)M), in the presence of TNF-alpha (10(−10)M), for 48 h inhibited production of IL-6 and PGE(2). The authors suggested that the antiresorptive action of soy phytoestrogen may be mediated by decreases in these local factorscxlii. One study investigated the mechanisms of drug resistance associated with the human prostate carcinoma PC-3 cell line. Endogenous and exogenous IL-6 and exogenous OM up-regulated cell growth and enhanced resistance of PC-3 tumor cells to both etoposide and cisplatin. Both IL-6- and OM-mediated effects were inhibited by the treatment of PC-3 with an antisense oligodeoxynucleotide against gp130, the protein kinase inhibitor genistein (GNS), or the monoterpene perillic acid (PA), a posttranslational inhibitor of p21ras isoprenylationcxliii. In another study, the effect of inhibition of tyrosine kinase activity on thymidine uptake into cultured human pituitary adenoma cells was studied using two inhibitors, genestein and methyl-2,3-dihydroxycinnamate (MDHC). Of 33 pituitary adenomas, 7 incorporated sufficient [3H]thymidine to be investigated in the experiments. Genestein and MDHC both potently inhibited thymidine uptake into these tumors, with a mean inhibition by 74 mmol/L genestein of 61.96+/−18.96% (+/−SD inhibition of basal), by 740 mmol/L genestein of 92.65+/−8.59%, and by 100 mmol/L MDHC of 93.84+/−3.85%. Epidermal growth factor stimulated thymidine uptake in 2 of the 3 clinically nonfunctioning adenomas studied, and this stimulation was inhibited by genestein. The authors concluded that tyrosine kinase activity is crucial for the integrity and growth of pituitary adenomas in culture and that growth factors released by pituitary adenomas potentially may maintain and promote tumor growth by stimulating tyrosine kinase activitycxliv.

Bacterial LPS induce a 12- to 16-fold increase in IL-1 beta, IL-6, and TNF-alpha mRNA levels. In one study, this increase was completely or more than 80% blocked by the protein tyrosine kinase specific inhibitors herbimycin A and genistein at the concentrations of 1.7 and 37 microM, respectively. LPS-induced IL-6 protein synthesis and IL-6 bioactivity were also reduced to baseline levels by the PTK inhibitors herbimycin A and genistein. Both PTK inhibitors also reduced the LPS activation of nuclear factor-kappa B (NF-kappa B), which is a transcription factor involved in the expression of cytokine genes such as IL-6 and TNF-alpha.cxlv

Epidemiological evidence suggests that tea consumption may have a strong effect on cardiovascular disease, but there has been no prior description of the molecular mechanisms involved. Epigallocatechin-3-gallate (EGCG) is a prominent catechin present in green tea. Several experimental studies have reported beneficial effects of EGCG in inflammation and cancercxlvi cxlvii cxlviii. NF-κB, is a transcription factor centrally involved in the signal transduction of the inflammatory process. The common pathway for activation of NF-κB involves phosphorylation of its inhibitor protein IκB-α by IKK. Activation of IKK complex is an essential step for NF-κB activation because the kinase phosphorylates IκB-α and allow its degradation. Several studies have demonstrated that EGCG is an effective inhibitor of IKK activity. EGCG inhibits TNF-α-mediated IKK activation in human epithelial cells. Yang and colleagues showed that EGCG in concentrations of 50 to 200 μM inhibited IKK activity in an intestinal epithelial cell linecxlix. In the Myocardial ischemia reperfusion study, EGCG reduced reperfusion-induced activation of IKK, degradation of IκB-α, and activation of NF-κBcl. EGCG has been demonstrated to dramatically inhibit chemokine induced neutrophil chemotaxis in vitrocli. Tea polyphenols have also been noted to induce apoptosis and cell cycle arrest in a wide array of cell lines clii cliii cliv. EGCG affects several signaling mechanisms in inflammation. Menegazzi and colleagues showed that interferon-γ-induced STAT-1 activation in carcinoma-derived cell lines of non-gut origin was blocked by EGCGclv. In another study, Watson and colleagues demonstrated that EGCG significantly reduced INF-γ-induced STAT1 activation in T84 epithelial and THP-1 monocytes/macrophagesclvi.

In vitro studies have demonstrated that cellular targets of EGCG that may account for its anti-inflammatory properties include protein kinase C clvii clviii, activation of extracellular mitogen-activated protein kinases clix, and STAT-iclx. EGCG is a potent inhibitor of IL-8 gene expression in human respiratory epithelial cells. The proximal mechanism of this effect involves, in part, inhibition of IKKclxi. In one study, the effects of EGCG in myocardial reperfusion injury were examined. Male Wistar rats were subjected to myocardial ischemia (30 min) and reperfusion (up to 2 h). Rats were treated with EGCG (10 mg/kg intravenously) or with vehicle at the end of the ischemia period followed by a continuous infusion (EGCG 10 mg/kg/h) during the reperfusion period. In vehicle-treated rats, extensive myocardial injury was associated with tissue neutrophil infiltration as evaluated by myeloperoxidase activity, and elevated levels of plasma creatine phosphokinase. Vehicle-treated rats also demonstrated increased plasma levels of interleukin-6. These events were associated with cytosol degradation of inhibitor κB-α, activation of IκB kinase, increased phosphorylation of c-Jun in a time-dependent manner, and subsequent activation of nuclear factor-κB and activator protein-1 in the infarcted heart. In vivo treatment with EGCG markedly attenuated phosphorylation of c-Jun at all time points, reduced myocardial damage and myeloperoxidase activity. Plasma IL-6 and creatine phosphokinase levels were decreased after EGCG administration. This beneficial effect of EGCG was associated with reduction of nuclear factor-KB and activator protein-1 DNA bindingclxii. In another study, the capacity of the flavan-3-ols [(−)-epicatechin (EC) and (+)-catechin (CT)] and a B dimeric procyanidin (DP-B) to modulate phorbol 12-myristate 13-acetate (PMA)-induced NF-kappaB activation in Jurkat T cells was investigated. The classic PMA-triggered increase in cell oxidants was prevented when cells were preincubated for 24 h with EC, CT, or DP-B (1.7-17.2 microM). PMA induced the phosphorylation of IKKbeta and the subsequent degradation of IkappaBalpha. These events were inhibited in cells pretreated with the flavonoids. PMA induced a 4.6-fold increase in NF-kappaB nuclear binding activity in control cells. Pretreatment with EC, CT, or DP-B decreased PMA-induced NF-kappaB binding activity and the transactivation of the NF-kappaB-driven gene IL-2clxiii.

In a research study, the effects of the green tea catechin EGCG in myocardial reperfusion injury were examined. Male Wistar rats were subjected to myocardial ischemia (30 min) and reperfusion (up to 2 h). Rats were treated with EGCG (10 mg/kg intravenously) or with vehicle at the end of the ischemia period followed by a continuous infusion (EGCG 10 mg/kg/h) during the reperfusion period. In vehicle-treated rats, extensive myocardial injury was associated with tissue neutrophil infiltration as evaluated by myeloperoxidase activity, and elevated levels of plasma creatine phosphokinase.

Vehicle-treated rats also demonstrated increased plasma levels of interleukin-6. These events were associated with cytosol degradation of inhibitor κB-α, activation of IκB kinase, increased phosphorylation of c-Jun in a time-dependent manner, and subsequent activation of nuclear factor-KB and activator protein-1 in the infarcted heart. In vivo treatment with EGCG markedly attenuated phosphorylation of c-Jun at all time points, reduced myocardial damage and myeloperoxidase activity. Plasma IL-6 and creatine phosphokinase levels were decreased after EGCG administration. This beneficial effect of EGCG was associated with reduction of nuclear factor-KB and activator protein-1 DNA bindingclxiv. Another study investigated the effect of a polyphenol rich extract from black tea and vitamin E on bacterial lipopolysaccharide (endotoxin) induced IL-6 production, alterations in liver glutathione and antioxidant acute phase protein (caeruloplasmin) concentration, in rats fed on a synthetic diet for 21 days. In the vitamin E sufficient group a significantly lower IL-6 concentration than in vitamin E deficient animals was observed. Addition of tea extract to the diet produced a similar reduction in IL-6.clxv

Polyunsaturated omega-3 fatty acids reduce the secretion of proinflammatory cytokines and downregulate the inflammatory process. 18-week n-3 PUFA diet supplementation exerts a significant inhibitory effect on basal and lipopolysaccharide (LPS)-stimulated IL-6 monocyte production (50% and 46%, respectively, P<0.05)clxvi.

One study examined the effects of polyunsaturated fatty acids (PUFAs) on the production of IL-6 by human unstimulated endothelial cells (EC) or EC stimulated with TNF-alpha (100 U/ml); IL-4 (100 U/ml); LPS (1 ug/ml); or allogeneic peripheral blood lymphocytes (PBL). Twenty-four hour culture supernatants of immunoreactive IL-6 were measured by Sandwich ELISA. The production of IL-6 was potentiated when EC were stimulated with TNF-alpha; IL-4; LPS; or monocyte-depleted PBL in comparison to unstimulated EC. The addition of n-3 PUFAs in culture medium (100 ug/ml DHA or EPA) significantly reduced the production of IL-6 by unstimulated EC; or stimulated with TNF-alpha; IL-4 pg/ml); LPS or depleted PBL respectively for DHA and EPA, whereas the n-6 PUFAs (Arachidonic acid), even used at the highest concentration, was ineffective. The researchers concluded that this inhibitory effect is PUFA dose dependent but is more potent with EPA than DHAclxvii Another study investigated the effects of a low n-6 fatty acid (FA) diet supplemented with fish oil on serum pro-inflammatory cytokine concentrations and clinical variables in patients with active rheumatoid arthritis (RA). Sixty patients were randomly assigned to receive a diet low in n-6 FAs and n-3 FAs supplement (fish oil group), a diet low in n-6 FAs and placebo (placebo group), or no special diet or intervention (control group). Serum cytokines and clinical and biochemical variables were evaluated at baseline and various timepoints. At week 18 the fish oil group had significant reductions in linoleic acid, C-reactive protein (CRP) and soluble tumour necrosis factor receptor p55 (sTNF-R p55), and significant elevations in eicosapentaenoic acid and docosahexaenoic acid compared with baseline. There were no significant differences in the clinical variables between the three groups. At week 24 there were significant reductions in interleukin-6 and TNF-alpha in the fish oil and placebo/low n-6 FA groupsclxviii. In another study, an isocaloric exchange of ALA for LA was found to reduce fasting sIL6R concentration by approximately 11% after a four-day dietary intervention in both overweight/obese and normal weight subjectsclxix.

ATHEROSCLEROSIS AND STATINS Changes in intima-media thickness (IMT) and arterial lumen diameter-as measured by B-mode high-resolution ultrasonography and quantitative coronary angiography, respectively-are currently the only surrogate markers for progression of atherosclerotic disease recognized by regulatory authorities in the United States and Europe. Because atherosclerosis is a disease of the arterial wall, the ability of B-mode ultrasonography to provide visualization of IMT offers significant advantages over angiography. These advantages, as well as the safety and noninvasiveness of B-mode ultrasonography, have led to increasing use of this imaging technique in observational studies and interventional studies of lipid-lowering agents over the last decade. These observational studies clearly demonstrated an association between carotid IMT and atherosclerotic disease. Of the interventional studies, the recent Arterial Biology for the Investigation of the Treatment Effects of Reducing Cholesterol (ARBITER) trial found that use of atorvastatin 80 mg daily for aggressive lowering of plasma low-density lipoprotein cholesterol (LDL-C) concentrations to below current target levels was associated with significant IMT regression compared with results obtained with less aggressive plasma LDL-C lowering.clxxclxxi

ATHEROSCLEROSIS AND BISPHOSPHONATES Measurement of carotid arterial intima-media thickness (IMT) using B-mode ultrasonography is a noninvasive and powerful tool to evaluate early atherosclerotic lesionsclxxii clxxiii clxxiv clxxv clxxvi. In one study the effect of etidronate treatment on carotid arterial intima-media thickness was prospectively examined in 57 subjects with type 2 diabetes associated with osteopenia. After 1 yr of therapy with cyclical etidronate (200 mg/day for 2 weeks every 3 months), intima-media thickness showed a decrease (mean±SE, −0.038±0.011 mm), which was significantly different from a change in 57 control subjects (0.023±0.015 mm; P<0.005). Cardiovascular parameters were not changed after etidronate treatment. The authors concluded that etidronate in clinical dosage may have an antiatherogenic action, at least in type 2 diabetes, although its mechanisms remain to be elucidatedclxxvii. In another study, administration of ethane-1-hydroxy-1,1-diphosphonate (EHDP) to swine with pre-established atherosclerosis resulted in lower lesion calcium concentration, smaller lesions and a decrease in the area of lesions involved in necrosis. Atherosclerosis was developed in Yorkshire swine by balloon catheter-injury to the abdominal aorta, followed by a high cholesterol-high lipid (HL) diet for 4 months. The administration of EHDP (20 mg/kg/day) was begun after these 4 months and continued for 5 additional months along with the atherogenic diet. Other swine were ballooned and fed HL diet for nine months. Morphometric analysis showed that the extent of lesions, expressed as ratio of intima to media was significantly less (P less than 0.05) in the EHDP-treated HL swine, compared to the HL diet-only group. The ratio of lesion areas showing lipid-rich necrotic debris to the area of media was also significantly smaller (P less than 0.05). Biochemical analysis showed that the lesion from the HL drug-treated group contained significantly less (P less than 0.05) calcium compared to that from the HL diet only. Finally, there was significant correlation between average lesion area and average lesion calcium concentration (P less than 0.02) for both groups. While the effect of EHDP on lesion size and calcium concentration has been previously reported for various species such as rabbit and monkey, the authors concluded that this study is believed to be the first where a beneficial effect of EHDP on one of the most serious complications of atherogenesis—necrosis—has been documentedclxxviii.

ATHEROSCLEROSIS, PLANT POLYPHENOLS AND FATTY ACIDS Cupric-ion-oxidized LDL (CuLDL) or endothelial cell-oxidized LDL (ELDL) induces the activation by Tyr-phosphorylation of JAK2, one of the Janus kinase involved upstream of STATs in the JAK/STAT pathway of cytokine transduction. Oxidized LDL (OxLDL) also initiates STAT1 and STAT3 Tyr-phosphorylation and translocation to the nucleus, with a more marked effect for the extensively modified CuLDL. In one study, Genistein, a nonspecific Tyr-kinase inhibitor, and AG490, a specific inhibitor of JAKs, markedly prevented the CuLDL-induced enhancement of STAT1 and STAT3 Tyr-phosphorylation and DNA-binding activity, suggesting that JAKs are the main kinases involved in STATs' activation by oxidized LDLclxxix. The effect of genistein on aortic atherosclerosis was studied in New Zealand White rabbits. After provocation of atherosclerosis with hyperlipidemic diet, the rabbits were divided as hyperlipidemic diet group (HD), normal diet group (ND) and hyperlipidemic plus genistein diet group (HD+genistein) for 4 and half months. The average cross sectional area of atherosclerotic lesion was 0.269 mm2 after provocation. The lesion was progressed by continuous hyperlipidemic diet (10.06 mm2) but was increased mildly by genistein (0.997 mm2), and decreased by normal diet. The ratio of macrophages to smooth muscle cells in the lesion was not changed by genistein supplementation. The western blotting showed reduction of MMP-3 expression in HD+genistein and ND groups than HD groupclxxx. Angiotensin II (Ang II) plays an important role in atherogenesis. One study investigated the effect of Ang II on the production of interleukin-6 (IL-6) in rat vascular smooth muscle cells. Ang II significantly increased the expression of IL-6 mRNA and protein in a dose-dependent manner (10(−10) to 10(−6) mol/L). The expression of IL-6 mRNA induced by Ang II was completely blocked by an Ang II type 1 receptor antagonist, CV11974. Inhibition of tyrosine kinase with genistein, and inhibition of mitogen-activated protein kinase with PD98059 completely abolished the effect of Ang II.clxxxi. The potent endothelium-derived vasoactive factor endothelin-1 (ET-1) has been implicated in the pathophysiology of atherosclerosis and its complications. ET-1 stimulates the formation of proinflammatory cytokines including Interleukin-6 and tumor necrosis factor alpha (TNF alpha)clxxxii. In one study ET-1 transiently increased IL-6 mRNA compatible with regulation of IL-6 release at the pretranslational level. Electrophoretic mobility shift assays demonstrated time- and concentration-dependent activation of the proinflammatory transcription factor nuclear factor-kappaB (NF-kappaB) in ET-1-stimulated human vascular SMC. A decoy oligodeoxynucleotide bearing the NF-kappaB binding site inhibited ET-1-stimulated IL-6 release to a great extent suggesting that this transcription factor plays a key role for cytokine production elicited by ET-1clxxxiii.

In one study, researchers investigated the suppressive effect of cocoa powder (cacao polyphenol content: 7.8%) on atherosclerosis in a spontaneous familial hypercholesterolemic model, Kurosawa and Kusanagi-hypercholesterolemic (KHC) rabbits. Six-month dietary administration of cocoa powder had no effects on body weight, hematology or blood chemistry parameters or a lipid profile in KHC rabbits. Thiobarbituric acid reactive substances (TBARS), the marker of lipid peroxidation, in plasma were decreased in the cocoa powder treated group from the 2nd month of administration during the study period compared to that in the control group. The area of atherosclerotic lesions in the aorta was significantly smaller in the cocoa powder group (30.87%) than in the control (52.39%). Tissue cholesterol content also tended to decrease. Distensibility of the aortic wall was improved significantly in the cocoa powder treated group due to decreases in fatty streaks and intimal thickening compared to that in the control group. These results suggest that cocoa powder has suppressive effect on development of atherosclerotic lesionsclxxxiv. One study determined the effects of green tea polyphenols on the proliferation and p44/42 mitogen-activated protein kinase (MAPK) activity in rat VSMCs simulated by native LDL. Rat aortic VSMCs were cultured and treated with LDL (100 microg/ml) in the absence or presence of green tea polyphenols, and the cell proliferation was subsequently quantified by non-radioactive MTS/PES assay and the cell cycle analyzed by flow cytometry. The p44/42 MAPK activity was evaluated by immunoblotting using anti-p44/42 phospho-MAPK antibody. Compared with the cells without polyphenol treatment, the proliferation of the VSMCs induced by LDL was dose-dependently inhibited by green tea polyphenols (P<0.05), with more numerous cells in G(O)G(1) phase (P<0.05) as shown by flow cytometry analysis. LDL significantly enhanced the p44/42 MAPK activity, an effect obviously inhibited by green tea polyphenols (at 100 microg/ml). These results suggest that green tea polyphenols can inhibit high levels of LDL-induced proliferation of phosphorylated p44/42 MAPK expression in rat VSMCsclxxxv. In another study, hamsters (nine in each group) were given a cholesterol/saturated fat for 10 weeks to induce foam cell formation. Water or 6.75% ethanol was given to the control groups. Beverages tested included red wine, dealcoholized red wine, and red grape juice, all diluted in half. Ethanol and all beverages caused a significant reduction in atherosclerosis. The combination of ethanol in red wine had the largest effect in decreasing atherosclerosis. When compared with dealcoholized wine and normalized to polyphenol dose, red wine's beneficial effects can be attributed entirely to the polyphenols. Grape juice had a significant benefit at a much lower dose of polyphenols than the wines. Grape juice was calculated to be much more effective than red wine or dealcoholized red wine at the same polyphenol dose in inhibiting atherosclerosis and improving lipids and antioxidant parameters. The authors suggest that polyphenolic beverages from grapes are beneficial in inhibiting atherosclerosis by several mechanismsclxxxvi.

The low incidence of cardiovascular disease associated epidemiologically with high consumption of food rich in n-3 fatty acids suggests the possibility that part of the beneficial cardiovascular effects of these natural substances may be due to a reduction of atherosclerosisclxxxvii. Dietary intervention trials using coronary heart disease (CHD) mortality and morbidity as endpoints have demonstrated that restriction of dietary total and saturated fat or replacement of the latter with polyunsaturated fatty acids (PUFAs), in particular n-3 PUFAs, is of great benefit with respect to CHD riskclxxxviii. A range of prospective studies have proven high efficacy of omega3-polyunsaturated fatty acids in secondary prophylaxis of atherosclerosis and its complicationsclxxxix One study investigated the relationship between intake of N-3 fatty acids and carotid atherosclerosis in the cross-sectional study. A total of 1920 Japanese, aged over 40 years, received a population-based health examination in 1999. They underwent B-mode carotid ultrasonography to evaluate the carotid intimal-medial thickness (IMT). Eating patterns were evaluated by a 105 items food frequency questionnaire. A complete data set was available for 1902 subjects (785 men and 1117 women). The mean eicosapentaenoic acid (EPA) intake in men was 0.32+/−0.23 g/day and in women was 0.31+/−0.20 g/day. The mean docosahexaenoic acid (DHA) intake in men was 0.52+/−0.34 g/day and in women was 0.49+/−0.29 g/day. With multiple linear regression analysis, after adjustments for age, sex, and total energy intake, intakes of EPA (P<0.05), DHA (P<0.05), and docosapentaenoic acid (P<0.05) were significantly and inversely related to IMT. These data indicate that dietary N-3 fatty acid, especially very long chain N-3 fatty acids, may protect against carotid atherosclerosiscxc.

Type 2 Diabetes and Interleukin 6

Circulating levels of interleukin-6 (IL-6) are raised in insulin resistant states such as obesity, impaired glucose tolerance (IGT), and type 2 diabetes mellitus (DM). Growing evidence suggests that IL-6 is not only produced by fat cells but is also capable of inducing insulin resistance in these cells. The expected result of this in vivo, would be to increase adipose mass and subsequently body mass index (BMI). The IL-6-174G>C common functional gene variant has consistently been associated with increased plasma IL-6, insulin resistance, and increased cardiovascular risk.cxci. In another study, the authors determined whether elevated levels of the inflammatory markers interleukin 6 (IL-6) and C-reactive protein (CRP) are associated with development of type 2 DM in healthy middle-aged women. The Women's Health Study, is an ongoing US primary prevention, randomized clinical trial initiated in 1992. From a nationwide cohort of 27 628 women free of diagnosed DM, cardiovascular disease, and cancer at baseline, 188 women who developed diagnosed DM over a 4-year follow-up period were defined as cases and matched by age and fasting status with 362 disease-free controls. Incidence of confirmed clinically diagnosed type 2 DM by baseline levels of IL-6 and CRP. Study results showed that baseline levels of IL-6 (P<0.001) and CRP(P<0.001) were significantly higher among cases than among controls. The relative risks of future DM for women in the highest vs lowest quartile of these inflammatory markers were 7.5 for IL-6 (95% confidence interval [CI], 3.7-15.4) and 15.7 for CRP (95% CI, 6.5-37.9). Positive associations persisted after adjustment for body mass index, family history of diabetes, smoking, exercise, use of alcohol, and hormone replacement therapy; multivariate relative risks for the highest vs lowest quartiles were 2.3 for IL-6 (95% CI, 0.9-5.6; P for trend=0.07) and 4.2 for CRP (95% CI, 1.5-12.0; P for trend=0.001). Similar results were observed in analyses limited to women with a baseline hemoglobin A(1c) of 6.0% or less and after adjustment for fasting insulin level. The authors concluded that elevated levels of CRP and IL-6 predict the development of type 2 DM, and the data support a possible role for inflammation in diabetogenesis.

TYPE 2 DIABETES AND BISPHOSPHONATES Advanced glycation end products (AGE), senescent macroprotein derivatives form at an accelerated rate in diabetes and induce angiogenesis through overgeneration of autocrine vascular endothelial growth factor (VEGF). In this study, effects of incadronate disodium, a nitrogen-containing bisphosphonate on AGE-elicited angiogenesis in vitro, were studied. Incadronate disodium was found to completely inhibit AGE-induced increase in DNA synthesis as well as tube formation of human microvascular endothelial cells (EC). Furthermore, incadronate disodium significantly prevented transcriptional activation of nuclear factor-kappaB and activator protein-1 and the subsequent up-regulation of VEGF mRNA levels in AGE-exposed EC. Farnesyl pyrophosphate, but not geranylgeranyl pyrophosphate, was found to completely reverse the anti-angiogenic effects of incadronate disodium on EC. These results suggest that incadronate disodium could block the AGE-signaling pathway in microvascular EC through inhibition of protein farnesylation. The authors concluded that Incadronate disodium may be a promising remedy for treatment of patients with proliferative diabetic retinopathycxcii cxciii. Charcot neuroarthropathy has been recognized for over 130 years and yet it remains a major cause of morbidity for patients with diabetes mellitus and a continuing challenge for physicians. The underlying cause is thought to be trauma in a neuropathic foot that leads to a complex series of pathological processes culminating in bone and joint destruction and subsequent deformity. A study was undertaken to study the effect of pamidronate, a bisphosphonate, in the management of acute diabetic Charcot neuroarthropathy. Altogether 39 diabetic patients with active Charcot neuroarthropathy from four centers in England were randomized in a double-blind placebo-controlled trial. Patients received a single infusion of 90 mg of pamidronate or placebo (saline). Foot temperatures, symptoms and markers of bone turnover (bone specific alkaline phosphatase and deoxypyridinoline crosslinks) were measured over the 12 months, in 10 visits. All patients also had standard treatment of the Charcot foot. Mean age of the study group (59% Type 2 (non-insulin-dependent) diabetes mellitus) was 56.3+/−10.2 years. The mean temperature difference between active and control groups was 3.6+/−1.7 degrees C. and 3.3+/−1.4 degrees C., respectively. There was a fall in temperature of the affected foot in both groups after 2 weeks with a further reduction in temperature in the active group at 4 weeks (active and placebo vs baseline; p=0.001; p=0.01, respectively), but no difference was seen between groups. An improvement in symptoms was seen in the active group compared with the placebo group (p<0.001). Reduction in bone turnover (means+/−SEM) was greater in the active than in the control group. Urinary deoxypyridinoline in the pamidronate treated group fell to 4.4+/−0.4 nmol/mmol creatinine at 4 weeks compared with 7.1+/−1.0 in the placebo group (p=0.01) and bone-specific alkaline phosphatase fell to 14.1+/−1.2 u/l compared with 18.6+/−1.6 u/l after 4 weeks, respectively (p=0.03). The authors concluded that the bisphosphonate, pamidronate, given as a single dose leads to a reduction in bone turnover, symptoms and disease activity in diabetic patients with active Charcot neuroarthropathycxciv.

TYPE II DIABETES AND STATINS In West of Scotland Coronary Prevention Study (WOSCOPS)cxcv, development of type 2 diabetes mellitus (DM) was found to decrease by 30% in pravastatin-treated patients. One study investigated the effects of an HMG-CoA reductase inhibitor, atorvastatin, on insulin sensitization in performed in chow fed Zucker lean and fatty rats treated with atorvastatin 50 mg/kg/day (ATORVA-50) and results were compared to Zucker lean and fatty rats treated with drug vehicle only (CONT). Treatment with atorvastatin resulted in a dose-dependent improvement in whole body insulin sensitivity in both lean and fatty rats, with an approximately two-fold increase in glucose infusion rate and glucose disposal (Rd) in ATORVA-50 versus CONT (p<0.01)cxcvi. Another study investigated the effects of atorvastatin on the glucose metabolism and insulin resistance of KK/Ay mice, an animal model of type II diabetes, were investigated. Atorvastatin significantly decreased the non-HDL-cholesterol level in the oral glucose tolerance test, inhibited increase in the 30-min glucose level, decreased plasma insulin levels before and 30 and 60 minutes after glucose loading, and decreased the insulin resistance index, compared with corresponding values in controls, indicating that atorvastatin appeared to improve glucose metabolism by improving insulin resistancecxcvii.

Type 2 diabetes is associated with a substantially increased risk of cardiovascular disease, but the role of lipid-lowering therapy with statins for the primary prevention of cardiovascular disease in diabetes is inadequately defined. One study aimed to assess the effectiveness of atorvastatin 10 mg daily for primary prevention of major cardiovascular events in patients with type 2 diabetes without high concentrations of LDL-cholesterol. 2838 patients aged 40-75 years in 132 centers in the UK and Ireland were randomized to placebo (n=1410) or atorvastatin 10 mg daily (n=1428). Study entrants had no documented previous history of cardiovascular disease, an LDL-cholesterol concentration of 4.14 mmol/L or lower, a fasting triglyceride amount of 6.78 mmol/L or less, and at least one of the following: retinopathy, albuminuria, current smoking, or hypertension. The primary endpoint was time to first occurrence of the following: acute coronary heart disease events, coronary re-vascularisation, or stroke. Analysis was by intention to treat. The trial was terminated 2 years earlier than expected because the pre-specified early stopping rule for efficacy had been met. Median duration of follow-up was 3.9 years (IQR 3.0-4.7). 127 patients allocated placebo (2.46 per 100 person-years at risk) and 83 allocated atorvastatin (1.54 per 100 person-years at risk) had at least one major cardiovascular event (rate reduction 37% [95% CI −52 to −17], p=0.001). Treatment would be expected to prevent at least 37 major vascular events per 1000 such people treated for 4 years. Assessed separately, acute coronary heart disease events were reduced by 36% (−55 to −9), coronary re-vascularisations by 31% (−59 to 16), and rate of stroke by 48% (−69 to −11). Atorvastatin reduced the death rate by 27% (−48 to 1, p=0.059). No excess of adverse events was noted in the atorvastatin group. The study authors determined that Atorvastatin 10 mg daily is safe and efficacious in reducing the risk of first cardiovascular disease events, including stroke, in patients with type 2 diabetes without high LDL-cholesterol. The researchers stated that no justification is available for having a particular threshold level of LDL-cholesterol as the sole arbiter of which patients with type 2 diabetes should receive statins. The authors concluded that debate about whether all people with this disorder warrant statin treatment should now focus on whether any patients are at sufficiently low risk for this treatment to be withheldcxcviii.

TYPE II DIABETES, FOOD POLYPHENOLS AND FATTY ACIDS Nutritional intervention studies performed in animals and humans suggest that the ingestion of soy protein associated with isoflavones and flaxseed rich in lignans improves glucose control and insulin resistance. In animal models of obesity and diabetes, soy protein has been shown to reduce serum insulin and insulin resistance. In studies of human subjects with or without diabetes, soy protein also appears to moderate hyperglycemia and reduce body weight, hyperlipidemia, and hyperinsulinemia, supporting its beneficial effects on obesity and diabetescxcix. Recent studies have provided evidence that soy consumption alleviates some of the symptoms associated with Type 2 diabetes such as insulin resistance and glycemic control cc cci. Some of these effects may be the end result of the improved blood lipid profile caused by soy consumption.

Isoflavones may improve lipid and glucose metabolism by acting as an antidiabetic PPAR agonistccii Peroxisome-proliferator activated receptors (PPAR), are nuclear receptors that participate in cellular lipid homeostasis and insulin actioncciii cciv ccv. Upon ligand binding, PPAR are activated and bind to peroxisome-proliferator response element (PPRE) sequences located within the promoters of PPAR-regulated genes. Ligands for PPARγ include some unsaturated fatty acids and their derivatives as well as glitazones, insulin-sensitizing drugs used to treat Type 2 diabetes. Ligands for PPARα include some saturated and unsaturated fatty acids as well as the group of drugs termed fibrates, which are hypolipidemic agents used to manage elevated blood lipid levels and Type 2 diabetes. Generally, PPARα controls the transcription of many genes involved in lipid catabolism, whereas PPARγ controls the expression of genes involved in adipocyte differentiation and insulin sensitization. Peroxisome proliferator-activated receptor γ2 (PPARγ2) antagonizes the transcriptional activity of NF-kappaB. Together, activation of PPARα and PPARγ increases β-oxidation and insulin sensitization, whereas blood and liver lipid concentrations are typically reduced.

Obesity and insulin resistance are often associated with lower circulating adiponectin concentrations and elevated serum interleukin-6 (IL-6) and/or tumor necrosis factor-alpha (TNF-alpha). Adiponectin suppresses activation of nuclear factor-kappaB (NF-kappaB) in aortic endothelial cells and porcine macrophages. One study determined whether adiponectin alters Peroxisome proliferator-activated receptor γ2 (PPARγ2) expression in pig adipocytes. PPARγ2 antagonizes the transcriptional activity of NF-kappaB. Primary adipocytes from pig subcutaneous adipose tissue were treated with or without lipopolysaccharide (LPS; 10 microg/ml) and adiponectin (30 microg/ml), and nuclear extracts were obtained for gel shift assays to assess nuclear localization of NF-kappaB. Whereas LPS induced an increase in NF-kappaB activation, adiponectin suppressed both NF-kappaB activation and the induction of IL-6 expression by LPS(P<0.05). Similar results were obtained in 3T3-L1 adipocytes. Adiponectin also induced an upregulation of PPARgamma2 mRNA (P<0.05). Although interferon-gamma (IFN-gamma) did not reduce the basal expression of PPARgamma2, it suppressed PPARgamma2 induction by adiponectin (P<0.05).ccvi. One study determined the effects of genistein, a tyrosine kinase inhibitor, on retinal vascular permeability in an experimental diabetic rat model. Seventy-two rats were equally divided into four groups: (1) nondiabetic control group, (2) diabetic control group, (3) diabetic rats receiving 150 mg genistein/kg food, and (4) diabetic rats receiving 300 mg genistein/kg food. Diabetes was induced by streptozotocin injection in the three diabetic groups. Rats were fed diets with or without genistein and followed for 6 months. Retinal vascular permeability was assessed by measuring radiolabeled sucrose leakage into the retina and by Western blot analysis for total retinal albumin. Retinal phosphotyrosine levels and proliferating cell nuclear antigen (PCNA) were also evaluated by Western blot analysis. Diabetic control rats had markedly increased retinal vascular leakage of radiolabeled sucrose compared with nondiabetic control rats. Diabetic rats receiving oral genistein had significantly less retinal vascular leakage of radiolabeled sucrose than diabetic control rats in a dose-response fashion. Diabetic control rats had increased levels of phosphotyrosine, retinal albumin, and PCNA by Western blot analysis compared with nondiabetic control rats. Rats receiving 300 mg of genistein had decreased retinal albumin by Western blot analysis. Western blot analysis demonstrated a dose-response decrease in retinal phosphotyrosine levels and PCNA in genistein-treated diabetic rats compared with diabetic control rats. The authors concluded that long-term oral administration of genistein significantly inhibits retinal vascular leakage in experimentally induced diabetic rats. Tyrosine kinase inhibition may be a useful pharmacological approach for the treatment of diabetic-induced retinal vascular leakageccvii

The beta subunit of the signalsome—IKKbeta, a crucial catalyst of NF-kappaB activation—is an obligate mediator of the disruption of insulin signaling induced by excessive exposure of tissues to free fatty acids and by hypertrophy of adipocytes. IKKbeta plays a crucial role, not only in the induction of insulin resistance, but also atherogenesis, a host of inflammatory disorders, and the survival and spread of cancer. The polyphenols resveratrol and silibinin. inhibit or suppress the activation of IKKbetaccviii. In one study, water-soluble polyphenol polymers from cinnamon that increase insulin-dependent in vitro glucose metabolism roughly 20-fold and display antioxidant activity were isolated and characterized by nuclear magnetic resonance and mass spectroscopy. The polymers were composed of monomeric units with a molecular mass of 288. Two trimers with a molecular mass of 864 and a tetramer with a mass of 1152 were isolated. Their protonated molecular masses indicated that they are A type doubly linked procyanidin oligomers of the catechins and/or epicatechins. The authors concluded that the polyphenolic polymers found in cinnamon may function as antioxidants, potentiate insulin action, and may be beneficial in the control of glucose intolerance and diabetesccix. Epidemiologic studies have reported a lower prevalence of impaired glucose tolerance and type 2 diabetes in populations consuming large amounts of the n-3 long-chain polyunsaturated fatty acids (n-3 LC-PUFAs) found mainly in fish. In one study, the hypoglycemic and hypolipidemic effect of docosahexaenoic acid (DHA; C22: 6omega-3) ethyl ester was examined in KK-Ay mice and neonatal streptozotocin-induced diabetic (NSZ) which are respectively obese and lean animal models of non-insulin-dependent diabetes mellitus (NIDDM), and in ddY normal mice. Single administration of DHA (500 mg/kg body weight) to KK-Ay mice significantly reduced (p<0.05) the blood glucose levels (BG) (p<0.05) and plasma free fatty acid levels (FFA) (p<0.05) at 10 h after oral administration when compared with control group. DHA (500 mg/kg body weight)-treated NSZ and normal mice, however, showed no change in these parameters. In addition, repeated administration of DHA (100 mg/kg) to KK-Ay mice significantly suppressed the increment of BG (p<0.05) and plasma triglyceride levels (TG) (p<0.01), and significantly decreased FFA (p<0.05) at 30 d compared with control group. DHA also significantly decreased the blood glucose at 60 and 120 min on insulin tolerance test (ITT)ccx. Another study performed repeated experiments to test the effects of fish oil supplementation in Sprague-Dawley rats fed a fructose-rich diet (group F). Compared with control rats on a normal diet (group C), group F consistently developed hypertriglyceridemia without elevated plasma free fatty acid (FFA), fasting hyperinsulinemia together with fasting hyperglycemia (insulin resistance syndrome), and systolic hypertension within 3 weeks. Insulin-stimulated glucose uptake and insulin binding of adipocytes were significantly reduced. Rats fed the same high-fructose diet but supplemented with fish oil (group O) had alleviation of all of these metabolic defects and a normalized insulin sensitivity and blood pressure. Beta-Cell function as shown by plasma glucose and insulin responses to oral glucose remained intact in group F and group Occxi.

OSTEOPOROSIS AND INTERLEUKIN 6 Osteoporosis is a condition that is common with aging and especially in post-menopausal women. The etiology has often been ascribed to abnormalities in calcium metabolism. However many patients with osteopenia/osteoporosis have in common pain and inflammation and many inflammatory pain syndromes have osteopenia/osteoporosis as an accompanying featureccxii. A notable example is the osteoporosis that is often present in Complex Regional Pain Syndrome/Reflex sympathetic dystrophy (CRPS-1/RSD)ccxiii. Interleukin-6 mediated inflammation has been shown to contribute to the process of bone remodeling. This it does by stimulating osteoclastogenesis and osteoclast activityccxiv. Elevated levels of Interleukin-6 have been observed in conditions of rapid skeletal turnover and hypercalcemia as in Paget's disease and multiple myelomaccxv. In multiple myeloma, radiologic examinations reveals osteolytic lesion and the most common finding is diffuse osteopeniaccxvi. Adhesion of multiple myeloma cells to stromal cells triggers IL-6 secretion by the stromal cellsccxvii. This results in increased osteoclastic activity that in turn results in osteoporosis, painful osteolytic lesions and hypercalcemia characteristic of multiple myelomaccxviii. In their youth, women are protected from osteoporosis because of the presence of sufficient levels of estrogen. Estrogen blocks the osteoblast's synthesis of Interleukin 6. Estrogen may also antagonize the interleukin 6 receptors. Decline in estrogen production is often associated with osteopenia/osteoporosis in postmenopausal women. Estrogen's ability to repress IL-6 expression was first recognized in human endometrial stromal cellsccxix. Additional clues came from the observations that menopause or ovariectomy resulted in increased IL-6 serum levelsccxx, increased IL-6 mRNA levels in bone cellsccxxi, and increased IL-6 secretion by mononuclear cellsccxxii ccxxiii ccxxiv. Further evidence for estrogen's ability to repress IL-6 expression is derived from studies, which demonstrated that estradiol inhibits bone marrow stromal cell and osteoblastic cell IL-6 protein and mRNA production in vitroccxxv and that estradiol was as effective as neutralizing antibody to IL-6 in suppressing osteoclast development in murine bone cell culturesccxxvi or in ovariectomized miceccxxvii.

OSTEOPOROSIS AND BISPHOSPHONATES Bisphosphonates are inorganic chemical compounds that bind to hydroxyapatite in bone and prevent osteoclastic absorption of bone. Nitrogen-containing bisphosphonates (N-BPs) are potent inhibitors of bone resorption widely used in the treatment of osteoporosis and other bone degrading disorders including Paget's disease of bone, hypercalcemia associated with malignancy, metastatic bone diseases, such as breast cancer, multiple myeloma, and arthritisccxxviii ccxxix. At the tissue level, N-BPs reduce bone turnover and increase bone mass and mineralization. This is measured clinically as an increase in bone mineral density and bone strength and a decrease in fracture risk. N-BPs localize preferentially at sites of bone resorption, where mineral is exposed, are taken up by ostoclasts and inhibit osteoclastic activity. At the molecular level, N-BPs inhibit an enzyme in the cholesterol synthesis pathway, farnesyl diphosphate synthase. As a result, there is a reduction in the lipid geranylgeranyl diphosphate, which prenylates GTPases required for cytoskeletal organization and vesicular traffic in the osteoclast, leading to osteoclast inactivationccxxx ccxxxi.

OSTEOPOROSIS AND STATINS 3-hydroxy-3-methylglutaryl coenzyme A reductase inhibitors (statins) have been shown to stimulate bone formation in laboratory studies, both in vitro and in vivo. Statin use in most, but not all observational studies is associated with a reduced risk of fracture, particularly hip fracture, even after adjustment for the confounding effects of age, weight and other medication use. This beneficial effect has not been observed in clinical trials designed to assess cardiovascular endpointsccxxxii. Men using statin drugs are more likely to have a greater BMD of the spine (p<0.005), and men who receive statin drugs for more than 2 yr are approximately half as likely to develop osteoporosis. A similar effect is observed in women taking statins for any length of timeccxxxiii. Statin use in women is associated with a 3% greater adjusted BMD at the femoral neck, and BMD tends to be greater at the spine and whole bodyccxxxiv. Nitrogen-containing bisphosphonate drugs inhibit the mevalonate pathway, preventing the production of isoprenoids, which consequently results in the inhibition of osteoclast formation and osteoclast function. Statins decrease the hepatic biosynthesis of cholesterol by blocking the mevalonate pathway, and can affect bone metabolism in vivo through effects on osteoclastic bone resorption. The ability of statin compounds to inhibit bone resorption is directly related to HMG-CoA reductase activityccxxxv.

OSTEOPOROSIS, PLANT POLYPHENOLS, AND FATTY ACIDS Dietary supplementation with soybean isoflavone can prevent postmenopausal bone loss. In one study, postmenopausal women (n=19), mean age 70.6+/−6.3 years and mean time since menopause 19.1+/−5.5 years, were given isoflavone supplements for 6 months. There was a 37% decrease in urinary concentrations of type 1 collagen alpha1-chain helical peptide (HP), a marker of bone resorption, during the isoflavone supplementation compared with baseline (p<0.05) and a significant difference in mean (SE) HP excretion levels when isoflavone was compared with placebo (43.4+/−5.2 vs. 56.3+/−7.2 microg/mmol creatinine [cr], p<0.05). With isoflavone supplementation, mean spine BMD at L2 and L3 was significantly greater when treatment was compared with control, with a difference between means of 0.03+/−0.04 g and 0.03+/−0.04 g (p<0.05), respectively. There were nonsignificant increases from baseline for total spine BMC (3.5%), total spine BMD (1%), total hip BMC (3.6%), and total hip BMD (1.3%) with the isoflavone treatmentccxxxvi. In another study, twenty-four 12-week-old Sprague-Dawley rats were divided randomly into 4 groups and given controlled diets for 16 weeks. The treatment groups were as followed: sham operated, ovariectomized (OVX) control, OVX+isoflavone extract (6.25 g/kg), and OVX+17beta-estradiol (4 mg/kg). OVX treatments reduced femoral and fourth lumbar vertebral bone density and mineral content (p<0.01), decreased uterine weight (p<0.01), accelerated body weight increases (p<0.05), and increased the activities (p<0.01) of both serum alkaline phosphatase (ALP) and tartrate-resistant acid phosphatase (TRAP). Supplementation with isoflavone prevented the losses of bone density and mineral content caused by OVX (p<0.01). Although both isoflavone and 17beta-estradiol exhibited similar bone-sparing ability on the OVX-induced bone loss, the effect of isoflavone was not the same as that of 17beta-estradiol on the serum ALP and TRAP, body weight increase, and uterine weight change. The authors concluded that dietary supplementation with soybean isoflavone can prevent postmenopausal bone loss via a different mechanism from estrogen in OVX ratsccxxxvii. Data from a randomized, double-blind, placebo-controlled, yearlong clinical trial has also suggested that supplementation with the dietary phytoestrogen genistein (54 mg/day) may be as effective as hormone replacement therapy in attenuating menopause-related bone lossccxxxviii

Several studies suggest that polyphenols might exert a protective effect against osteopenia. One experiment was conducted to observe the effects of rutin (quercetin-3-O-glucose rhamnose) on bone metabolism in ovariectomized (OVX) rats. Thirty 3-month-old Wistar rats were used. Twenty were OVX while the 10 controls were sham-operated (SH). Among the 20 OVX, for 90 days after surgery 10 were fed the same synthetic diet as the SH or OVX ones, but 0.25% rutin (OVX+R) was added. At necropsy, the decrease in uterine weight was not different in OVX and OVX+R rats. Ovariectomy also induced a significant decrease in both total and distal metaphyseal femoral mineral density, which was prevented by rutin consumption. Moreover, femoral failure load, which was not different in OVX and SH rats, was even higher in OVX+R rats than in OVX or SH rats. In the same way, on day 90, both urinary deoxypyridinoline (DPD) excretion (a marker for bone resorption) and calciuria were higher in OVX rats than in OVX+R or SH rats. Simultaneously, plasma osteocalcin (OC) concentration (a marker for osteoblastic activity) was higher in OVX+R rats than in SH rats. High-performance liquid chromatography (HPLC) profiles of plasma samples from OVX+R rats revealed that mean plasma concentration of active metabolites (quercetin and isorhamnetin) from rutin was 9.46+/−1 microM, whereas it was undetectable in SH and OVX rats. These results indicate that rutin (and/or its metabolites), which appeared devoid of any uterotrophic activity, inhibits ovariectomy-induced trabecular bone loss in rats, both by slowing down resorption and increasing osteoblastic activityccxxxix.

Beneficial effects of omega 3 fatty acids on bone mineral density have been reported in rats and humans. In one study, sham and ovariectomized (OVX) mice were fed diets containing either 5% corn oil (CO), rich in omega-6 fatty acids or 5% fish oil (FO), rich in omega-3 fatty acids. Bone mineral density was analyzed by DXA. The serum lipid profile was analyzed by gas chromatography. Receptor activator of NF-kappaB ligand (RANKL) expression and cytokine production in activated T-cells were analyzed by flow cytometry and ELISA, respectively. Significantly increased bone mineral density loss (20% in distal left femur and 22.6% in lumbar vertebrae) was observed in OVX mice fed CO, whereas FO-fed mice showed only 10% and no change, respectively. Bone mineral density loss was correlated with increased RANKL expression in activated CD4+ T-cells from CO-fed OVX mice, but there was no change in FO-fed mice. Selected n-3 fatty acids (docosahexaenoic acid [DHA] and eicosapentaenoic acid [EPA]) added in vitro caused a significant decrease in TRACP activity and TRACP+ multinuclear cell formation from BM cells compared with selected n-6 fatty acids (linoleic acid [LA] and arachidonic acid [AA]). DHA and EPA also inhibited BM macrophage NF-kappaB activation induced by RANKL in vitro. TNF-alpha, interleukin (IL)-2, and interferon (IFN)-gamma concentrations from both sham and OVX FO-fed mice were decreased in the culture medium of splenocytes, and interleukin-6 was decreased in sham-operated FO-fed miceccxl.

ARTHRITIS AND INTERLEUKIN-6 Interleukin-1 (IL-1), a cytokine produced by chondrocytes and other cells in the joint, plays an important role in cartilage degradation by stimulating the synthesis of degradative enzymes that inhibit the production of proteoglycans. Other cytokines that appear to act synergistically with IL-1 to promote matrix breakdown are tumor necrosis factor-alpha (TNF-alpha) and interleukin-6 (IL-6). During times of stress or inflammation IL-6 levels are increased. Inflammatory joint disease, particularly rheumatoid arthritisccxli, is associated with increased synovial fluid levels of IL-6ccxlii. Although Osteoarthritis has previously been considered a non-inflammatory form of arthritis, there are changes that occur within the joint that are associated with inflammation. Inflammation is aggravated by the introduction of bone and cartilage breakdown products into the synovial fluid. Cells in the synovium phagocytize these products, resulting in chronic, low-grade inflammation. Consequently, the synovial membrane becomes thickened. Inflammation of the synovial membrane may be absent in the earlier stages of Osteoarthritis; however, as the disease progresses, some degree of synovitis usually exists. Once mild synovial inflammation is established, the synovium becomes a source of cartilage-degrading enzymes (e.g., MMPs) and cytokines, including IL-1, IL-6, and TNF-alpha. These substances diffuse through the synovial fluid and cause further degradation of articular cartilage. IL-1 and TNF-alpha stimulate the chondrocytes to produce more degrading enzymes, and the process continues in a vicious cycle. IL-1, IL-6, and TNF-alpha are believed to be the main cytokines linked to the disease process.

ARTHRITIS AND BISPHOSPHONATES Pamidronate has resulted in pain reduction in patients with osteoarthritis (with and without osteoporosis) in our clinic, via its anti-inflammatory properties resulting in a subsequent reduction of bone resorption and inflammatory bone painccxliii. The quick onset of pain relief observed in our patients can only be attributed to its anti-interleukin-6 effect. Several literature abound documenting the anti-interleukin-6 effect of bisphosphonates. Bisphosphonates inhibit the production of pro-inflammatory cytokine interleukin-6 in tumoral cell lines of human osteoblastic phenotype (MG63 and SaOs cells), and in peripheral blood mononuclear cells (PBMC)ccxliv. Pamidronate infusion has significantly decreased the mean serum levels of Interleukin-6 in patients with advanced solid tumors and bone metastasesccxlv. Pamidronate and other bisphosphonates inhibit the production by osteoblasts of the inflammatory cytokine interleukin-6, a growth factor essential to myeloma cellsccxlvi. In patients with Paget's disease of bone, bisphosphonate therapy is associated with a significant reduction of Interleukin-6 soluble receptor (sIL-6R) serum levelsccxlvii. Bisphosphonates also inhibit IL-1 and TNF-alpha stimulated IL-6 release in cultures of human osteoblastic osteosarcoma cellsccxlviii. Osteoblasts exposed to small amounts of bisphosphonate elaborate a soluble inhibitor, which interferes with osteoclast formation and developmentccxlix. Furthermore, bisphosphonates prevent apoptosis of murine osteocytic ML0-Y4 cells, whether it is induced by etoposide, TNF-alpha, or the glucocorticoid dexamethasoneccl. In a recent study appearing in the journal, Clinical & Experimental Rheumatology Masuda-Aiba et al observed that a new third-generation bisphosphonate, YM529, represents a candidate treatment for arthritisccli. The authors report that prophylactic or therapeutic treatment of animals with experimental arthritis with YM529 suppressed the severity of disease and suggest that YM529 may act on arthritic joints locally to prevent inflammation. These data are consistent with previous clinical studies investigating the efficacy of other bisphosphonates in patients with rheumatoid arthritis. The authors concluded that although further experiments are necessary to elucidate the underlying mechanisms, YM529 deserves consideration as a treatment for this disease.

ARTHRITIS AND STATINS MMP-9 or Gelatinase B, a member of the matrix metalloproteinase family (MMPs), plays important roles in physiological events such as tissue remodeling and in pathological processes that lead to destructive bone diseases, including osteoarthritis and periodontitis. In addition to its effect on the increase of total bone mass, statin (an HMG-CoA reductase inhibitor) suppresses the expression of MMPs. In this study, the researchers proposed that simvastatin reduces MMP-9 expression in osteoblasts and HT1080 fibrosarcoma cell line. Gelatin zymography, Western blot analysis and reverse transcriptase-PCR were used to investigate the effects of simvastatin on MMP-9 in primary calvaria cells, U2-OS osteosarcoma cells, and HT1080 fibrosarcoma cells. The results from gelatin zymography and Western blot analysis revealed that simvastatin suppressed MMP-9 activity in these cells in concentration- and time-dependent manners. The effective concentrations of simvastatin were 100-500 nM, 5-15 microM, and 2.5-10 microM in primary calvaria, U2-OS, and HT1080 cells, respectively. The authors concluded that collectively, these results suggest that simvastatin is a potent drug for inhibition of MMP-9 expression in osteoblastic cells and HT1080 fibrosarcoma cellscclii. In another study, the researchers postulated that 3-Hydroxy-3-methylglutaryl-CoA reductase inhibitors (statins) exert favorable effects on lipoprotein metabolism, but may also possess anti-inflammatory properties. The authors explored the activities of simvastatin, a lipophilic statin, in a Th1-driven model of murine inflammatory arthritis. They reported in this study that simvastatin markedly inhibited not only developing but also clinically evident collagen-induced arthritis in doses that were unable to significantly alter cholesterol concentrations in vivo. Ex vivo analysis demonstrated significant suppression of collagen-specific Th1 humoral and cellular immune responses. Moreover, simvastatin reduced anti-CD3/anti-CD28 proliferation and IFN-gamma release from mononuclear cells derived from peripheral blood and synovial fluid. Proinflammatory cytokine production in vitro by T cell contact-activated macrophages was suppressed by simvastatin, suggesting that such observations have direct clinical relevance. The authors concluded that these data clearly illustrate the therapeutic potential of statin-sensitive pathways in inflammatory arthritisccliii. In one study, the authors set out to clarify whether the inhibition of sterol or nonsterol derivatives arising from mevalonate biotransformation plays a major role in the in vivo anti-inflammatory action of statinsccliv. Hepatic synthesis of all these derivatives was inhibited in mice by administered statins, whereas squalestatin inhibited only sterol derivatives. Using a short-term treatment schedule, the authors found that statins reduced the hepatic activity of 3-hydroxy-3-methylglutaryl coenzyme A reductase without affecting blood cholesterol. This treatment inhibited lipopolysaccharide- and carrageenan-induced pouch leukocyte recruitment and the exudate production of interleukin-6, monocyte chemotactic protein-1, and RANTES. Coadministration of mevalonate reversed the effect of statin on leukocyte recruitment. The inhibition of sterol synthesis by squalestatin did not have any anti-inflammatory effect, indicating that the biosynthesis of nonsterol compounds arising from mevalonate is crucial for the in vivo regulation of cytokine and chemokine production by statins. The authors concluded that inhibition by statins may account for the reported anti-inflammatory effects of these drugs and may provide a biochemical basis for the recently reported effects of statins in the prevention of cardiovascular disease and mortality.

ARTHRITIS, PLANT POLYPHENOLS, AND FATTY ACIDS One study investigated the impact of the isoflavone genistein on in vivo cell-mediated responses and collagen induced arthritis (CIA) in mice. Delayed type hypersensitivity reaction (DTH) to oxazolone and the inflammatory response to olive oil were measured in mice treated with genistein. In addition, the impact of genistein treatment on disease progression and outcome of collagen induced arthritis (CIA) was examined. The DTH reaction to oxazolone and the granulocyte-mediated response were significantly suppressed in genistein-treated as compared to control mice. Also, genistein treatment led to decreased levels of oxazolone-specific antibodies. Histologically, mice exposed to genistein and immunized with collagen II displayed somewhat lower degree of inflammation and joint destruction. In addition, serum levels of autoantibodies to collagen II were significantly lower following genistein-treatment in immunized mice. The authors concluded that genistein exerts evident anti-inflammatory properties affecting granulocytes, monocytes, and lymphocytescclv. Ipriflavone (7-isopropoxyisoflavone) is a synthetic derivative of naturally occuring isoflavones, flavonoid compounds found in soybeans and other plants. In one study, ipriflavone (TC-80) was given orally in a dose of 100 mg/kg/day for 3 weeks to rats with adjuvant arthritic chronic pain. Analgesic effects were observed 2 weeks after the start of administration in males and in ovariectomized estrone-supplemented females; the effect seen in the females was statistically significant. Changes in the bones of the hind paws were examined radiologically, and synovitis, periosteal new bone formation and bone destruction were examined histopathologically in the females. These variables were improved by treatment with TC-80 for 3 weekscclvi. One study examined the effect of a virgin olive oil enriched diet in acute and chronic inflammation models in rats and determined the effect of supplementing this oil with a higher content of its polyphenolic fraction. The response was compared to oils rich in monounsaturated fatty acids (high oleic sunflower oil and palm olein) and rich in polyunsaturated fatty acids (fish oil). Groups of 6-8 male Wistar rats were fed on six purified diets differing in type of oil: 2% corn oil (basal diet, BD), 15% high oleic sunflower oil (HOSO), 15% virgin olive oil (VOO), 15% virgin olive oil supplemented with 600 p.p.m. polyphenols from this oil (PSVOO), 15% palm olein (POL), and 15% fish oil (FO). Rats were fed for 8 weeks with BD, HOSO, VOO, PSVOO, POL and FO diets before injecting carrageenan. Rats were fed for 3 weeks with BD, PSVOO and FO diets before induction of adjuvant arthritis. Dietary treatment with or without indomethacin continued during 3 weeks. The data were evaluated using an analysis of variance (ANOVA) followed by the least-significant differences. In carrageenan oedema test, the inflammation indices of animals fed on a diet rich in olive oil (VOO) were lower compared to animals fed with oils high in oleic acid (HOSO, POL) and polyunsaturated fatty acids (FO), and markedly diminished in the group fed on PSVOO. In established adjuvant arthritis, the PSVOO diet was even more effective than FO diet in the prevention of inflammation. Both groups of animals showed an increase in weight during the latter days of the experiment compared to the BD. Indomethacin administered to every diet group, exerted a strong inhibitory effect on the inflammatory process throughout which was augmented by the PSVOO and FO diets. This study demonstrates that virgin olive oil with a higher content of polyphenolic compounds, similar to that of extra virgin olive oil, shows protective effects in both models of inflammation and improves the disease associated loss of weight. This supplementation also augmented the effects of anti-inflammatory drug therapycclvii. In another study, a polyphenolic fraction isolated from green tea (green tea polyphenols, GTPs) was shown to possess anti-inflammatory and anticarcinogenic properties in experimental animals. The study determined the effect of oral consumption of GTP on collagen-induced arthritis in mice. In three independent experiments, mice given GTP in water exhibited significantly reduced incidence of arthritis (33% to 50%) as compared with mice not given GTP in water (84% to 100%). The arthritis index also was significantly lower in GTP-fed animals. Western blot analysis showed a marked reduction in the expression of inflammatory mediators such as cyclooxygenase 2, IFN-gamma, and tumor necrosis factor alpha in arthritic joints of GTP-fed mice. Histologic and immunohistochemical analysis of the arthritic joints in GTP-fed mice demonstrated only marginal joint infiltration by IFN-gamma and tumor necrosis factor alpha-producing cells as opposed to massive cellular infiltration and fully developed pannus in arthritic joints of non-GTP-fed mice. The neutral endopeptidase activity was approximately 7-fold higher in arthritic joints of non-GTP-fed mice in comparison to nonarthritic joints of unimmunized mice whereas it was only 2-fold higher in the arthritic joints of GTP-fed mice. Additionally, total IgG and type II collagen-specific IgG levels were lower in serum and arthritic joints of GTP-fed mice. In conclusion, the authors suggest that a polyphenolic fraction from green tea may be useful in the prevention of onset and severity of arthritiscclviii.

Fish oils are a rich source of omega-3 long chain polyunsaturated fatty acids (n-3 LC PUFA). The specific fatty acids, eicosapentaenoic acid and docosahexaenoic acid, are homologues of the n-6 fatty acid, arachidonic acid (AA). This chemistry provides for antagonism by n-3 LC PUFA of AA metabolism to pro-inflammatory and pro-thrombotic n-6 eicosanoids, as well as production of less active n-3 eicosanoids. In addition, n-3 LC PUFA can suppress production of pro-inflammatory cytokines and cartilage degradative enzymescclix. Eicosanoids derived from the n-6 fatty acid, arachidonic acid, and the cytokines interleukin-1beta and tumor necrosis factor-alpha are involved in the signs and symptoms of inflammatory joint disease, as well as the cartilage degradation seen in established rheumatoid arthritis (RA). Then n-3 fatty acids in fish and fish oil can inhibit production of both eicosanoid and cytokine inflammatory mediators and therefore, have the potential to modify RA pathology. Epidemiological studies suggest that fish intake may be preventive for RA and double-blind placebo-controlled studies demonstrate that dietary fish oil can alleviate the signs and symptoms of RAcclx. 12 randomized, placebo-controlled double-blind studies, demonstrate a moderate but consistent improvement of clinical findings and laboratory parameters in patients with Rheumatoid Arthritis (RA). A dose-response relationship was established up to a daily dose of 2.6 gram fish oil, equivalent to about 1.6 gram EPA. In these experiments EPA was the omega-3 fatty acid responsible for improvement, with distinct effects on inhibition of cytokines formation (IL-1 to IL-6, IL-8, TFN-alpha, GM-CSF), decreased induction of proinflammatory adhesion molecules (selectines, intercellular adhesions molecule-1 (ICAM-1)), and degrading enzymes (e.g. phospholipase A2, cyclooxygenase-2, inducible NO-synthetase)cclxi.

DEMENTIA, ALZHEIMER'S DISEASE AND INTERLEUKIN 6 Vascular (formerly Arteriosclerotic) Dementia (MID, Multi-infarct dementia) is characterized by a history of transient ischemic attacks with brief impairment of consciousness, fleeting pareses, or visual loss. The dementia may also follow a succession of acute cerebrovascular accidents or, less commonly, a single major stroke. Some impairment of memory and thinking then becomes apparent. Onset, which is usually in later life, can be abrupt, following one particular ischemic episode, or there may be more gradual emergence. The dementia is usually the result of infarction of the brain due to vascular diseases, including hypertensive cerebrovascular disease. The infarcts are usually small but cumulative in their effect. Vascular dementia can occur with other types of dementia such as Alzheimer's disease. Compared with Alzheimer's disease, vascular dementia can affect distinct parts of the brain and particular abilities may remain relatively unaffected. Alzheimer's disease affects the entire brain. Symptoms of vascular dementia remain steady for a while and then suddenly decline. In Alzheimer's disease the decline is more constant.

Alzheimer's disease (AD), the most common form of dementia, is a progressive, degenerative disorder of the central nervous system. Interleukin 6 mediated inflammation play a role in several age-related diseases, including Alzheimer's disease. The Health, Aging and Body Composition Studycclxii enrolled 3,031 black and white men and women, with an average age of 74. The researchers took blood levels of interleukin-6 (IL-6), C-reactive protein and tumor necrosis factor and then repeated the tests two years later. A battery of mental tests was also given to evaluate concentration, memory, language and other measures of cognitive functioning, both at the start and two years later. After adjusting for age and other factors, they found that those who had the highest levels of inflammation—whose blood levels of IL-6 and C-reactive protein were in the highest one-third—had more cognitive decline compared to those whose blood levels of those substances were in the lower third. If their IL-6 result was high, they were 34 percent more likely to have cognitive decline than those whose scores on the tests were in the lower third. If their C-reactive protein levels were in the top third, they were 41 percent more likely to have cognitive decline than those in the lower third. Although those who suffered cognitive decline also had higher levels of tumor necrosis factor, the differences weren't statistically significant. The study found no relationship between the use of non-steroidal anti-inflammatory drugs (NSAIDs) and inflammation levels. This is not surprising as NSAIDs inhibit cyclooxygenase and affect prostaglandin synthesis but have no effect on IL-6 inflammation.

DEMENTIA, ALZHEIMER'S DISEASE AND STATINS Increased circulating cholesterol has been long linked to an increased risk of coronary artery disease (CAD), and is now linked to an increased risk of developing Alzheimer s disease (AD). The neuropathologic link between CAD and AD manifests as increased incidence of cerebral senile plaques in both disorders. In one study, the researchers showed that AD-like neuropathology occurred in the brains of cholesterol-fed rabbits; including increased beta-amyloid (A beta)cclxiii. The major hallmarks of AD include selective neuronal cell death and the presence of amyloid deposits and neurofibrillary tangles. Apolipoprotein E (ApoE) has also been shown to co-localize with these neuropathological lesions. Putative pathological functions or “risk-factor activities” of ApoE-epsilon4 include its role in promoting amyloid accumulation, neurotoxicity, oxidative stress and neuro fibrillary tangles. ApoE has been shown essential for amyloid beta-peptide fibrillogenesis and deposition, a defining pathological feature of this disease. The human ApoE gene has three alleles (epsilon2, epsilon3, epsilon4)-all products of the same gene. The epsilon3-allele accounts for the majority of the ApoE gene pool (approximately 70-80%), the epsilon-4-allele accounts for 10-15% and the epsilon2 allele for 5-10%. Inheritance of the epsilon-4-allele strongly increases the risk for developing AD at an earlier age. ApoE mRNA is most abundant in the liver followed by the brain, where it is synthesized and secreted primarily by astrocytes. ApoE protein and mRNA are further detected in cortical and hippocampal neurons in humans. ApoE gene expression is induced by brain injury in some neurons and upregulated in astrocytes during aging. In AD, an increased ApoE mRNA was reported in the hippocampus. The risk for AD has been reported to correlate with transcriptional activity of the ApoE gene. Binding sites for putative transcriptional factors (TF), such as AP-1, AP-2 and NF-kappaB, are present in the ApoE promoter. The promoter also contains sites for the inflammatory response transcription factors IL-6 RE-BP, MED 1, STAT1 and STAT2cclxiv.

Because astrocytes and microglia represent the major source of extracellular apoe in brain, one study investigated apoe secretion by glia. The authors determined that protein prenylation is required for apoe release from a continuous microglial cell line, primary mixed glia, and from organotypic hippocampal cultures. Using selective protein prenylation inhibitors, apoE secretion was found to require protein geranylgeranylation. This prenylation involved a protein critical to apoE secretion, not apoE proper. ApoE secretion could also be suppressed by inhibiting synthesis of mevalonate, the precursor to both types of protein prenylation, using hydroxyl-3-methylglutaryl coenzyme A reductase inhibitors (statins). The authors stated that recent reports have described the beneficial effects of statins on the risk of dementia. The authors further stated that their finding that protein geranylgeranylation is required for apoE secretion in the brain parenchyma provides another contributing mechanism to explain the effective properties of statins against the development of dementia. They concluded that in this model, statin-mediated inhibition of mevalonate synthesis, an essential reaction in forming geranylgeranyl lipid, would lower extracellular levels of parenchymal apoe. Because apoe has been found necessary for plaque development in transgenic models of Alzheimer's disease, suppressing apoe secretion by statins could reduce plaques and, in turn, improve cognitive functioncclxv.

Statins have been reported to mediate changes in neuronal survival and cytoskeleton, including the microtubule-associated protein tau, a major constituent of the tangles. In one study to determine the effect of statin on the cytoskeleton, the authors challenged rat primary neuron cultures by lovastatin and determined the metabolite that is critical for structural integrity and survival of neurons. During the blockade of 3-hydroxy-3-methylglutaryl-coenzyme A reductase, the neuritic plaque was affected and eventually was completely destroyed. This process was not part of the execution phase of apoptosis and was marked by alterations in the microfilament and microtubule system. The distribution and phosphorylation of protein tau changed. Immunoblot analysis and indirect immunofluorescence revealed a transient increase in tau phosphorylation, which ceased during the execution of apoptosis. The researchers determined that all of these effects could be linked to the lack of the geranylgeranylpyrophosphate intermediate. Inhibition of the geranylgeranylation of Rho family GTPases (geranylgeranyl-transferase I) evoked similar changes in neurons. The researchers stated that these data and their findings that statin treatment reduced the membrane-bound fraction of RhoA-GTPase in neurons suggest that reduced levels of functional small G proteins are responsible for the observed effects. They concluded that their data demonstrates that lovastatin concentrations that are able to suppress not only cholesterol but also geranylgeranylpyrophosphate formation may evoke phosphorylation of tau reminiscent of preclinical early stages of Alzheimer's disease and, when prolonged, apoptosiscclxvi.

An observational study of 1037 postmenopausal women with coronary heart disease enrolled in the Heart and Estrogen/progestin Replacement Study (participants at 10 of 20 centers), was undertaken to determine whether serum lipoprotein levels, the 4-year change in serum lipoprotein levels, and the use of statin drugs are associated with cognition in older women without dementia. The Modified Mini-Mental State Examination was administered at the end of the study after 4 years of follow-up. Women whose score was less than 84 points (>1.5 SDs below the mean) were classified as having cognitive impairment. Lipoprotein levels (total, high-density lipoprotein, and low-density lipoprotein [LDL] cholesterol and triglycerides) were measured at baseline and at the end of the study; statin use was documented at each visit. Compared with women in the lower quartiles, women in the highest LDL cholesterol quartile at cognitive testing had worse mean plus minus SD Modified Mini-Mental State Examination scores (93.7 plus minus 6.0 vs 91.9 plus minus 7.6; P=0.002) and an increased likelihood of cognitive impairment (adjusted odds ratio, 1.76; 95% confidence interval, 1.04-2.97). A reduction in the LDL cholesterol level during the 4 years tended to be associated with lower odds of impairment (adjusted odds ratio, 0.61; 95% confidence interval, 0.36-1.03) compared with women whose levels increased. Higher total and LDL cholesterol levels, corrected for lipoprotein(a) levels, were also associated with a worse Modified Mini-Mental State Examination score and a higher likelihood of impairment, whereas high-density lipoprotein cholesterol and triglyceride levels were not associated with cognition. Compared with nonusers, statin users had higher mean plus minus SD Modified Mini-Mental State Examination scores (92.7 plus minus 7.1 vs 93.7 plus minus 6.1; P=0.02) and a trend for a lower likelihood of cognitive impairment (odds ratio, 0.67; 95% confidence interval, 0.42-1.05), findings that seemed to be independent of lipid levels. The authors concluded that high LDL and total cholesterol levels are associated with cognitive impairment, and lowering these lipoprotein levels may be a strategy for preventing impairmentcclxvii. Another study examined the association between the use of lipid-lowering agents (LLAs) and dementia, adjusting for other markers of health, and investigated factors associated with LLA use. The authors performed a cohort study of LLA use and a case-control study of dementia in relation to LLA use, in a secondary analysis of the Canadian Study of Health and Aging(a nationally representative population-based survey of Canadians 65 years and older). To examine features associated with statin use, the authors evaluated data on 2305 people for whom health information, drug use, and cognitive status were known. To examine the relationship between LLA use and dementia, the authors selected incident cases of dementia (n=492, of whom 326 had Alzheimer disease) that occurred between the first and second waves of the study. Control subjects were 823 persons examined during the first and second phases of the Canadian Study of Health and Aging who had no cognitive impairment. Results from the study showed that use of LLAs was significantly (P<0.001) more common in younger (65-79 years) than in older (>or =80 years) people. It was not associated with other factors indicating a healthy lifestyle, but was associated with a history of smoking and hypertension. Use of statins and other LLAs reduced the risk of Alzheimer disease in subjects younger than 80 years, an effect that persisted after adjustment for sex, educational level, and self-rated health (odds ratio, 0.26; 95% confidence interval, 0.08-0.88). There was no significant effect in subjects 80 years and older. The researchers concluded that while the possibility of indication bias in the original observations cannot be excluded, it was not demonstrated in LLA use in this study. Lipid-lowering agent use was associated with a lower risk of dementia, and specifically of Alzheimer disease, in those younger than 80 yearscclxviii.

DEMENTIA, ALZHEIMER'S DISEASE AND BISPHOSPHONATES There is very little literature on the use of bisphosphonates in patients with dementia or Alzheimer's disease. In a clinical case report of primary hyperparathyroidism in an 89-year-old woman causing profound neuropsychiatric symptoms, the use of bisphosphonate therapy led to marked but temporary improvements in her mental statecclxix. Considering the role of Cholesterol in atherosclerosis, vascular dementia and Alzheimer's disease, bisphosphonates should play a future role in the prevention and treatment of dementia and Alzheimer's disease.

DEMENTIA, ALZHEIMER'S DISEASE, PLANT POLYPHENOLS, AND FATTY ACIDS Alzheimer's disease (AD) is a progressive neurodegenerative disorder pathologically characterized by deposition of beta-amyloid (Abeta) peptides as senile plaques in the brain. A hallmark of several human dementias including AD and fronto-temporal dementia with Parkinsonism on chromosome 17 (FTDP-17) is the hyperphosphorylation of the microtubule-associated protein tau. Preliminary experiments show that isoflavones delivered in a soy protein matrix attenuated selected AD-relevant tau phosphorylations in a primate model of menopausecclxx. In one study, regulation of amyloid precursor protein (APP) processing by protein kinase C (PKC) and phosphotyrosine pathways was investigated in cultured human astrocytes. Phorbol 12, 13-dibutyrate (PDBu), a PKC activator, increased secretion of APPalpha 2-3-fold over control values, and GF109203X, a PKC inhibitor, blocked this effect. Similarly, platelet derived growth factor (PDGF) increased the secreted form of APPalpha (sAPPalpha) level two-fold, and genistein, a tyrosine kinase inhibitor, blocked the stimulatory effect of PDGFcclxxi. Inhibition of the accumulation of amyloid beta-peptide (Abeta) and the formation of beta-amyloid fibrils (fAbeta) from Abeta, as well as the destabilization of preformed fAbeta in the CNS are attractive therapeutic targets for the treatment of Alzheimer's disease (AD). In another study, Nordihydroguaiaretic acid (NDGA) and wine-related polyphenols inhibit fAbeta formation from Abeta(1-40) and Abeta(1-42) as well as destabilizing preformed fAbeta(1-40) and fAbeta(1-42) dose-dependently in vitro. Using fluorescence spectroscopic analysis with thioflavin T and electron microscopic studies, the same researchers examined the effects of polymeric polyphenol, tannic acid (TA) on the formation, extension, and destabilization of fAbeta(1-40) and fAbeta(1-42) at pH 7.5 at 37 degrees C. in vitro. They then compared the anti-amyloidogenic activities of TA with myricetin, rifampicin, tetracycline, and NDGA. The study showed that TA dose-dependently inhibited fAbeta formation from Abeta(1-40) and Abeta(1-42), as well as their extension. Moreover, it dose-dependently destabilized preformed fAbetas. The effective concentrations (EC50) of TA for the formation, extension and destabilization of fAbetas were in the order of 0-0.1 microM. The authors concluded that TA could be a key molecule for the development of therapeutics for ADcclxxii. In a study, published in the Journal of Neuroscience, researchers studied the effects of treating mice genetically altered to develop Alzheimer's disease with high doses of epigallocatechin-3-gallate (EGCG), the main polyphenolic constituent of green tea. After several months of daily injections of EGCG, the results showed that EGCG reduced by as much as 54%. Abeta generation in both murine neuron-like cells (N2a) transfected with the human “Swedish” mutant amyloid precursor protein (APP) and in primary neurons derived from Swedish mutant APP-overexpressing mice (Tg APPsw line 2576). EGCG markedly promoted cleavage of the alpha-C-terminal fragment of APP and elevates the N-terminal APP cleavage product, soluble APP-alpha. These cleavage events were associated with elevated alpha-secretase activity and enhanced hydrolysis of tumor necrosis factor alpha-converting enzyme, a primary candidate alpha-secretase. As a validation of these findings in vivo, the study authors treated Tg APPsw transgenic mice overproducing Abeta with EGCG and found decreased beta-amyloid (Abeta) levels and plaques associated with promotion of the nonamyloidogenic alpha-secretase proteolytic pathway. The researchers concluded that these data raise the possibility that EGCG dietary supplementation may provide effective prophylaxis for ADcclxxiii. Low n-3 polyunsaturated fatty acid (PUFA) status may be associated with neuro-degenerative disorders, in particular Alzheimer's disease, which has been associated with poor dietary fish or n-3 PUFA intake, and low docosahexaenoic acid (DHA) status. One case-control study used an established biomarker of n-3 PUFA intake (serum cholesteryl ester-fatty acid composition) to determine n-3 PUFA status in patients with Alzheimer's disease, who were free-living in the community. All cases fulfilled the National Institute of Neurological and Communicative Disorders and Stroke and Alzheimer's Disease and Related Disorders Association criteria for Alzheimer's disease. Detailed neuropsychological testing and neuroimaging established the diagnosis in all cases. The subjects (119 females and twenty-nine males) aged 76.5 (SD 6.6) years had a clinical dementia rating (CDR) of 1 (SD 0.62) and a mini mental state examination (MMSE) score of 19.5 (SD 4.8). The control subjects (thirty-six females and nine males) aged 70 (SD 6.0) years were not cognitively impaired (defined as MMSE score <24): they had a mean MMSE score of 28.9 (SD 1.1). Serum cholesteryl ester-eicosapentaenoic acid and DHA levels were significantly lower (P<0.05 and P<0.001 respectively) in all MMSE score quartiles of patients with Alzheimer's disease compared with control values. Serum cholesteryl ester-DHA levels were progressively reduced with severity of clinical dementia. DHA levels did not differ in patients with Alzheimer's disease across age quartiles: all were consistently lower than in control subjects. Step-wise multiple regression analysis showed that cholesteryl ester-DHA and total saturated fatty acid levels were the important determinants of MMSE score and CDRcclxxiv. A 6-month randomized, placebo-controlled pilot study of the ethyl-ester of eicosapentaenoic acid (ethyl-EPA) was carried out in seven in-patients with advanced (stage III) Huntington's disease (three on ethyl-EPA, four on placebo; no significant difference in age or sex between the groups). After 6 months all the patients treated with ethyl-EPA improved on the orofacial component of the Unified Huntington's Disease Rating Scale while all the patients on placebo deteriorated on this scale (p<0.03). Following subvoxel registration of follow-up 3D MRI brain scans with baseline scans, subtraction images showed that while the placebo was associated with progressive cerebral atrophy, the ethyl-EPA was associated with a reverse processcclxxv.

HYPERTENSION, ARRHYTHMIAS AND INTERLEUKIN 6 IL-6 is elevated in plasma of preeclamptic women, and twofold elevation of plasma IL-6 increases vascular resistance and arterial pressure in pregnant rats, suggesting a role of the cytokine in hypertension of pregnancy. In one study, the authors tested the hypothesis that IL-6 directly impairs endothelium-dependent relaxation and enhances vascular contraction in systemic vessels of pregnant ratscclxxvi. Active stress was measured in aortic strips isolated from virgin and late pregnant Sprague-Dawley rats and then nontreated or treated for 1 h with IL-6 (10 pg/ml to 10 ng/ml). In endothelium-intact vascular strips, phenylephrine (Phe, 10-5 M) caused an increase in active stress that was smaller in pregnant (4.2±0.3) than virgin rats (5.1±0.3×104 N/m2). IL-6 (1,000 pg/ml) caused enhancement of Phe contraction that was greater in pregnant (10.6±0.7) than virgin rats (7.5±0.4×104 N/m2). The authors concluded that IL-6 inhibits endothelium-dependent NO-cGMP-mediated relaxation and enhances contraction in systemic vessels of virgin and pregnant rats. The greater IL-6-induced inhibition of vascular relaxation and enhancement of contraction in systemic vessels of pregnant rats supports a direct role for IL-6 as one possible mediator of the increased vascular resistance associated with hypertension of pregnancy.

Subcutaneous administration of Recombinant IL-6 is associated with cardiac arrhythmias, hepatotoxicity and significant increases in C-reactive protein (CRP), fibrinogen, platelet counts, lymphocyte IL-2 receptor levelscclxxvii. Inflammatory complications of surgery include atrial arrhythmias after cardiac surgery and there may exist a genetic predisposition to develop postoperative complications. One study was conceived to verify if a potential genetic modulator of the systemic inflammatory reaction to cardiopulmonary bypass (the −174 G/C polymorphism of the promoter of the Interleukin-6 gene) has a role in the pathogenesis of postoperative atrial fibrillation (AF). In 110 primary isolated coronary artery bypass patients the −174G/C Interleukin-6 promoter gene variant was determined. Interleukin-6, fibrinogen and C-reactive protein plasma levels were determined preoperatively, 24, 48, and 72 hours after surgery and at discharge. Heart rate and rhythm were continuously monitored for the first 36 to 48 hours; daily 12-lead electrocardiograms were performed thereafter until discharge. GG, CT, and CC genotypes were found in 62, 38, and 10 patients, respectively. Multivariate analysis (which included genotype, age, sex, and classical risk factors for AF) identified the GG genotype as the only independent predictor of postoperative AF. The latter occurred in 33.9% of GG versus 10.4% of non-GG patients (hazard ratio 3.25, 95% CI 1.23 to 8.62). AF patients had higher blood levels of Interleukin-6 and fibrinogen after surgery (P<0.001 for difference between the area under the curve). The −174G/C Interleukin-6 promoter gene variant appears to modulate the inflammatory response to surgery and to influence the development of postoperative AF. These data suggest an inflammatory component of postoperative atrial arrhythmias and a genetic predisposition to this complicationcclxxviii.

HYPERTENSION, ARRHYTHMIAS AND STATINS Recent studies have shown that short-term use of statins can reduce blood pressure (BP) significantly. To determine the long-term effects of statins on BP and aortic stiffness, a single-blind randomized prospective study was performed on 85 hyperlipidemic hypertensive patients whose BP was insufficiently controlled by antihypertensive therapy. Every 3 months, aortic stiffness was assessed by measuring pulse wave velocity (PWV). Patients were randomly allocated to groups treated with pravastatin, simvastatin, fluvastatin, or a nonstatin antihyperlipidemic drug. No significant differences in patient characteristics, kinds of antihypertensive drugs, BP, ankle brachial index, PWV, or serum lipid, creatinine, or C-reactive protein levels were found between the four groups at the start of the study. During the 12-month treatment period, PWV did not change in the pravastatin group or nonstatin group, but it was transiently reduced in the simvastatin group and significantly decreased in the fluvastatin group, even though the doses of the statins used in this study were lower than the usually prescribed dose. All four antihyperlipidemic drugs significantly decreased serum cholesterol levels without affecting BP, ankle brachial index, or serum triglyceride levels. The C-reactive protein serum levels decreased significantly in the three statin groups but not in the nonstatin group. The authors concluded that these results suggest that long-term use of fluvastatin by hyperlipidemic hypertensive patients is associated with a significant reduction in aortic stiffness without any effect on BPcclxxix. Other studies have suggested that lipid-lowering strategies, and particularly statins, could influence blood pressure (BP) control. The aim of the one study was to evaluate the effect of different lipid-lowering strategies on BP control of subjects with hypercholesterolemia who were enrolled in the prospective, population-based, longitudinal Brisighella Heart Study. A total of 1356 subjects with total cholesterol levels >or=239 mg/dL were randomly treated for 5 years (1988-1993) with 1 of these lipid-lowering regimens: low-fat diet, cholestyramine, gemfibrozil, or simvastatin. Participants were divided at baseline into 4 quartiles according to systolic BP level and examined for the percent change in systolic and diastolic BP during the 5 years of treatment. In the study results, a significant decrease in BP was observed in the 2 upper quartiles of systolic BP (>or=140 mm Hg) and was greater in subjects treated with cholesterol-lowering drugs who also had a greater reduction in plasma levels of low-density lipoprotein cholesterol. The BP decrease was greater in patients treated with statin drugs and, among those treated with anti-hypertensive drugs, in subjects in the fourth quartile. The authors concluded that the use of lipid-lowering measures could significantly improve BP control in subjects with both hypercholesterolemia and hypertension. The authors further stated that reduction in BP seems to be enhanced in subjects treated with statinscclxxx.

The preoperative use of statins is associated with a protective effect against postoperative atrial fibrillation/flutter (AF) independent of CRP levels. A prospective study was done of 131 patients in a tertiary care center (mean [+/−SD] age, 73+/−6 years) who had undergone major lung or esophageal resection. High-sensitivity CRP and interleukin (IL)-6 levels were measured before surgery, on arrival at the postanesthesia care unit, and on the first morning after surgery. Continuous telemetry was used for 72 to 96 h to detect AF. AF occurred in 38 of 131 patients (29%) at a median time after surgery of 3 days. Although CRP and IL-6 levels increased significantly (p<0.001) in response to surgery, patients with or without AF did not differ in perioperative values. In a stepwise logistic regression, statin use was associated with a threefold decrease in the odds of developing AF (odds ratio [OR], 0.26; 95% confidence interval [CI], 0.08 to 0.82; p=0.022) and a greater PR interval (OR, 1.11 per 5-ms increments; 95% CI, 1.01 to 1.22; p=0.027) predicted an increase in the risk of AFcclxxxi.

HYPERTENSION, ARRHYTHMIAS AND BISPHOSPHONATES There is very little literature on the use of bisphosphonates in patients with hypertension or arrhythmias. Considering the role of Interleukin-6 in atherosclerosis, bisphosphonates should play a future role in the prevention and treatment of hypertension and certain types of arrhythmia.

HYPERTENSION, ARRHYTHMIAS, PLANT POLYPHENOLS, AND FATTY ACIDS Activation of tyrosine kinase appears to play an important role in pathogenesis of cardiovascular disease during chronic hypertension. One study tested the hypothesis that long-term treatment with an inhibitor of tyrosine kinase would have beneficial effects on hypertension-induced morphological and functional changes of the cerebral artery. Male spontaneously hypertensive rats (SHR; 4 months old) were fed normal rat chow, or that containing an inhibitor of tyrosine kinase, genistein (1 mg/kg chow). Normotensive Wistar-Kyoto (WKY) rats were also fed either of the chows. After feeding the rats for 2 months, the researchers measured wall thickness, diameter of the basilar artery and its dilator responses to acetylcholine (ACh); Y-26763, an opener of ATP-sensitive potassium channels; and Y-27632, an inhibitor of Rho-associated kinase. Genistein treatment reduced the wall thickness significantly in SHR. Vasodilator responses induced by ACh and Y-26763 were markedly attenuated in SHR compared to WKY rats, and treatment of SHR with genistein significantly improved the vasodilatation. Dilatation of the artery in response to Y-27632 was enhanced in SHR compared to WKY rats and treatment of SHR with genistein did not affect the enhanced vasodilator responses to Y-27632. The authors concluded that chronic treatment with genistein may be a novel approach to prevent cerebrovascular disorderscclxxxii. The possibility that the heightened cardiovascular risk associated with the menopause can be reduced by increasing dietary isoflavone intake was tested in 17 women by measuring arterial compliance, an index of the elasticity of large arteries such as the thoracic aorta. Compliance diminishes with age and menopause. An initial 3- to 4-week run-in period and a 5-week placebo period were followed by two 5-week periods of active treatment with 40 mg and then 80 mg isoflavones derived from red clover containing genistein, daidzein, biochanin, and formononetin in 14 and 13 women, respectively, with 3 others serving as placebo controls throughout. Arterial compliance, measured by ultrasound as a pressure (carotid artery) and volume (outflow into aorta) relationship, was determined after each period; plasma lipids were measured twice during each period. Urinary output of isoflavones was also determined. Arterial compliance rose by 23% relative to that during the placebo period with the 80-mg isoflavone dose and slightly less with the 40-mg dose (mean+/−SEM: placebo, 0.197+/−0.015; 40 mg, 0.237+/−0.007; 80 mg, 0.244+/−0.014). In the three women receiving continuous placebo, compliance was 0.16+/−0.022, similar to that during the run-in period for the remaining subjects (0.17+/−0.021) [corrected]. ANOVA showed a significant (P=<0.001) difference between treatments; by Bonferroni multiple comparisons and by paired t test, differences were significant between placebo and 40- and 80-mg isoflavone doses (by paired t test: P=0.039 for placebo vs. 40 mg; P=0.018 for placebo vs. 80 mg). Plasma lipids were not significantly affected. An important cardiovascular risk factor, arterial compliance, which diminishes with menopause, was significantly improved with red clover isoflavones. As diminished compliance leads to systolic hypertension and may increase left ventricular work, the study findings indicate a potential new therapeutic approach for improved cardiovascular function after menopausecclxxxiii. Epidemiologic studies indicate that tea consumption slightly reduces blood pressure. One study was conducted to determine whether black and green tea can lower blood pressure (BP) in stroke-prone spontaneously hypertensive rats (SHRSP). Male SHRSP (n=15) were allowed to recover for 2 wk after a transmitter for measuring BP was implanted in the peritoneal cavity. The rats were divided into three groups: the control group consumed tap water (30 mL/d); the black tea polyphenol group (BTP) consumed water containing 3.5 g/L thearubigins, 0.6 g/L theaflavins, 0.5 g/L flavonols and 0.4 g/L catechins; and the green tea polyphenol group (GTP) consumed water containing 3.5 g/L catechins, 0.5 g/L flavonols and 1 g/L polymetric flavonoids. The telemetry system was used to measure BP, which were recorded continuously every 5 min for 24 h. During the daytime, systolic and diastolic BP were significantly lower in the BTP and GTP groups than in the controls. The amounts of polyphenols used in this experiment correspond to those in approximately 1 L of tea. The study authors concluded that the regular consumption of black and green tea may also provide some protection against hypertension in humanscclxxxiv.

High doses of fish oil have been shown to reduce blood pressure in men with essential hypertension A population-based, randomized, 10-week dietary-supplementation trial was conducted in which the effects of 6 g per day of 85 percent eicosapentaenoic and docosahexaenoic acids were compared with those of 6 g per day of corn oil in 156 men and women with previously untreated stable, mild essential hypertension. The mean systolic blood pressure fell by 4.6 mm Hg (P=0.002), and diastolic pressure by 3.0 mm Hg (P=0.0002) in the group receiving fish oil; there was no significant change in the group receiving corn oil. The differences between the groups remained significant for both systolic (6.4 mm Hg; P=0.0025) and diastolic (2.8 mm Hg; P=0.029) pressure after control for anthropometric, lifestyle, and dietary variables. The decreases in blood pressure were larger as concentrations of plasma phospholipid n-3 fatty acids increased (P=0.027)cclxxxv. Studies have shown that docosahexaenoic acid (DHA) has an antihypertensive effect in spontaneously hypertensive rats (SHR). In one study investigated possible mechanisms for this effect. Vascular pathology and reactivity were determined in SHR treated with dietary DHA. SHR (7 weeks) were fed a purified diet with either a combination of corn/soybean oils or a DHA-enriched oil for 6 weeks. Histological evaluation of heart tissue, aorta, coronary, and renal arteries was performed. Vascular responses were determined in isolated aortic rings. Contractile responses to agonists, including norepinephrine (10(−9) to 10(−4) M), potassium chloride (5-55 mM), and angiotensin 11 (5×10(−7) M) were assessed. Vasorelaxant responses to acetylcholine (10(−9) to 10 (−4) M), sodium nitroprusside (10(−9) to 10(−6) M), papaverine (10(−5) to 10(−4) M), and methoxyverapamil (D600, 1-100 microM) were determined. DHA-fed SHR had significantly reduced blood pressure (P<0.001) and vascular wall thicknesses in the coronary, thoracic, and abdominal aorta compared with controls (P<0.05) Contractile responses to agonists mediated by receptor stimulation and potassium depolarization were not altered in DHA-fed SHR. Endothelial-dependent relaxations to acetylcholine were not altered which suggests endothelial-derived nitric oxide production/release is not affected by dietary DHA. Other mechanisms of vascular relaxation, including intracellular cyclic nucleotides, cGMP, and cAMP were not altered by dietary DHA because aortic relaxant responses to sodium nitroprusside and papaverine were similar in control and DHA-fed SHR. No significant differences were seen in relaxant responses to the calcium channel blocker, D600, or contractile responses to norepinephrine in the absence of extracellular calcium. These results suggest that dietary DHA does not affect mechanisms related to extracellular calcium channels or intracellular calcium mobilization. Moreover, the contractile and vasorelaxant responses are not differentially altered with dietary DHA in this in vivo SHR model. The findings demonstrate that dietary DHA reduces systolic blood pressure and vascular wall thickness in SHR. This may contribute to decrease arterial stiffness and pulse pressure, in addition to the antihypertensive properties of DHAcclxxxvi.

Epigallocatechin, a green tea polyphenol, attenuates myocardial ischemia reperfusion injury in rats.

In one study, Male Wistar rats were subjected to myocardial ischemia (30 min) and reperfusion (up to 2 h). Rats were treated with EGCG (10 mg/kg intravenously) or with vehicle at the end of the ischemia period followed by a continuous infusion (EGCG 10 mg/kg/h) during the reperfusion period. In vehicle-treated rats, extensive myocardial injury was associated with tissue neutrophil infiltration as evaluated by myeloperoxidase activity, and elevated levels of plasma creatine phosphokinase. Vehicle-treated rats also demonstrated increased plasma levels of interleukin-6. These events were associated with cytosol degradation of inhibitor kappaB-alpha, activation of IkappaB kinase, phosphorylation of c-Jun, and subsequent activation of nuclear factor-kappaB and activator protein-1 in the infarcted heart. In vivo treatment with EGCG reduced myocardial damage and myeloperoxidase activity. Plasma IL-6 and creatine phosphokinase levels were decreased after EGCG administration. This beneficial effect of EGCG was associated with reduction of nuclear factor-kB and activator protein-1 DNA bindingcclxxxvii.

Epidemiological studies and clinical trials report the beneficial effects of fish or fish oil consumption on cardiovascular disease outcomes including sudden death. One study performed a systematic review of the literature on controlled animal studies that assessed the effects of omega-3 fatty acids on selected arrhythmia outcomes. On the basis of predetermined criteria, 27 relevant animal studies were identified; 23 of these were feeding studies, and 4 were infusion studies. Across species, fish oil, eicosapentaenoic acid, and/or docosahexaenoic acid appear to have beneficial effects on ventricular tachycardia (VT) and fibrillation (VF) in ischemia-but not reperfusion-induced arrhythmia models; no effect on the incidence of death and infarct size; and inconsistent results with regard to arrhythmia score, VF threshold, ventricular premature beats or length of time in normal sinus rhythm, compared to omega-6, monounsaturated, or saturated fatty acids, and no treatment controls. In a meta-analysis of 13 studies using rat models, fish oil but not alpha-linolenic acid supplementation showed a significant protective effect for ischemia- and reperfusion-induced arrhythmias by reducing the incidence of VT and VF. The researchers concluded that it is not known whether omega-3 fatty-acid supplementation has antiarrhythmic effects in other disease settings not related to ischemiacclxxxviii. Another study examined associations of fish consumption with ischemic heart disease (1HD) risk among older adults or how different types of fish meals relate to IHD risk. In a population-based prospective cohort study, usual fish consumption was ascertained at baseline among 3910 adults aged >or=65 years and free of known cardiovascular disease in 1989 and 1990. Consumption of tuna and other broiled or baked fish correlated with plasma phospholipid long-chain n-3 fatty acids, whereas consumption of fried fish or fish sandwiches (fish burgers) did not. Over 9.3 years' mean follow-up, there were 247 IHD deaths (including 148 arrhythmic deaths) and 363 incident nonfatal myocardial infarctions (MIs). After adjustment for potential confounders, consumption of tuna or other broiled or baked fish was associated with lower risk of total IHD death (P for trend=0.001) and arrhythmic IHD death (P=0.001) but not nonfatal MI (P=0.44), with 49% lower risk of total IHD death and 58% lower risk of arrhythmic IHD death among persons consuming tuna/other fish 3 or more times per week compared with less than once per month. In similar analyses, fried fish/fish sandwich consumption was not associated with lower risk of total IHD death, arrhythmic IHD death, or nonfatal MI but rather with trends toward higher risk. Among adults aged >or=65 years, modest consumption of tuna or other broiled or baked fish, but not fried fish or fish sandwiches, is associated with lower risk of IHD death, especially arrhythmic IHD death. The researchers concluded that cardiac benefits of fish consumption may vary depending on the type of fish meal consumedcclxxxix.

CANCER AND INTERLEUKIN 6 Programmed cell death or apoptosis is a genetically coded cellular mechanism by which cells activate pathways that promote suicide. Apoptosis causes cells to shrink and be eliminated without the tissue trauma associated with inflammation that accompanies uncontrolled cell death (necrosis). Apoptosis can benefit the organism by eliminating defective cells and protecting from cancer. Apoptosis is defined by morphological characteristics, including cytoplasmic shrinkage, nuclear condensation, and DNA fragmentation. Apoptosis is vital at many stages of development in higher organisms and remains important for homeostasis throughout their lifetime. Signal transduction pathways influence and control apoptosis. Signaling pathways controlling apoptosis are implicated in the aging process and aging related diseases, including cancer and neurodegenerative diseases.

In apoptosis proteolytic enzymes (notably caspases—Cysteine Aspartase ProteASES) begin the process of orderly protein degradation that culminates in the production of small packages of cellular remnant. Apoptosis initiated by an extracellular signal (Fas receptor) activates caspase 8, whereas apoptosis due to intracellular damage or distress activates caspase 9. The oncogene protein p53 is a potent initiator of apoptosis, whereas the oncogene protein Bcl-2 is a potent inhibitor.

Mitogens are agents that trigger mitosis (cell division). Growth factors and stress are mitogens. Active cell proliferation (mitosis) is essential to growth & development in a young organism. However, in an older organism proliferation is often associated with inflammation and more easily leads to cancer. Mitogens generally act at cell surfaces, and cell signaling resulting from surface stimulation is by Mitogen Activated Protein Kinases (MAPKs). MAPK pathways are typically a series of kinases that activate other kinases. There are three families of MAPKs: (1) Extracellular signal-Regulated Kinases (ERKs), (2) c-Jun N-terminal Kinases (JNKs) and (3) the p38 family of kinases. The ERK family responds to growth factors, resulting in proliferation & differentiation, whereas the other two families respond to a variety of stresses or inflammatory cytokines that can lead either to apoptosis or to proliferation—depending on the tissue & stimulation. The most important inflammatory kinase is p38. Activator Protein-1 (AP-1) is a transcription factor activated by either ERK or JNK.

A crucial biochemical event required for most apoptotic responses is the activation of proteases of the caspase subfamily. A subset of signaling proteins is cleaved by caspases during apoptosis. One of these proteins is the MAPK1 kinase kinase MEKK1, which regulates the ERK and the JNK MAPK pathways, as well as the transcription factor NFκB and the p300 transcriptional co-activator. Expression of the kinase domain of MEKK1 into cells induces apoptosis in a manner that depends on a functional kinase activity. MEKK1 is necessary for apoptosis caused by detachment from the extracellular matrix (anoikis) in Madin-Darby canine kidney cells or in response to UV-C irradiation and several chemotherapeutic drugs. In these situations, MEKK1 is cleaved by caspases into a 91-kDa kinase-containing fragment that further stimulates the activation of caspases and, consequently, apoptosis. The kinase domain of MEKK1 may also favor apoptosis by inducing an increased expression of Fas and Fas ligand.ccxc

3-hydroxy-3-methylglutaryl coenzyme A (HMG-CoA) reductase activity is elevated in malignant cells. Increased synthesis of mevalonate and mevalonate-derived nonsterol isoprenoids supports increased cell proliferation through the activation of growth-regulatory proteins and oncoproteins, and by promoting DNA synthesis. Mevalonate has been shown to promote the growth of human breast cancer cells both in culture and as tumors grown in nude miceccxci. Dysregulation of the JAK-STAT pathway is frequently observed in many primary human tumors, reflecting the importance of this pathway in the maintenance of cellular integrityccxcii. Vascular endothelial growth factor (VEGF) upregulation is induced by many receptor and intracellular oncogenic proteins commonly activated in cancer. Two major transcription activators have been identified for VEGF promoter: hypoxia inducible factor-1 (HIF-1) and signal transducer and activator of transcription (STAT3). Both HIF-1 expression and STAT3 activity are upregulated in diverse cancers. STAT3 is required for both basal and growth signal-induced expression of HIF-1, and induction of VEGF by diverse oncogenic growth stimuli, including IL-6R, c-Src, Her2/Neu, is attenuated in cells without STAT3 signaling. Targeting STAT3 with a small-molecule inhibitor blocks HIF-1 and VEGF expression in vitro and inhibits tumor growth and angiogenesis in vivo. Furthermore, tumor cells' in vivo angiogenic capacity induced by IL-6R, which simultaneously activates Jak/STAT and PI3K/Akt pathways, is abrogated when STAT3 is inhibitedccxciii. Persistent activation of STAT3, is a common feature of prostate cancer. Activated STAT3 is found in the cancerous areas of pathology specimens obtained from prostatectomy but not in the normal marginsccxciv. IL-6 triggers proliferation of myeloma cell tumors via the Ras-mitogen-activated protein kinase (MAPK) cascade and is thought to promote tumor survival via signal transducer and activator of transcription (STAT) pathway-dependent regulation of Bcl-2 family antiapoptotic membersccxcv. IL-6 is elevated in malignant gliomas, and glioma cells respond to IL-6. Phosphorylation and nuclear translocation of the transcription factor signal transducer and activator of transcription (STAT3), is a prerequisite for IL-6 signaling, in human gliomas and experimental mouse tumorsccxcvi. IL-6, IL-6 receptor alpha (IL-6Ralpha), and gp130 are expressed in human esophageal carcinoma tissues. In one study, IL-6 protected an esophageal carcinoma cell line CE48T/VGH from apoptosis induced by staurosporine. IL-6 stimulation induced a rapid phosphorylation of gp130 and STAT3, and a dominant-negative STAT3 completely abolished the antiapoptotic effect. IL-6 also activated ERK 1/2 in CE48T/VGH cells. Inhibition of the ERK activation by PD98059 and transfection of a dominant-negative ERK2 completely blocked the protection of IL-6 against apoptosis. The authors concluded that STAT and MAP kinase pathways are responsible for the IL-6-delivered survival signal in human esophageal carcinoma cellsccxcvii. A high activity of STAT-3 has also been found in chemically-induced rat hepatocellular carcinomas (HCCs)ccxcviii.

The suppressor of cytokine signaling-1 (SOCS1) down-regulates Janus kinases/signal transducers and activators of transcription (JAK/STAT) pathway activity and inhibits the biological effects of cytokines. SOCS1 has been shown to have tumor-suppressor activity, and methylation of this gene, resulting in transcriptional silencing, has been found in 65% of hepatocellular carcinoma and more than half of patients with newly diagnosed acute myeloid leukemia (AML). SOCS1 has been suggested to play an important role in the development of these cancersccxcix. Aberrant SOCS-1 methylation has also been found in the IL-6-dependent multiple myeloma (MM) cell lines U266 and XG1, which correlated with transcriptional silencing. Using methylation-specific polymerase chain reaction (MSP), researchers found that SOCS-1 hypermethylated in 62.9% (23/35) of MM patient samples. Silencing of the SOCS-1 gene may impair negative regulation of the Jak/STAT pathway, thus supporting survival and expansion of MM cellsccc. Tumor progression is a complex process that depends on interactions between tumor and host cells. One aspect of the host response, the inflammatory response, is of particular interest because it includes the release of proinflammatory cytokines, some of which may promote tumor growth and hence influence survival. Interleukin-6 (IL-6) is a pleiotropic cytokine that regulates growth and differentiation of various types of malignant tumors. IL-6 is produced in response to a variety of stimuli, and is required for the development of T and B lymphocytes to effector cells. In certain neoplasias, such as multiple myeloma, IL-6 is both produced and required for survival by the cancer cell itself. In other neoplasias, IL-6 may come from tissue surrounding the tumour. IL-6 is a pathophysiological factor in several hyperproliferative diseases and the paraneoplastic syndromes that often accompany cancer, such as cachexia and osteoporosisccci IL-6 signals in target tissues through the receptor that is composed of the ligand-binding and signal-transducing subunits.

The nuclear transcription factors nuclear factor-kappaB (NF-kappaB) and signal transducer and activator of transcription 3 (STAT3) play a central role in chemoresistance, cell survival, and proliferation in patients with multiple myeloma (MM). One study investigated whether MM cells derived from patients express activated NF-kappaB and STAT3 and if their suppression induces apoptosis. The authors assayed CD138+ cells from the bone marrow of 22 MM patients and checked for the activated forms of NF-kappaB and STAT3 by immunocytochemistry. The researchers found that MM cells from all the patients expressed the activated forms of NF-kappaB and STAT3 but to a variable degree (NF-kappaB: low, 3 of 22; moderate, 5 of 22; or high, 14 of 22; STAT3: none, 1 of 22; low, 3 of 22; moderate, 5 of 22; or high, 14 of 22). Constitutive activation of NF-kappaB was in some cases also independently confirmed by electrophoretic mobility gel shift assay. In contrast to MM patients, activated forms of NF-kappaB and STAT3 were absent in cells from healthy individuals. Suppression of NF-kappaB and STAT3 activation in MM cells by ex vivo treatment with curcumin (diferuloylmethane) resulted in a decrease in adhesion to bone marrow stromal cells, cytokine secretion, and in the viability of cells. The authors concluded that fresh cells from MM patients express constitutively active NF-kappaB and STAT3, and suppression of these transcription factors inhibits the survival of the cellscccii. In another study, Curcumin down-regulated the expression of NF-kappaB-regulated gene products, including IkappaBalpha, Bcl-2, Bcl-x(L), cyclin D1, and interleukin-6. This led to the suppression of proliferation and arrest of human multiple myeloma (MM) cells at the G(1)/S phase of the cell cycleccciii. IL-6 is expressed in benign and malignant prostate tissue and the levels of the cytokine and its receptor increase during prostate carcinogenesis. Activation of signaling pathways of Janus kinase/signal transducers and activators of transcription factors, mitogen-activated protein kinase (MAPK), and phosphatidylinositol 3-kinase has been reported in various prostate cancer cell linesccciv. IL-6 levels in the serum of patients with hormone refractory and metastatic prostate cancer are significantly increased compared with those in patients with hormone sensitive and localized prostate cancercccv. In one study to evaluate how NF-kappaB signaling in tumor cells regulates processes associated with osteolytic bone tumor burden, the researchers stably infected the bone-seeking MDA-MB-231 breast cancer cell line with a dominant-negative mutant IkappaB that prevents phosphorylation of IkappaBalpha and associated nuclear translocation of NF-kappaB. Blockade of NF-kappaB signaling in MDA-MB-231 cells by the mutant IkappaB decreased in vitro cell proliferation, expression of the proinflammatory, bone-resorbing cytokine interleukin-6, and in vitro bone resorption by tumor/osteoclast cocultures while reciprocally up-regulating production of the proapoptotic enzyme caspase-3. Suppression of NF-kappaB transcription in these breast cancer cells also reduced incidence of in vivo tumor-mediated osteolysis after intratibial injection of tumor cells in female athymic nude mice. Immunohistochemistry showed that the cancerous lesions formed in bone by MDA-MB-231 cells express both interleukin-6 and the p65 subunit of NF-kappaB at the bone-tumor interface. The authors concluded that NF-kappaB signaling in breast cancer cells therefore promotes bone tumor burden and tumor-mediated osteolysis through combined control of tumor proliferation, cell survival, and bone resorptioncccvi. The pretreatment serum IL-6 level is a predictive factor of overall survival in metastatic malignant melanoma (MMM). In a study to establish the possible relationship between IL-6 level and overall survival in MMM, patients were divided into two groups according to a cut-off of 5 pg/ml, corresponding to the first quartile obtained by descriptive statistics of the pretreatment IL-6 level in all patients. Thirty-five patients were in the low IL-6 group and 76 patients were in the high IL-6 group. Based on this stratification, overall survival was shown to be affected by IL-6 serum level: it was higher (24.6 months) in the low IL-6 group when compared with the high IL-6 group (9.7 months) (P=0.0006)cccvii. Elevated IL-6 is associated with a poorer prognosis among ovarian cancer patients and has been implicated in the metastasis of ovarian cancercccviii. Gastric carcinoma occurs in response to chronic inflammation of gastric mucosa infected with Helicobacter pylori. One study measured tissue concentrations of the proinflammatory cytokines interleukin (IL)-1beta and IL-6 in gastric carcinoma and investigated the correlation between the levels of these cytokines and clinicopathological features. Biopsy specimens of tumors or adjacent normal mucosa were obtained from 42 Japanese patients with gastric carcinoma. Tissue levels of IL-1beta and IL-6 were measured by enzyme-linked immunosorbent assay. IL-1beta levels were significantly higher in the neoplasm than in the corresponding normal mucosa. The IL-6 levels in the neoplasm correlated significantly with the depth of invasion and lymphatic invasioncccix. High levels of IL-1beta and IL-6 were characteristic of non-scirrhous type gastric carcinoma. Interleukin-6 (IL-6) is produced at high levels by renal cell carcinoma cell lines. In one study, IL-6 and IL-6 receptor expression was investigated in 8 renal cell carcinoma (RCC) cell lines. The modulation of RCC cell line proliferation by an anti-IL-6 Ab, an IL-6 antisense oligonucleotide (ASON) directed against the second exon of IL-6 and cytokines inhibiting IL-6 production (IL-4 and IL-13) was investigated. All 8 RCC cell lines expressed IL-6 mRNA, produced IL-6 and expressed the soluble and membrane-bound gp130 chain of IL-6 receptor. The gp80 chain of IL-6 receptor was undetectable at the surface of the 8 RCC cell lines tested, while the soluble form of gp80 was detectable in the supernatant of one of these cell lines. The addition of a blocking IL-6 Ab did not inhibit the proliferation of any of the 8 RCC cell lines. In contrast, IL-6 ASON inhibited specifically IL-6 production and the proliferation of all RCC cell linescccx. In another study, administration of a novel peptide, S7, which selectively binds to IL-6 receptor (IL-6R) alpha chain (gp80) and broadly inhibits IL-6-mediated events prevents IL-6-mediated survival signaling and sensitizes cervical cancer cells to chemotherapeutic compounds in vitro. The in vitro analysis of antiangiogenic activity showed that S7 peptide substantially inhibits IL-6-induced vascular endothelial growth factor-A expression and angiogenesis in different cancer cell lines. Furthermore, S7 peptide was bioavailable in vivo, leading to a significant suppression of IL-6-induced vascular endothelial growth factor-mediated cervical tumor growth in severe combined immunodeficient micecccxi. Anti-IL-6 antibodies, as well as Anti-IL-6 receptor antibodies (e.g., Atlizumab: a humanized monoclonal anti-IL-6 receptor antibody; Biodrugs. 17(5):369-372, 2003, incorporated herein by reference) are particularly useful within the methods and compostions of the invention.

Anti-IL-6 receptor antibodies for use in the present invention can be obtained as polyclonal or monoclonal antibodies. Monoclonal antibodies of a mammalian origin include those produced by hybridomas and those produced by host cell which has been transformed with an expression vector containing genetically engineered antibody genes. Examples of such antibodies include PM-1 antibody (Hirata, et al., J. Immunology (1989) 143, 2900-2906), MR16-1 antibody (Tamura, T., et al., Proc. Natl. Acad. Sci. USA (1993) 90, 11924-11928), or AUK12-20 antibody, AUK64-7 antibody or AUK146-15 antibody (International Patent Publication WO 92-19759), and the like. Thus, in an embodiment of the invention, humanized anti-Interleukin-6 receptor monoclonal antibody is one of the methods of inhibition of Interleukin-6 mediated inflammation. These include humanized monoclonal antibody, PM-1 against human IL-6 receptor, humanized monoclonal antibody MR16-1 against human IL-6 receptor and humanized monoclonal antibody AUK146-15, AUK12-20, AUK64-7 against human IL-6 receptor.

The prevalence of depression among patients diagnosed with cancer is higher than among the general medical population and is associated with faster tumor progression and shortened survival time. Cancer-related depression often occurs in association with anorexia and cachexia, although until recently the relationship between these conditions has not been well understood. Cachexia is associated with poorer quality of life and survival outcomes and is the eventual cause of death in approximately 30% of all patients with cancer. Recent evidence has linked elevated levels of inflammatory cytokines with both depression and cachexia, and experiments have shown that introducing cytokines induces depression and cachectic symptoms in both humans and rodents, suggesting that there may be a common etiology at the molecular levelcccxii.

CANCER AND STATINS Statins exert immunomodulatory, anti-inflammatory, anti-angiogenic and anti-proliferative functions by reducing the isoprenylation of proteins involved in cell signal transduction such as Ras and RhoA.

Statins disrupt localization and function of geranylgeranylated proteins responsible for activating signal transduction pathways essential for the growth and/or survival of transformed cells. Exposure of primary and established acute myelogenous leukemia (AML) cells to statins results in significant disruption of basal extracellular signal-regulated kinase (ERK) 1/2 phosphorylation. Statins may trigger apoptosis by regulating several signaling pathways, including the Raf/MEK/ERK pathwaycccxiii. Several natural (lovastatin, simvastatin and pravastatin) and synthetic (cerivastatin and atorvastatin) statins exert a cytotoxic effect on human T, B and myeloma tumor cells by promoting their apoptosis.

One study observed the statin induced reduction of mitochondrial membrane potential and the cytosolic release of the second mitochondria-derived activator of caspases (Smac/DIABLO). The apoptotic pathway was caspase-dependent since caspases 9, 3 and 8 were efficiently activated. Cell proliferation was rescued by both farnesylpyrophosphate (FPP) and geranyl-geranylpyrophosphate (GGPP), whereas no effect was obtained with squalene, a direct precursor of cholesterolcccxiv.

In another study, Atorvastatin and fluvastatin were able to inhibit the proliferation of MCF-7 breast cancer cells in the absence of estradiol. This effect seems to depend on an apoptotic statin effect which may be mediated by the down-regulation of the anti-apoptotic protein Bcl-2 rather than up-regulation of Fas-L or p53. However, in the presence of estradiol the inhibitory effect of the statins was less pronouncedcccxv.

One study examined the effect of a synthetic statin, fluvastatin, on the development of renal cancer. The effects of fluvastatin on cell viability, cell cycle, in vitro angiogenesis, and invasive properties were examined in murine renal cancer cell Renca. The changes in cell cycle-associated proteins, p21(Waf1/Cip1) and p53, and rac1 phosphorylation were analyzed by Western blotting. The prophylactic efficacy of fluvastatin to murine pulmonary metastasis of Renca was examined. Fluvastatin inhibited in vitro growth of Renca cells in a time- and dose-dependent manner, with up to 70% inhibition at a concentration of 10 mumol/L. This inhibitory effect was due to cell cycle arrest at the G(1) phase and induction of apoptosis accompanied by up-regulation of p21(Waf1/Cip1) and p53. The invasive properties of Renca cells through Matrigel were inhibited by fluvastatin, with decreased phosphorylation of rac1. In vitro angiogenesis was also inhibited by fluvastatin. Furthermore, oral administration at doses of 1 to 10 mg/kg/d, for 12 days after inoculation of Renca cells via the tail vein, significantly decreased the amount of pulmonary metastasis. The authors suggested that fluvastatin may effectively inhibit in vitro tumor growth, invasion, angiogenesis, and metastasis of Renca cells, and that oral administration of fluvastatin could be a novel, safe, and effective agent for preventing metastasis of renal cancercccxvi.

Observational studies have shown that Statin use may be associated with reduced cancer risk. One case-control study in patients with prostatic cancer suggested that statins may reduce the risk of total prostate cancer and, specifically, more aggressive prostate cancercccxvii. Another study assessed the effect of statin treatment on a surrogate marker for prostate cancer risk, that is serum prostate specific antigen (PSA), in a cohort of airline pilots from 1992 to 2001. Subject medical records were abstracted for data on age, PSA testing, hyperlipidemia and treatment with statins. The treatment group was composed of 15 men with hypercholesterolemia who received statins and the comparison group of 85 with normal serum lipid levels during the review period. The mean+/−SD and the Wilcoxon rank sum test were used for analyses. Serum PSA was significantly higher in the treatment group at baseline relative to the comparison group (p=0.05). Interestingly there was no significant difference between the groups on subsequent follow-up. There was a 41.6% decrease in mean serum PSA in the treated group by visit 4. Simultaneously mean serum PSA increased by 38% in the untreated group. The authors suggested that treatment with statins may lower serum PSA with timecccxviii.

CANCER AND BISPHOSPHONATES In human epidermoid head and neck KB and lung H1355 cancer cells, 48 h exposure to Pamidronate (PAM) and zoledronic acid (ZOL) induced growth inhibition (IC(50) 25 and 10 microM, respectively) and apoptosis and abolished the proliferative and antiapoptotic stimuli induced by epidermal growth factor (EGF). In these experimental conditions, ZOL induced apoptosis through the activation of caspase 3 and a clear fragmentation of poly(ADP-ribose) polymerase (PARP), was also demonstrated. A strong decrease of basal ras activity and an antagonism on its stimulation by EGF was recorded in the tumor cells exposed to aminobisphosphonates (BPs). These effects were paralleled by impaired activation of the survival enzymes extracellular signal regulated kinase 1 and 2 (Erk-1/2) and Akt that were not restored by EGF. Conversely, famesol induced a recovery of ras activity and antagonized the proapoptotic effects induced by BPscccxix. Bisphosphonates have direct antitumor effects in vivo in addition to their therapeutic antiresorptive properties. Bisphosphonates inhibit proliferation and induce apoptosis of many cancer cell lines. They also exhibit anti-invasive properties in vitro and in vivo. One study investigated the antitumor properties of three nitrogen-containing bisphosphonates on A431 human epidermoid carcinoma cells cells in vitro. The authors first compared the antiproliferative effects of pamidronate, alendronate and neridronate. Then, by matrigel invasion assay, the effect of alendronate on A431 cell invasiveness was studied. All three bisphosphonates were found to inhibit cell proliferation dose- and time-dependentlycccxx. Animal models have shown that bisphosphonates decrease tumor-induced osteolysis and reduce skeletal tumor burden. Zoledronic acid inhibits proliferation and induces apoptosis of human prostate cancer cell lines in vitro and has enhanced antitumor activity when combined with taxanes. In a model of prostate cancer, zoledronic acid significantly inhibited growth of both osteolytic and osteoblastic tumors and reduced circulating levels of prostate-specific antigencccxxi.

Ras proteins are frequently over-expressed in leukemia and contribute to leukemogenesis. In one study, a third-generation bisphosphonate, ONO5920/YM529 (YM529) prevents the prenylation of Ras proteins and inhibited the growth of leukemic cells including a P-glycoprotein (P-gp) over-expressing cell line in a concentration- and time-dependent manner by inducing apoptosis in vitro. YM529 synergistically augmented the anti-leukemic activities of paclitaxel and daunorubicin in vitro and also prolonged the survival of NOD/SCID mice engrafted with human primary leukemic cellscccxxii.

On the basis of results from three large, randomized, phase III clinical trials enrolling more than 3,000 patients, zoledronic acid (4 mg via 15-minute infusion) was approved in the United States for the treatment of patients with documented bone metastases from solid tumors in conjunction with standard antineoplastic therapy and patients with multiple myeloma.

CANCER, PLANT POLYPHENOLS, AND FATTY ACIDS Epidemiological evidence suggests that consumption of soy is associated with a decreased risk for breast, colon, prostate, thyroid, and head and neck cancerscccxxiii. Soy and isoflavone intake are associated with reduced risk of ovarian cancer in southeast Chinacccxxiv. The incidences of breast and prostate cancers are much higher in the United States and European countries compared with Asian countries such as Japan and Chinacccxxv. Frequent consumption of green tea is inversely associated with the risk of several types of human cancer, and studies with animal and in vitro cell culture models have revealed EGCG as a major chemopreventive ingredient of green tea. The lower frequencies of breast and prostate cancer in Asian population in general, compared to those in Western societies have also been attributed to their consumption of relatively large amounts of soy productscccxxvi. Epidemiological studies in human populations and experimental studies in rodents also provide evidence that green tea and its constituents can inhibit both the development and growth of tumors at a variety of tissue sites. In addition, EGCG, a major biologically active component of green tea, inhibits growth and induces apoptosis in a variety of cancer cell lines. These effects are mediated, at least in part, through inhibition of the activity of specific receptor tyrosine kinases (RTKs) and related downstream pathways of signal transduction. The antitumor effects of the related polyphenolic phytochemicals resveratrol, genistein, curcumin, and capsaicin are exerted via similar mechanisms. Some of these agents (EGCG, genistein, and curcumin) appear to directly target specific RTKs, and all of these compounds cause inhibition of the activity of the transcription factors AP-1 and NF-kappaB, thus inhibiting cell proliferation and enhancing apoptosiscccxxvii. Genistein inhibits steroidogenesis and blocks several protein tyrosine kinases, including epidermal growth factor receptor and src tyrosine kinases. Genistein arrests the cell cycle, induces apoptosis, and has anti-angiogenic and anti-metastatic propertiescccxxviii. Genistein inhibits protein tyrosine kinase (PTK), which is involved in phosphorylation of tyrosyl residues of membrane-bound receptors leading to signal transduction, and it inhibits topoisomerase II, which participates in DNA replication, transcription and repair. By blocking the activities of PTK, topoisomerase II and matrix metalloprotein (MMP9) and by down-regulating the expression of about 11 genes, including that of vascular endothelial growth factor (VEGF), genistein can arrest cell growth and proliferation, cell cycle at G2/M, invasion and angiogenesis. Furthermore, genistein can alter the expression of gangliosides and other carbohydrate antigens to facilitate their immune recognition. Genistein acts synergistically with drugs such as tamoxifen, cisplatin, 1,3-bis 2-chloroethyl-1-nitrosourea (BCNU), dexamethasone, daunorubicin and tiazofurin, and with bioflavonoid food supplements such as quercetin, green-tea catechins and black-tea thearubigins. Genistein can augment the efficacy of radiation for breast and prostate carcinomas. Because it increases melanin production and tyrosinase activity, genistein can protect melanocytes of the skin of Caucasians from UV-B radiation-induced melanoma. Genistein-induced antigenic alteration has the potential for improving active specific immunotherapy of melanoma and carcinomas. When conjugated to B43 monoclonal antibody, genistein becomes a tool for passive immunotherapy to target B-lineage leukemias that overexpress the target antigen CD19. Genistein is also conjugated to recombinant EGF to target cancers overexpressing the EGF receptor.

The transcription factor NF-kappa B is elevated in murine T-cell lymphoma lines compared with normal thymic lymphocytes, and may play a role in the neoplastic transformation of these cells. When T lymphoma cells were treated with the soy isoflavone genistein, a marked reduction in nuclear NF-kappa B levels was detectable predominantly for the p50/p50 homodimer and p50/p65 heterodimercccxxix. Although genistein has many potentially therapeutic actions against cancer, its biphasic bioactivity (inhibitory at high concentrations and activating at low concentrations) requires caution in determining therapeutic doses of genistein alone or in combination with chemotherapy, radiation therapy, and/or immunotherapiescccxxx. In one study, genistein was shown to significantly inhibit the growth and induce the apoptosis of human breast cancer MCF-7 cells. Apoptotic cells of morphology from MCF-7 cells treated by different concentrations of genistein were observed by fluorescent and electronic microscope. The frequency of apoptosis in MCF-7 cells by flow cytometry showed increasingly as concentrations of genistein increased. The expression of Bax protein in MCF-7 cells was increased and the expression of erbB-2 protein was decreased with the doses of genisteincccxxxi. Pretreatment with genistein potentiates cell killing induced by radiation in human PC-3 prostate carcinoma cell line. In one study using an orthotopic prostate carcinoma model of PC-3 cells in nude mice, established prostate tumors were pretreated with p.o. genistein at a dose of 5 mg/d for 2 days followed by tumor irradiation with 5 Gy photons. One day after radiation, genistein was resumed and given every other day for 4 weeks. Genistein combined with radiation caused a significantly greater inhibition of primary tumor growth (87%) compared with genistein (30%) or radiation (73%) alone. The number of metastatic lymph nodes was also significantly decreased following genistein and radiation. Paradoxically, genistein alone increased the size of lymph nodes associated with heavy tumor infiltration. Genistein-treated prostate tumors were large with necrosis, apoptotic cells, and giant cells and had a lower proliferation index than in control tumors. Following radiation, areas of tumor destruction replaced by fibrotic tissue and inflammatory cells as well as giant cells were observed, which are typical of radiation effect. After radiation and genistein treatment, an increase in giant cells, apoptosis, inflammatory cells, and fibrosis was observed with decreased tumor cell proliferation consistent with increased tumor cell destruction. The authors concluded that long-term therapy with genistein after prostate tumor irradiation significantly increased survivalcccxxxii.

A microarray was performed to screen 847 genes involved in cytokine signaling, signal transduction, and transcription. Tyrosine kinases represented a common target driving proliferation among the three human pancreatic cancer cell types. Eighteen genes were found to be commonly expressed by the three cell lines. Of these, six (33%) included tyrosine phosphorylation signaling as part of the pathway. The most highly expressed common transcript was the EphB3 receptor, which is a tyrosine kinase. Herbimycin and Genistein were able to inhibit the proliferation of all three cell lines in a dose dependent manner, with a mean IC(50) of 1.71 microM and 223 microM, respectively; whereas Lavendustin and Gleevec were ineffective in the inhibition of proliferationcccxxxiii. Genistein has also been found to inhibit proliferation of a renal cell carcinoma cell line, GRC-1. In one study, inverted microscopy, MTT method, and flow cytometry (FCM) were used to examine the changes in proliferation of GRC-1 cells after treatment with genistein; and the intracellular anti-oncogene, p27 protein expression was determined by Western blot analysis. After treatment with genistein, changed morphology of the GRC-1 cells was observed. Cell junctions decreased. In the presence of 20 micromol/L genistein, GRC-1 cells showed shuttle-shaped, and fewer pseudopodia, mitoses and cell junctions were observed. In the 40 micromol/L genistein group, many cells broke into debris, and became extremely irregular in shape. Meanwhile, mitoses and cell junctions were rarely seen. After treatment with 20 micromol/L genistein, 73.8% of GRC-1 cells were in G(1) phase, 26.2% in G2 phase 72 hours after treatment; while in control group, 31.6% in G(1) phase and 3.8% in G2 phase, respectively. After exposure to 20 micromol/L genistein for 72 hours, Western blot suggested that the band of p27 was 65.4+/−4.7 in gray scale value, while the control group was 52.3+/−6.3. The authors concluded that Genistein can inhibit the proliferation of renal cell carcinoma cells, and cause cell cycle arrest at G(1)/M, G(2)/S phasecccxxxiv. One study examined the effect of green tea polyphenols (GTP) on growth and metastasis of highly metastatic mouse mammary carcinoma 4T1 cells in vitro and in vivo systems. Treatment of 4T1 cells with EGCG resulted in inhibition of cell proliferation, induction of apoptosis in dose- and time-dependent manner. The increase in apoptosis was accompanied with decrease in the protein expression of Bcl-2 concomitantly increase in Bax, cytochrome c release, Apaf-1, and cleavage of caspase 3 and PARP proteins. Treatment of EGCG-rich GTP in drinking water to 4T1 cells bearing BALB/c mice resulted in reduction of tumor growth accompanied with increase in Bax/Bcl-2 ratio, reduction in proliferating cell nuclear antigen and activation of caspase 3 in tumors. Metastasis of tumor cells to lungs was inhibited and survival period of animals was increased after green tea treatmentcccxxxv. Overexpression of the epidermal growth factor receptor family member Her-2/neu in breast cancer is associated with poor prognosis. One study examined the effects of epigallocatechin-3 gallate (EGCG) on Her-2/neu-overexpressing breast cancer cells. EGCG inhibited mouse mammary tumor virus (MMTV)-Her-2/neu NF639 cell growth in culture and soft agar. EGCG reduced signaling via the phosphatidylinositol 3-kinase, Akt kinase to NF-kappaB pathway because of inhibition of basal Her-2/neu receptor tyrosine phosphorylation. EGCG similarly inhibited basal receptor phosphorylation in SMF and Ba/F3 2+4 cellscccxxxvi.

Green tea has shown remarkable anti-inflammatory and cancer chemopreventive effects in many animal tumor bioassays, cell culture systems, and epidemiological studies. Many of these biological effects of green tea are mediated by epigallocatechin 3-gallate (EGCG), the major polyphenol present therein. EGCG treatment has been shown to result in apoptosis of several cancer cells, but not of normal cells (J. Natl. Cancer Inst. 89, 1881-1886 (1997)). The mechanism of this differential response of EGCG is not known. In one study, EGCG treatment resulted in a dose-dependent (i) inhibition of cell growth, (ii) G0/G1-phase arrest of the cell cycle, and (iii) induction of apoptosis in human epidermoid carcinoma (A431) cells, but not in normal human epidermal keratinocytes (NHEK). Electromobility shift assay revealed that EGCG (10-80 microM) treatment results in lowering of NF-kappaB levels in both the cytoplasm and nucleus in a dose-dependent manner in both A431 cells and NHEK, albeit at different concentrationscccxxxvii.

In summary, consumption of plant-derived foods, especially fruits, vegetables, nuts and grains has been linked to decreased risk of cancer. Laboratory studies with animals and cells in culture have shown cancer preventive activity of chemicals isolated from soy, tea, rice and many green, yellow and orange fruits and vegetables. Using cell culture, transgenic mice and knockout mice models to examine the anti-cancer effects of these dietary factors at the molecular level, one study found that (11) (−)-epigallocatechin gallate (EGCG), the major active polyphenol in green tea, and theaflavins, the major active components in black tea, inhibit epidermal growth factor (EGF)- or 12-O-tetradecanoylphorbol-13-acetate (TPA)-induced JB6 cell transformation. At the same dose range that inhibited cell transformation, EGCG and theaflavins inhibited activator protein-1 (AP-1) activation. These compounds also inhibited ultraviolet B (UVB)-induced AP-1 and nuclear factor kappa B (NFkappaB)-dependent transcriptional activation; (2) resveratrol, found at high levels in grapes, inhibited cell transformation through the induction of apoptosis, mediated through JNK and p53-dependent pathways; (3) inositol hexaphosphate (InsP6), an active compound from rice and other grains, inhibited TPA- or EGF-induced transformation and signal transduction through its effects on phosphatidylinositol-3 kinase (PI-3) kinase; (4) phenethyl isothiocyanate (PEITC), which occurs as a conjugate in certain cruciferous vegetables, inhibited cell transformation corresponding with the induction of apoptosiscccxxxviii.

Omega-3 fatty acids (OM-3FAs) have been shown to possess anti-carcinogenic properties. Supplementing the diet of tumor-bearing mice or rats with oils containing (n-3) (omega-3) or with purified (n-3) fatty acids has slowed the growth of various types of cancers, including lung, colon, mammary, and prostate. The efficacy of cancer chemotherapy drugs such as doxorubicin, epirubicin, CPT-11, 5-fluorouracil, and tamoxifen, and of radiation therapy has been improved when the diet included (n-3) fatty acids. In humans, (n-3) fatty acids have also been used to suppress cancer-associated cachexia and to improve the quality of life. In one study, the response to chemotherapy therapy was better in breast cancer patients with higher levels of (n-3) fatty acids in adipose tissue [indicating past consumption of (n-3) fatty acids] than in patients with lower levels of (n-3) fatty acidscccxxxix. One study investigated the effect of OM-3FAs on epithelial ovarian cancer cell lines to determine if a growth suppressive effect existed and to gain a better insight on the potential molecular mechanisms that may be involved. Three epithelial ovarian cancer cell lines (SKOV-3 [p53 null], TOV-21G [wt p53] and OVCAR-3 [mutant p53]) and one immortalized ovarian surface epithelial cell line (IOSE-29 [wt p53]) were treated with OM-3FAs and evaluated for cellular proliferation (WST-1 assay), apoptosis (Annexin V-FITC/PI method) and VEGF expression (VEGF ELISA assay). A statistically significant inhibitory effect under the influence of OM-3FAs was detected in all four cell lines. Apoptosis and VEGF down-regulation were either limited or not detected in the p53 null and mutant cell lines, SKOV-3 and OVCAR-3 respectively. Apoptosis and/or VEGF down-regulation was strongly evident in the wt p53 cell lines TOV-21G and IOSE-29cccxl.

AGING, AGE-RELATED DISORDERS, AND INTERLEUKIN 6 Evidence has linked IL-10 and IL-6 cytokine polymorphisms to longevity. Individuals who are genetically predisposed to produce high levels of IL-6 have a reduced capacity to reach the extreme limits of human life, whereas the high IL-10-producer genotype is increased among centenarianscccxli.

Telomere length is linked to age-associated diseases, with shorter telomeres in blood associated with an increased probability of mortality from infection or heart disease. In patients with multiple myeloma (MM), telomere length (TL) of MM cells is significantly shorter than that of the patients' own leukocytes. In one study, TL negatively correlated with age and with interleukin-6 (IL-6) and beta2-microglobulin levelscccxlii. Overproduction of IL-6, a pro-inflammatory cytokine, is associated with a spectrum of age-related conditions including cardiovascular disease, osteoporosis, arthritis, type 2 diabetes, certain cancers, periodontal disease, frailty, and functional decline. To describe the pattern of change in IL-6 over 6 years among older adults undergoing a chronic stressor, this longitudinal community study assessed the relationship between chronic stress and IL-6 production in 119 men and women who were caregiving for a spouse with dementia and 106 noncaregivers, with a mean age at study entry of 70.58 (SD=8.03) for the full sample. On entry into this portion of the longitudinal study, 28 of the caregivers' spouses had already died, and an additional 50 of the 119 spouses died during the 6 years of this study. Levels of IL-6 and health behaviors associated with IL-6 were measured across 6 years. Caregivers' average rate of increase in IL-6 was about four times as large as that of noncaregivers. Moreover, the mean annual changes in IL-6 among former caregivers did not differ from that of current caregivers even several years after the death of the impaired spouse. There were no systematic group differences in chronic health problems, medications, or health-relevant behaviors that might have accounted for caregivers' steeper IL-6 slope. These data provide evidence of a key mechanism through which chronic stressors may accelerate risk of a host of age-related diseases by prematurely aging the immune response.cccxliii Aortic vascular smooth muscle cells isolated from spontaneously hypertensive rats (SHR) grow nearly twice as fast in vitro as cells isolated from several normotensive control strains of rats. DNA synthesis in SHR cells from both young and adult animals in response to epidermal growth factor is selectively enhanced compared with normotensive controls, suggesting that epidermal growth factor may be at least partly responsible for the enhanced growth rate. One study determined whether the enhanced DNA synthesis in response to epidermal growth factor in SHR cells is mediated via an enhanced epidermal growth factor receptor tyrosine kinase. The researchers measured thymidine incorporation in epidermal growth factor-stimulated vascular smooth muscle cells in the presence of the highly specific tyrosine kinase inhibitor genistein. The 50% inhibitory dose (IC50) of genistein was higher for the SHR vascular smooth muscle cells than for the normotensive Wistar rat (NBR; National Institutes of Health Black rat). The researchers suggest that the increased DNA synthesis in response to epidermal growth factor in SHR cells is a result of higher receptor tyrosine kinase activity initiating further intracellular signalscccxliv. Interleukin-6 is also a causative factor in other manifestations of aging.

Wrinkles on the skin are a manifestation of aging. Excess sunlight, smoking, and exposure to wind, heat, and harsh chemicals causes the outer layers of the skin to thicken and cause skin to wrinkle, sag and become leathery. Ultraviolet (UV) radiation from the sun is widely considered as a major cause of human skin photoaging and skin cancer. IL-6 is produced by keratinocytes in vivo and in vitro and the release is enhanced by UV light. A study was performed to investigate the effect of a single UV dose eliciting moderate to severe sunburn reaction on the production of IL-6 in vivo. Plasma of UV-treated human subjects was evaluated for IL-6 activity by testing its capacity to induce the proliferation of an IL-6-dependent hybridoma cell line (B9). In contrast to plasma samples obtained before UV exposure, post-UV-specimens contained significant levels of IL-6 peaking at 12 h after UV irradiation. Plasma IL-6 activity was neutralized by an antiserum directed against recombinant human IL-6cccxlv. UV radiation-induced proinflammatory cytokines mediated by NF-kappaB reportedly play important roles in sunburn, skin damage, premature aging, and increases the risk of developing melanomas and other types of skin cancer. In one study, immunohistochemical and Western blot analysis and ELISA indicated that both nuclear p65 and secreted IL-6 were significantly (p<0.05) induced by UVB (20, 30 mJ/cm2) and UVA irradiation (10, 20 J/cm2). NF-kappaB nuclear translocation and IL-6 secretion induced by UVB and UVA were dramatically inhibited by treatment of EGCGcccxlvi. Higher levels of the systemic inflammatory markers CRP and IL-6 are independently associated with progression of age-related macular degeneration (AMD). One study tested the hypothesis that baseline cardiovascular disease biomarkers are associated with subsequent increased risk for progression of AMD. This prospective cohort study involved 251 participants aged 60 years and older who had some sign of nonexudative AMD and visual acuity of 20/200 or better in at least one eye at baseline. The AMD status was assessed by standardized grading of fundus photographs, and stored fasting blood specimens obtained at baseline were analyzed for levels of the various biomarkers. The average follow-up time was 4.6 years. Comparing the highest quartile with the lowest quartile, CRP was associated with progression of AMD, with a multivariate adjusted relative risk (RR) of 2.10 (95% confidence interval [CI], 1.06-4.18; P for trend, 0.046) controlling for body mass index, smoking, and other cardiovascular variables and a multivariate adjusted RR of 2.02 (95% CI, 1.00-4.04; P for trend, 0.06) controlling additionally for antioxidant nutrients. Interleukin 6 was also related to progression of AMD, with a multivariate adjusted RR of 1.81 (95% CI, 0.97-3.36; P for trend, 0.03). Comparing the highest quartile with the lowest quartile, the effect estimates for vascular cell adhesion molecule 1 (multivariate adjusted RR, 1.94) and apolipoprotein B (adjusted RR, 1.39) were in the positive direction but were not statistically significant (P for trend, 0.08 and 0.24, respectively). The CRP and IL-6 levels were both significantly related to higher body mass index and current smokingcccxlvii.

AGING, AGE-RELATED DISORDERS AND STATINS Considering the role of Interleukin-6 mediated inflammation in aging, age-related disorders and/or age-related manifestations including Atherosclerosis, Peripheral Vascular Disease, Coronary Artery Disease, Osteoporosis, Type 2 Diabetes, Dementia and Alzheimer's disease and some forms of Arthritis and Cancer, statins should play an important role in the prevention and treatment of aging, age-related disorders and/or age-related manifestations.

AGING, AGE-RELATED DISORDERS AND BISPHOSPHONATES Considering the role of Interleukin-6 mediated inflammation aging, age-related disorders and/or age-related manifestations including Atherosclerosis, Peripheral Vascular Disease, Coronary Artery Disease, Osteoporosis, Type 2 Diabetes, Dementia and Alzheimer's disease and some forms of Arthritis and Cancer, bisphosphonates should play an important role in the prevention and treatment of aging, age-related disorders and/or age-related manifestations.

AGING, AGE-RELATED DISORDERS AND FOOD POLYPHENOLS Considering the role of Interleukin-6 mediated inflammation in aging, age-related disorders and/or age-related manifestations including Atherosclerosis, Peripheral Vascular Disease, Coronary Artery Disease, Osteoporosis, Type 2 Diabetes, Dementia and Alzheimer's disease and some forms of Arthritis and Cancer, synthetic or plant derived polyphenolic compounds found in fruits, vegetables, nuts, grains, cereals, dry legumes, chocolate, and beverages, such as tea, coffee, or wine should play a vital role in the prevention and treatment of aging, age-related disorders and/or age-related manifestations.

AGING, AGE-RELATED DISORDERS, IL-6 AND GENE THERAPY/MODULATION Genetic polymorphisms involving a change of a single base, from guanine to cytosine, at position—174 in the 5′ flanking region of the interleukin-6 gene is of great importance because the G allele is associated with higher IL-6 production than the C allele. In vivo studies have found basal IL-6 levels to be twice as high in volunteers with the GG allele than in those with the CC allele. The polymorphism in the 5′ flanking region, (an area important in the regulation of gene expression) alters the transcriptional response to stimuli such as LPS and IL-1cccxlviii. An increased frequency of an Xba I Restriction Fragment Length Polymorphism (RFLP, likely to be due to 3′ flanking region insertions, has been described in some patients with SLE and elevated IL-6 levelscccxlix. By using polymerase chain reaction (PCR)-RFLP and sensitive polyacrylamide gel electrophoresis, an association between genotype for the 3′ flanking region polymorphism and peak bone mineral density in women has been demonstratedcccl. Manipulating the genetic mechanisms controlling the IL-6 levels and increasing the frequency of GG alleles in the population would prevent aging and age related diseases and be the key to eternal youth and immortality. Gene therapy will aim to provide for targeted gene transfer, controlled expression of the gene transferred and enhanced activity of the transferred gene product. An alternate means of gene therapy is gene modulation. In gene modulation, expression of an already expressed gene is increased by introducing exogenous normal genetic sequences and decreased by introducing antisense genes or gene fragments, or by introducing vectors that can produce ribozymes that can cleave specific mRNAs. Gene modulation can also be achieved by the introduction of exogenous normal genetic sequences that code for proteins that modulate the extent of gene expression, or affect the processing, assembly or secretion of gene products.

CLINICAL IMPLICATIONS OF CHRONIC INHIBITION OF IL-6-MEDIATED INFLAMMATION There are currently no large clinical studies utilizing combination of statins, bisphosphonates, and/or synthetic or plant derived polyphenolic compounds to synergistically inhibit Interleukin-6 mediated inflammation. There have been large clinical studies utilizing either statins or bisphosphonates. Some of the patients in these studies may have been on both statins and bisphosphonates. Evidence of safety and efficacy of combination treatment with statins and bisphosphonates may be sought from new clinical trials or sub-group analyses or meta-analyses of existing studies.

The statin studies have shown that statins may decrease the progression of coronary artery diseasecccli ccclii, reduce the risks of heart attack and deathcccliii cccliv ccclv ccclvi ccclvii ccclviii lower the risk of stroke in people with coronary artery diseaseccclix. The Prospective Pravastatin Pooling Project (PPP) looked at the long-term safety and efficacy of statins in secondary prevention, based on pooled results from three key statin trials. PPP revealed a highly significant relative risk reduction in total mortality, fatal and nonfatal coronary events, and stroke events in patients with a broad range of patient characteristicsccclx. The trial demonstrated that pravastatin has a similar incidence of muscle-related side effects as placeboccclxi. The Collaborative AtoRvastatin Diabetes Study (CARDS) showed patients with type 2 diabetes who received atorvastatin 10 mg daily for four years had a 37% relative risk reduction in the primary endpoint (acute coronary heart disease death, fatal or non-fatal myocardial infarction, unstable angina requiring hospital admission, resuscitated cardiac arrest, coronary revascularisation procedures and stroke)ccclxii. The trial was terminated over a year early on account of a clear benefit demonstrated for the intervention group. Numerous large-scale clinical trials have consistently demonstrated a positive safety and tolerability profile for statins. Non-life-threatening side effects may occur in up to 15% of patients receiving one statin. More serious side effects that may require discontinuation of statin therapy may also occur but at much lower rates. These include significant elevations in the activity of serum aminotransferase and creatine kinase alone or in combination with muscle painccclxiii. The safety of statins in children and adolescents has not yet been well documented.

Bisphosphonates are widely used in osteoporosis and other bone diseases. Large clinical trials have established the strong safety and tolerability profile of bisphosphonatesccclxiv ccclxv. In the Fracture Intervention Trial (FIT)ccclxvi ccclxvii, administration of alendronate to postmenopausal women with low femoral bone mineral density (BMD) increased spinal BMD to 8 percent over baseline, with a 50 percent decrease in the risk of new vertebral, hip and wrist fractures in women with at least one preexisting vertebral fracture at baseline. The bisphosphonates have minimal non-skeletal toxicity because they bind to bone and are not taken up by other tissuesccclxviii. The reduction in renal function that occurs in animal models with administration of high-dosage parenteral bisphosphonate has not occurred in clinical practice. However, because bisphosphonates are excreted through glomerular filtration, intravenous administration of large dosages of pamidronate to patients with severe chronic renal failure or patients on dialysis may be accompanied by marked hypocalcemia and/or hypophosphatemia with associated tetany.39 Iritis, muscle aches and fever can also accompany intravenous bisphosphonate administration and is reversible on discontinuation. Oral bisphosphonates seem to induce serious esophagitis in some patients, may result in gastritis and cause diarrhea.40. When used as recommended, serious esophageal complications are few. Patients with known esophageal disease (e.g., achalasia, stricture, Barrett's esophagus, severe reflux and scleroderma) should avoid taking oral bisphosphonates.

CONCLUSION In conclusion, we have described the biochemical pathway from cholesterol synthesis to interleukin 6 mediated inflammation. It is our theory that Interleukin 6 mediated inflammation is the gatekeeper and common causative factor for aging, age-related disorders and/or age-related manifestations including Atherosclerosis, Peripheral Vascular Disease, Coronary Artery Disease, Osteoporosis, Type 2 Diabetes, Dementia and Alzheimer's disease and some forms of Arthritis and Cancer. We have clarified the relationship between these common illnesses and we determine that pleiotropic effects of bisphosphonates, statins and polyphenolic compounds are mediated by inhibition of Interleukin 6 mediated inflammation.

Isoprenoids, which are intermediates, generated in the cholesterol biosynthesis pathway, play a more significant role than the end product cholesterol, in activation of Interleukin 6 mediated inflammation. Isoprenoids are generated by endogenous cellular cholesterol synthesis in the body as well as by cholesterol synthesis in activated monocytes during the inflammatory response. However, isoprenoids are but one component of the signaling pathway for Interleukin 6 mediated inflammation.

Inhibition of the signal transduction pathway for Interleukin 6 mediated inflammation is key to the prevention and treatment of aging, age-related disorders and/or age-related manifestations including atherosclerosis, peripheral vascular disease, coronary artery disease, osteoporosis, type 2 diabetes, dementia, Alzheimer's disease and some forms of arthritis and cancer. Inhibition of Interleukin 6 mediated inflammation may be achieved indirectly through regulation of endogenous cholesterol synthesis and isoprenoid depletion or by direct inhibition of the signal transduction pathway utilizing interleukin-6 inhibitor/antibody, interleukin-6 receptor inhibitor/antibody, interleukin-6 antisense oligonucleotide (ASON), gp130 protein inhibitor/antibody, tyrosine kinases inhibitors/antibodies, serine/threonine kinases inhibitors/antibodies, mitogen-activated protein (MAP) kinase inhibitors/antibodies, phosphatidylinositol 3-kinase (PI3K) inhibitors/antibodies, Nuclear factor κB (NF-κB) inhibitors/antibodies, IκB kinase (IKK) inhibitors/antibodies, activator protein-1 (AP-1) inhibitors/antibodies, STAT transcription factors inhibitors/antibodies, altered IL-6, partial peptides of IL-6 or IL-6 receptor, or SOCS (suppressors of cytokine signaling) protein, PPAR gamma and/or PPAR beta/delta activators/ligands or a functional fragment thereof.

Prompt treatment of infection such as in periodontal disease will prevent or reduce the generation of isoprenoids and induction of Interleukin-6 mediated inflammation by activated monocytes.

Statins, Bisphosphonates and Polyphenolic Compounds have similar mechanisms of action and act on similar diseases in the following ways:

1. Statins and Bisphosphonates inhibit the Mevalonate to Cholesterol conversion pathway and cause isoprenoid depletion; with inhibition of interleukin-6 inflammation. Statins inhibit the enzyme HMG-CoA reductase and Bisphosphonates inhibit the enzyme FPP Synthase. Polyphenolic Compounds inhibit multiple pathways of signal transduction for Interleukin 6 mediated inflammation including inhibition of tyrosine kinase activity, inhibition of activation of NF-κB and inhibition of activation of IKK complex.

2. Statins, Bisphosphonates and Polyphenolic Compounds inhibit the JAK/STAT3 signaling pathway for Interleukin 6 mediated inflammation.

3. Statins, Bisphosphonates and Polyphenolic Compounds have common pleiotropic effects and decrease the progression of atherosclerotic vascular disease and inhibit bone resorption.

4. Combination treatment with agents that inhibit different aspects of the signal transduction pathways for interleukin 6 mediated inflammation, including Statins, Bisphosphonates and Polyphenolic Compounds, will be transformational and have better efficacy with fewer side effects in the prevention and treatment of aging, age-related disorders and/or age-related manifestations including atherosclerosis, peripheral vascular disease, coronary artery disease, osteoporosis, type 2 diabetes, dementia and some forms of arthritis and tumors. Evidence of safety and efficacy of combination treatment with inhibitors of Interleukin-6 mediated inflammation should be sought from new clinical trials.

Statins, Bisphosphonates are just indirect inhibitors of Interleukin-6 inflammation but yet both class of drugs have enabled a significant decrease in mortality and morbidity from these common illnesses.

Epidemiological evidence suggests that increased consumption of plant derived polyphenolic compounds is associated with decrease in mortality and morbidity from these common illnesses. Newer therapies and drugs will be interleukin-6 inhibitor/antibody, interleukin-6 receptor inhibitor/antibody, interleukin-6 antisense oligonucleotide (ASON), gp130 protein inhibitor/antibody, tyrosine kinases inhibitors/antibodies, serine/threonine kinases inhibitors/antibodies, mitogen-activated protein (MAP) kinase inhibitors/antibodies, phosphatidylinositol 3-kinase (PI3K) inhibitors/antibodies, Nuclear factor κB (NF-κB) inhibitors/antibodies, IκB kinase (IKK) inhibitors/antibodies, activator protein-1 (AP-1) inhibitors/antibodies, STAT transcription factors inhibitors/antibodies, altered IL-6, partial peptides of IL-6 or IL-6 receptor, or SOCS (suppressors of cytokine signaling) protein, PPAR gamma and/or PPAR beta/delta activators/ligands or a functional fragment thereof.

The public health significance of such new drugs will be transformational.

It will be apparent to those skilled in the art that variations and modifications to the specific embodiments disclosed herein may be made without departing from the scope of the invention.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.
ⁱ Am J Cardiol 2000; 85: 10A-17A.
Rationale and design of the Cardiac Hospitalization Atherosclerosis Management Program at the University of California Los Angeles.
Fonarow G C, Gawlinski A.
ⁱⁱ Hackam D G, Anand S S.
Emerging risk factors for atherosclerotic vascular disease: a critical review of the evidence.
JAMA. 2003 Aug. 20; 290(7):932-40
ⁱⁱⁱ Ross R.
Atherosclerosis an inflammatory disease.
N Engl J Med 1999; 340:115-26
^{iv} Libby P, Ridker P M, Maser A.
Inflammation and atherosclerosis.
Circulation 2002; 105:1135-43.
^{v} Ross, R. 1995
Cell biology of atherosclerosis.
Annu. Rev. Physiol. 57:791-804.
^{vi} Tabas, I. 1997.
Free cholesterol-induced cytotoxicity. A possible contributing factor to macrophage foam cell necrosis in advanced atherosclerotic lesions.
Trends Cardiovasc. Med. 7:256-263
^{vii} Kockx, M. M. 1998.
Apoptosis in the atherosclerotic plaque: quantitative and qualitative aspects.
Arterioscler. Thromb. Vasc. Biol. 18:1519-1522.
^{viii} Mitchinson, M. J., Hardwick, S. J., and Bennett, M. R.
Cell death in atherosclerotic plaques.
Curr. Opin. Lipidol. 7:324-329. 1996.
^{ix} Davignon J, Mabile L.
Mechanisms of action of statins and their pleiotropic effects
[Article in French]
Ann Endocrinol (Paris). 2001 February; 62(1 Pt 2):101-12
^{x} Endocrine. 2004 February; 23(1):1-10
Osteoporosis and cardiovascular disease: brittle bones and boned arteries, is there a link?
McFarlane S I, Muniyappa R, Shin J J, Bahtiyar G, Sowers J R.
xi Interleukin-6 and the acute phase response
Heinrich P C, Castell J V, Andus T.
Biochem J 1990; 265:621-636
^{xii} Elevated levels of interleukin-6 in unstable angina.
Biasucci L M, Vitelli A, Liuzzo G, et al.
Circulation 1996; 94:874-877
^{xiii} Biasucci L M, Liuzzo G, Fantuzzi G, et al.
Increasing levels of interleukin (IL)-1Ra and IL-6 during the first 2 days of hospitalization in unstable angina are associated with increasing risk of in-hospital coronary events.
Circulation 1999; 99:2079-2084
^{xiv} Szekanecz Z, Shah M R, Pearce W H, Koch A E
Human atherosclerotic abdominal aortic aneurysms produce interleukin (IL)-6 and interferon-gamma but not IL-2 and IL-4: the possible role for IL-6 and interferon-gamma in vascular inflammation.
Agents Actions 1994; 42:159-162
^{xv} Ridker P M, Rifai N, Pfeifer M, et al.
Elevation of tumor necrosis factor-alpha and increased risk of recurrent coronary events after myocardial infarction.
Circulation 2000; 101:2149-2153
^{xvi} Ridker P M, Rifai N, Stampfer M J, Hennekens C H.
Plasma concentration of interleukin-6 and the risk of future myocardial infarction among apparently healthy men.
Circulation 2000; 101: 1767-1772
^{xvii} Ma J, Hennekens C H, Ridker P M, Stampfer M J.
A prospective study of fibrinogen and risk of myocardial infarction in the Physicians' Health Study.
J Am Coll Cardiol 1999; 33:1347-1352
xviii. Berk B C, Weintraub W S, Alexander R W
Elevation of C-reactive protein in “active” coronary artery disease.
Am J Cardiol 1990; 65:168-172.
^{xix} Ford E S, Giles W H.
Serum C-reactive protein and fibrinogen concentrations and self-reported angina pectoris and myocardial infarction: findings from National Health and Nutrition Examination Survey III.
J Clin Epidemiol 2000; 53:95-102
^{xx} Morrow D A, Ridker P M.
C-reactive protein, inflammation, and coronary risk.
Med Clin North Am 2000; 84:149-161
^{xxi} Ridker P M, Rifai N, Pfeifer M A, et al.
Long-term effects of pravastatin on plasma concentration of C-reactive protein. The Cholesterol and Recurrent Events (CARE) Investigators.
Circulation 1999; 100:230-235
^{xxii} Schaumberg D A, Ridker P M, Glynn R J, et al.
High levels of plasma C-reactive protein and future risk of age-related cataract.
Ann Epidemiol 1999; 9:166-171
^{xxiii} Ridker P M, Cushman M, Stampfer M J, et al.
Plasma concentration of C-reactive protein and risk of developing peripheral vascular disease.
Circulation 1998; 97:425-428
^{xxiv} Cesari M, Penninx B W, Newman A B, Kritchevsky S B, Nicklas B J, Suiton-Tyrrell K, Rubin S M, Ding J, Simonsick E M, Harris T B, Pahor M
Inflammatory markers and onset of cardiovascular events: results from the Health ABC study.
Circulation. 2003 Nov. 11; 108(19):2317-22.
^{xxv} Zhao L, Hart S, Cheng J, Melenhorst J J, Bierie B, Ernst M, Stewart C, Schaper F, Heinrich P C, Ullrich A, Robinson G W, Hennighausen L.
Mammary gland remodeling depends on gp130 signaling through Stat3 and MAPK.
J Biol. Chem. 2004 Oct. 15; 279(42):44093-100. Epub 2004 Jul. 30
^{xxvi} Cesari M, Penninx B W, Newman A B, Kritchevsky S B, Nicklas B J, Sufton-Tyrrell K, Tracy R P, Rubin S M, Harris T B, Pahor M
Inflammatory markers and cardiovascular disease (The Health, Aging and Body Composition [Health ABC] Study).
Am J Cardiol. 2003 Sep. 1; 92(5):522-8
^{xxvii} Yaffe K, Lindquist K, Penninx B W, Simonsick E M, Pahor M, Kritchevsky S, Launer L, Kuller L, Rubin S, Harris T.
Inflammatory markers and cognition in well-functioning African-American and white elders.
Neurology 2003 Jul. 8; 61(1):76-80
^{xxviii} J Immunol 144, 4226-4230 (1990)
IL-6 stimulates osteoclast-like multinucleated cell formation in long term human marrow cultures by inducing IL-1 release.
N. Kurihara, D. Bertolini, T. Suda, Y. Akiyama & G. D. Roodman:
^{xxix} Kumaki, T. Taga, T. Kishimoto & T. Suda:
Soluble interleukin-6 receptor triggers osteoclast formation by interleukin-6.
T. Tamura, N. Udagawa, N. Takahashi, C. Miyaura, S. Tanaka, Y. Koishihara, Y. Ohsugi, K.
Proc Natl Acad Sci USA 90, 11924-11928 (1993)
^{xxx} Manolagas S C, Jilka R L.
Bone marrow, cytokines, and bone remodeling. Emerging insights into the pathophysiology of osteoporosis.
N Engl J Med. 1995 Feb. 2; 332(5):305-11.
^{xxxi} Ravaud P, Thepot C, Auleley G R, Amor B.
Imaging of multiple myeloma.
Ann Med Interne [Paris] 1996; 147:370-5.
^{xxxii} Teoh G, Anderson K C.
Interaction of tumor and host cells with adhesion and extracellular matrix molecules in the development of multiple myeloma.
Hematol Oncol Clin North Am 1997; 11:27-42
^{xxxiii} S. S. Tabibzadeh, U. Santhanam, P. B. Sehgal & L. T. May
Cytokine-induced production of IFN-β2/IL-6 by freshly explanted human endometrial stromal cells. Modulation by estradiol-17B.
J Immunol 142, 3134-3139 (1989)
^{xxxiv} D. M. Kania, N. Binkley, M. Checovich, T. Havighurst, M. Schilling & W. B. Ershler
Elevated plasma levels of interleukin-6 in postmenopausal women do not correlate with bone density.
J Am Geriat Soc 43, 236-239 (1995)
^{xxxv} R. A. Daynes, B. A. Araneo, W. B. Ershler, C. Maloney, G. Z. Li & S. Y. Ryu
Altered regulation of IL-6 production with normal aging. Possible linkage to the age-associated decline in dehydroepiandrosterone and its sulfated derivative.
J Immunol 150, 5219-5230 (1993)
^{xxxvi} R. L. Jilka, C. Hangoc, G. Girasole, G. Passeri, D. C. Williams, J. S. Abrams, B. Boyce, H. Broxmeyer & S. C. Manolagas
Increased osteoclast development after estrogen loss: Mediation by interleukin-6.
Science 257, 88-91 (1992)
^{xxxvii} S. Kotake, K. Sato, K. J. Kim, N. Takahashi, N. Udagawa, I. Nakamura, A. Yamaguchi, T. Kishimoto, T. Suda & S. Kashiwazaki
Interleukin-6 and soluble interleukin-6 receptors in the synovial fluids form rheumatoid arthritis patients are responsible for osteoclast-like cell formation.
J Bone Miner Res 11, 88-95 (1996)
^{xxxviii} F. Houssiau, J. P. Devoglaer, J. Van Damme, C. Nagant de Deuxchaisnes & J. Van
Snick: Interleukin 6 in synovial fluid and serum of patients with rheumatoid arthritis and other inflammatory arthritides.
Arthritis Rheum 31, 784-788 (1988)
^{xxxix} Yoshii T, Magara S, Miyai D, Nishimura H, Kuroki E, Furudoi S, Komori T, Ohbayashi C.
Local levels of interleukin-1beta, -4, -6 and tumor necrosis factor alpha in an experimental model of murine osteomyelitis due to staphylococcus aureus.
Cytokine. 2002 Jul. 21; 19(2):59-65.
^{xl} Meghji S, Crean S J, Hill P A, Sheikh M, Nair S P, Heron K, Henderson B, Mawer E B, Harris M.
Surface-associated protein from Staphylococcus aureus stimulates osteoclastogenesis: possible role in S. aureus-induced bone pathology.
Br J Rheumatol. 1998 October; 37(10):1095-101.
^{xli} Hotokezaka H, Kitamura A, Matsumoto S, Hanazawa S, Amano S, Yamada T.
Internalization of Mycobacterium bovis Bacillus Calmette-Guerin into osteoblast-like MC3T3-E1 cells and bone resorptive responses of the cells against the infection.
Scand J Immunol. 1998 May; 47(5):453-8
^{xlii} Kiecolt-Glaser J K, Preacher K J, MacCallum R C, Atkinson C, Malarkey W B, Glaser R.
Chronic stress and age-related increases in the proinflammatory cytokine IL-6.
Proc Natl Acad Sci USA. 2003 Jul. 22; 100(15):9090-5. Epub 2003 Jul. 2.
^{xliii} Lutgendorf S K, Garand L, Buckwalter K C, Reimer T T, Hong S Y, Lubaroff D M.
Life stress, mood disturbance, and elevated interleukin-6 in healthy older women.
J Gerontol A Biol Sci Med Sci. 1999 September; 54(9):M434-9.
^{xliv} Paukku K, Silvennoinen O.
STATs as critical mediators of signal transduction and transcription: lessons learned from STAT5.
Cytokine Growth Factor Rev. 2004 December; 15(6):435-55.
^{xlv} Heinrich P C, Behrmann I, Muller-Newen G, Schaper F, Graeve L.
Interleukin-6-type cytokine signalling through the gp130/Jak/STAT pathway.
Biochem J. 1998 Sep. 1; 334 (Pt 2):297-314.
^{xlvi} Darnell J E.
STATs and gene regulation.
Science 1998; 277: 1630-1635
^{xlvii} Ogata A, Nishimoto N, Yoshizaki K
Advances in interleukin-6 therapy.
Rinsho Byori. 1999 April; 47(4):321-6.
^{xlviii} Hirano T, Nakajima K, Hibi M
Signaling mechanisms through gp130: a model of the cytokine system.
Cytokine Growth Factor Rev. 1997 December; 8(4):241-52.
^{xlix} Akira S
IL-6-regulated transcription factors.
Int J Biochem Cell Biol. 1997 December; 29(12):1401-18.
^{l} Yu C L, Meyer D J, Campbell G S, et al.
Enhanced DNA-binding activity of a Stat3-related protein in cells transformed by the Src oncoprotein.
Science. 1995 Jul. 7; 269 (5220): 81-3
^{li} Leukoc Biol. 1999 October; 66(4):588-92.
Suppressors of cytokine signaling (SOCS): negative regulators of signal transduction.
Alexander W S, Starr R, Metcalf D, Nicholson S E, Farley A, Elefanty A G, Brysha M, Kile B T, Richardson R, Baca M, Zhang J G, Willson T A, Viney E M, Sprigg N S, Rakar S, Corbin J, Mifsud S, DiRago L, Cary D, Nicola N A, Hilton D J.
^{lii} Nature. 1997 Jun. 26; 387(6636):924-9
Structure and function of a new STAT-induced STAT inhibitor.
Naka T, Narazaki M, Hirata M, Matsumoto T, Minamoto S, Aono A, Nishimoto N, Kajita T, Taga T, Yoshizaki K, Akira S, Kishimoto T.
^{liii} Nature. 1997 Jun. 26; 387(6636):924-9.
Structure and function of a new STAT-induced STAT inhibitor.
Naka T, Narazaki M, Hirata M, Matsumoto T, Minamoto S, Aono A, Nishimoto N, Kajita T, Taga T, Yoshizaki K, Akira S Kishimoto T.
^{liv} Yu R Y, Wang X, Pixley F J, Yu J J, Dent A L, Broxmeyer H E, Stanley E R, Ye B H.
BCL-6 negatively regulates macrophage proliferation by suppressing autocrine IL-6 production.
Blood. 2005 Feb. 15; 105(4):1777-84. Epub 2004 Oct. 26.
^{lv} Karin M. (1995)
The regulation of AP-1 activity by mitogen-activated protein kinases.
J. Biol. Chem. 270:16483-6.
^{lvi} Sen R, Baltimore D 1986
Multiple nuclear factors interact with the immunoglobulin enhancer sequences
Cell 46:706-716
^{lvii} Fujita T, Nolan G P, Ghosh S, Baltimore D 1992
Independent modes of transcriptional activation by the p50 and p65 subunits of NF-κβ.
Genes Dev 6:775-787-
^{lviii} Zhong H, SuYang H, Erdjument-Bromage H, Tempst P, Ghosh S 1997
The transcriptional activity of NF-κβ is regulated by the I-κβ-associated PKAc subunit through a cyclic AMP-independent mechanism.
Cell 89:413-424
^{lix} Verma I, Stevenson J K, Schwarz E M, Van Antwerp D, Miyamoto S 1995
Rel/NFκβ/Iκβ family: intimate tales of association and dissociation.
Genes Dev 9:2723-2735
^{lx} Chen Z, Parent L, Maniatis T 1996
Site-specific phosphorylation of Iκβ by a novel ubiquitination-dependent pathway
Cell 84:853-862
^{lxi} Baeuerle P A, Henkel T 1994
Function and activation of NF-κB 1995 Rel/NF T 1996 Site-specific phosphorylation of Tin vitro cell mediated immune assay predicts in vivo response.
J Rheumatol 18:1130-1133
^{lxii} Verma I, Stevenson J K, Schwarz E M, Van Antwerp D, Miyamoto S 1995
Rel/NFκβ/Iκβ family: intimate tales of association and dissociation.
Genes Dev 9:2723-2735
^{lxiii} Riothwarf D M, Zanid E, Natoli G, Karin M.
IKK-gamma is an essential regulatory subunit of the IkappaB kinase complex.
Nature. 1998 Sep. 17; 395(6699):297-300.
^{lxiv} Alkalay I, Yaron A, Hatzubai A, Orian A, Ciechanover A, Ben-Neriah Y 1995
Stimulation-dependent Iκβ phosphorylation marks the NFκβ inhibitor for degradation via the ubiquitin-proteasome pathway.
Proc Natl Acad Sci USA 92:10599-10603
^{lxv} Palombella V J, Rando O J, Goldberg A L, Maniatis T 1994
The ubiquitin-proteasome pathway is required for processing the NF-κβ1 precursor protein and the activation of NF-κβ.
Cell 78:773-785
^{lxvi} Baldwin A S 1996
The NFκB and IκB proteins: new discoveries and insights.
Annu Rev Immunol 14:649-681
^{lxvii} Adcock I M, Lane S J, Brown C R, Lee T H, Barnes P J 1995
Abnormal glucocorticoid receptor-activator protein 1 interaction in steroid-resistant asthma.
J Exp Med 182:1951-1958-
^{lxviii} Lorraine I. McKay and John A. Cidlowski
Cross-Talk between Nuclear Factor-κB and the Steroid Hormone Receptors: Mechanisms of Mutual Antagonism
Molecular Endocrinology 12 (1): 45-56
^{lxix} Zhong H, SuYang H, Erdjument-Bromage H, Tempst P, Ghosh S 1997
The transcriptional activity of NF-κB is regulated by the I-κβ-associated PKAc subunit through a cyclic AMP-independent mechanism.
Cell 89:413-424
^{lxx} Karin M, Ben-Neriah Y. (2000)
Phosphorylation meets ubiquitination: the control of NF-κB activity.
Annu. Rev. Immunol. 18:621-63.
^{lxxi} Adv Exp Med Biol. 2003; 544:181-96.
A paradigm for gene regulation: inflammation, NF-kappaB and PPAR.
Vanden Berghe W, Vermeulen L, Delerive P, De Bosscher K, Staels B, Haegeman G.
^{lxxi} Akira S, Kishimoto T.
IL-6 and NF-IL6 in acute-phase response and viral infection.
Immunol Rev. 1992; 127:25-50
^{lxxiii} Loppnow H, Libby P.
Proliferating or interleukin-1 activated human vascular smooth muscle cells secrete copious IL-6.
J Clin Invest. 1990; 85:731-738
^{lxxiv} Han Y, Runge M S, Brasier A R.
Angiotensin II induces interleukin-6 transcription in vascular smooth muscle cells through pleiotropic activation of nuclear factor-kappa B transcription factors.
Cic Res. 1999 Apr. 2; 84(6):695-703.
^{lxxv} Curr Drug Targets Inflamm Allergy. 2002 September; 1(3):243-8.
Peroxisome proliferator-activated receptors and the control of inflammation.
Cabrero A, Laguna J C, Vazquez M.
^{lxxvi} Biochem Pharmacol. 2000 Oct. 15; 60(8):1245-50
Role of the peroxisome proliferator-activated receptors (PPAR) in atherosclerosis.
Neve B P, Fruchart J C, Staels B.
^{lxxvii} King M. W.
Medical Biochemistry Course Guide, 2004
Cholesterol and Bile Metabolism
Indiana University School of Medicine,
^{lxxviii} Leung K C.
Regulation of cytokine receptor signaling by nuclear hormone receptors: a new paradigm for receptor interaction.
DNA Cell Biol. 2004 August; 23(8):463-74.
^{lxxix} Das U. N.
Statins and the prevention of dementia
CMAJ. Oct. 2, 2001; 165 (7)
^{lxxx} Physiol Rev. 2001 January; 81(1):153-208
Small GTP-binding proteins.
Takai Y, Sasaki T, Matozaki T
^{lxxxi} J Cell Sci. 2005 Mar. 1; 118(Pt 5):843-6
The Ras superfamily at a glance.
Wennerberg K, Rossman K L, Der C J
lxxxii Magee T, Marshall C.
New insights into the interaction of Ras with the plasma membrane.
Cell 1999; 98: 9-12
^{lxxxiii} Dechend R., Müller D., Park J. K., Fiebeler A., Haller H., Luft F. C.
Statins and angiotensin II-induced vascular injury
Nephrol Dial Transplant (2002) 17: 349-353
^{lxxxiv} Clin Oncol. 1999 March; 17(3):1071-9.
RAS and leukemia: from basic mechanisms to gene-directed therapy.
Beaupre D M, Kurzrock R
^{lxxxv} Biochem Biophys Res Commun. 2000 Mar. 24; 269(3):798-802.
Autocrine/Paracrine secretion of IL-6 family cytokines causes angiotensin II-induced delayed STAT3 activation.
Sano M, Fukuda K, Kodama H, Takahashi T, Kato T, Hakuno D, Sato T, Manabe T, Tahara S, Ogawa S.
^{lxxxvi} Faruqi T R, Gomez D, Bustelo X R, Bar-Sagi D, Reich N C
Rac1 mediates STAT3 activation by autocrine IL-6.
Proc Natl Acad Sci USA. 2001 Jul. 31; 98(16):9014-9. Epub 2001 Jul. 24.
^{lxxxvii} Mol Biol Cell. 2001 October; 12(10):3282-94
Simultaneous tyrosine and serine phosphorylation of STAT3 transcription factor is involved in Rho A GTPase oncogenic transformation.
Aznar S, Valeron P F, del Rincon S V, Perez L F, Perona R, Lacal J C.
^{lxxxviii} Lubbert M, Oster W, Knopf H P, McCormick F, Mertelsmann R, Herrmann F
N-RAS gene activation in acute myeloid leukemia: association with expression of interleukin-6.
Leukemia 1993 December; 7(12):1948-54
^{lxxxix} Leukemia. 2002 September; 16(9):1664-7.
Farnesyl transferase inhibitor R115777 induces apoptosis of human myeloma cells.
Le Gouill S, Pellat-Deceunynck C, Harousseau J L, Rapp M J, Robillard N, Bataille R, Amiot M.
^{xc} Mol Cell Biol. 2003 February; 23(4):1316-33
Rho family GTPases are required for activation of Jak/STAT signaling by G protein-coupled receptors.
Pelletier S, Duhamel F, Coulombe P, Popoff M R, Meloche S.
^{xci} R Terkeltaub, J Solan, M Barry, D Santoro and G M Bokoch
Role of the mevalonate pathway of isoprenoid synthesis in IL-8 generation by activated monocytic cells
Journal of Leukocyte Biology, 1994 June; Vol 55, Issue 6 749-755
^{xcii} Hillyard D Z, Jardine A G, McDonald K J, Cameron A J.
Fluvastatin inhibits raft dependent Fcgamma receptor signalling in human monocytes.
Atheosclerosis. 2004 February; 172(2):219-28
^{xciii} Pahan K, Sheikh F G, Namboodiri A M, Singh I
Lovastatin and phenylacetate inhibit the induction of nitric oxide synthase and cytokines in rat primary astrocytes, microglia, and macrophages.
J Clin Invest. 1997 Dec. 1; 100(11):2671-9
^{xciv} Dubey vs
Mevalonate-independent pathway of isoprenoids synthesis:
A potential target in some human pathogens
Current science, vol. 83, no. 6, 25 Sep. 2002
^{xcv} Burkhart C N, Gottwald L
Assessment of etiologic agents in acne pathogenesis.
Skinmed. 2003 July-August; 2(4):222-8.
^{xcvi} Vinayak Kapatral, lain Anderson, Natalia Ivanova, Gary Reznik, Tamara Los, Athanasios Lykidis, Anamitra Bhattacharyya, Allen Bartman, Warren Gardner, Galina Grechkin, Lihua Zhu, Olga Vasieva, Lien Chu, Yakov Kogan, Oleg Chaga, Eugene Goltsman, Axel Bernal, Niels Larsen, Mark D'Souza, Theresa Walunas, Gordon Pusch, Robert Haselkorn, Michael Fonstein, Nikos Kyrpides, and Ross Overbeek
Genome Sequence and Analysis of the Oral Bacterium Fusobacterium nucleatum Strain ATCC 25586
Journal of Bacteriology, April 2002, p. 2005-2018, Vol. 184, No. 7
^{xcvii} Mattila K J, Nieminen M S, Valtonen V W, Rasi V P, Kesaniemi Y A, Syrjala S L, Jungell P S, Isoluoma M, Hietaniemi K, Jokinen M J.
Association between dental health and acute myocardial infarction.
BMJ. 1989 Mar. 25; 298(6676):779-81.
^{xcviii} Morrison H I, Ellison L F, Taylor G W.
Periodontal disease and risk of fatal coronary heart and cerebrovascular diseases.
J Cardiovasc Risk. 1999 February; 6(1):7-11
Oral Meyer D H, Fives-Taylor P M.
Oral pathogens: from dental plaque to cardiac disease.
Curr Opin Microbiol. 1998 February; 1(1):88-95.
^{c} Desvarieux M.
Periodontal disease, race, and vascular disease.
Compend Contin Educ Dent. 2001 July; 22(3 Spec No):34-41
^{ci} Champagne C M, Madianos P N, Lieff S, Murtha A P, Beck J D, Offenbacher S.
Periodontal medicine: emerging concepts in pregnancy outcomes.
J Int Acad Periodontol. 2000 January; 2(1):9-13.
^{cii} Assuma R, Oates T, Cochran D, Amar S, Graves D T.
IL-1 and TNF antagonists inhibit the inflammatory response and bone loss in experimental periodontitis.
J Immunol. 1998 Jan. 1; 160(1):403-9
^{ciii} Treatment of Periodontal Disease in Diabetics Reduces Glycated Hemoglobin
Sara G. Grossi, Fred B. Skrepcinski, Thomas DeCaro, Don C. Robertson, Alex W. Ho, Robert G. Dunford, and Robert J. Genco
Journal of Periodontology: August 1997 Vol. 68, No. 8: 713-9.
^{civ} Prenylation inhibitors in renal disease.
Khwaja A, O'Connolly J, Hendry B M
Lancet 2000; 355: 741-744
^{cv} Ikeda U, Shimpo M, Ohki R K et al.
Fluvastatin inhibits matrix metalloproteinase-1 expression in human vascular endothelial cells.
Hypertension 2000; 36: 325-329
^{cvi} Bergstrom J D, Bostedor R G, Masarachia P J, Reszka A A, Rodan G.
Alendronate is a specific, nanomolar inhibitor of farnesyl diphosphate synthase.
Arch Biochem Biophys. 2000 Jan. 1; 373(1):231-41
^{cvii} Rogers M J.
New insights into the molecular mechanisms of action of bisphosphonates.
Curr Pharm Des. 2003; 9(32):2643-58
^{cviii} Montagnani A, Gonnelli S, Cepollaro C, Campagna M S, Franci M B, Pacini S, Gennari C.
Changes in serum HDL and LDL cholesterol in patients with Paget's bone disease treated with pamidronate.
Bone. 2003 January; 32(1):15-9.
^{cix} Manzoni M, Rollini M.
Biosynthesis and biotechnological production of statins by filamentous fungi and application of these cholesterol-lowering drugs.
Appl Microbiol Biotechnol. 2002 April; 58(5):555-64. Epub 2002 Feb. 14.
^{cx} Manach C, Scalbert A, Morand C, Remesy C, Jimenez L.
Am J Clin Nutr. 2004 May; 79(5):727-47.
Polyphenols: food sources and bioavailability.
^{cxi} Akiyama et al.
Genistein, a specific inhibitor of tyrosine-specific protein kinases
J Biol Chem. 1987 Apr. 25; 262(12):5592-5
^{cxii} Calder P C.
Dietary modification of inflammation with lipids.
Proc Nutr Soc 2002; 61:345-358
^{cxiii} Symposium Highlights—Omega-3 Fatty Acids: Recommendations for Therapeutics and Prevention
Medscape General Medicine 7(4):18, 2005.
^{cxiv} Mantzioris E, James M J, Gibson R A, Cleland L G.
Dietary substitution with an alphalinolenic acid-rich vegetable oil increase eicosapentaenoic acid concentrations in tissues.
Am J Clin Nutr 1994; 59:1304-1309
^{cxv} Dc Lorgeril M, Renaud S, Mamelle N, et at.
Mediterranean alpha-linolenic acid-rich diet in the secondary prevention of coronary heart disease. Lancet 1994; 343:1454-1459.
^{cxvi} Meydani S N, Endres S, Woods, M M, et al.
Oral (n-3) fatty acid supplementation suppresses cytokine production and lymphocyte proliferation: comparison between young and older women.
^{cxvii} Kremer J M. Studies of dietary supplements with omega-3 fatty acids in patients with rheumatoid arthritis. In: Nelson G J, Ed. Health Effects of Dietary Fatty Acids.
Champaign, Ill.: American Oil Chemists' Society, pp223-233, 1991
^{cxviii} Sundrarjun T, Komindr S, Archararit N, Dahlan W, Puchaiwatananon O, Angthararak S, Udomsuppayakul U, Chuncharunee S.
Effects of n-3 fatty acids on serum interleukin-6, tumour necrosis factor-alpha and soluble tumour necrosis factor receptor p55 in active rheumatoid arthritis.
J Int Med Res. 2004 September-October; 32(5):443-54.
^{cxix} Hypertension. 1999 July; 34(1):118-25
Induction of interleukin-6 expression by angiotensin II in rat vascular smooth muscle cells.
Funakoshi Y, Ichiki T, Ito K, Takeshita A.
^{cxx} Axterioscler Thromb Vasc Biol. 2001 September; 21(9):1464-9
Angiotensin II administration to atherosclerotic mice increases macrophage uptake of oxidized Id1: a possible role for interleukin-6.
Keidar S, Heinrich R, Kaplan M, Hayek T, Aviram M.
^{cxxi} Circulation. 2000 Apr. 18; 101(15):1799-805
Enzymatically degraded, nonoxidized LDL induces human vascular smooth muscle cell activation, foam cell transformation, and proliferation.
Klouche M, Rose-John S, Schmiedt W, Bhakdi S.
^{cxxii} J Immunol. 1999 Oct. 15; 163(8):4583-9
Novel path to activation of vascular smooth muscle cells: up-regulation of gp130 creates an autocrine activation loop by IL-6 and its soluble receptor.
Klouche M, Bhakdi S, Hemmes M, Rose-John S.
^{cxxiii} Biochem Int. 1990; 20(3):445-53
Interleukin-6 stimulates c-myc expression and proliferation of cultured vascular smooth muscle cells.
Nabata T, Morimoto S, Koh E, Shiraishi T, Ogihara T.
^{cxxiv} Cytokine. 1994 January; 6(1):87-91
Interleukin 6 gene transcripts are expressed in human atherosclerotic lesions.
Seino Y, Ikeda U, Ikeda M, Yamamoto K, Misawa Y, Hasegawa T, Kano S, Shimada K.
^{cxxv} Arterioscler Thromb Vasc Biol. 2001 November; 21(11):1759-63
Thrombin induces interleukin-6 expression through the cAMP response element in vascular smooth muscle cells.
Tokunou T, Ichiki T, Takeda K, Funakoshi Y, Iino N, Shimokawa H, Egashira K, Takeshita A.
^{cxxvi} Williams N, Bertoncello I, Jackson H, Arnold J, Kavnoudias H.
The role of interleukin 6 in megakaryocyte formation, megakaryocyte development and platelet production.
Ciba Found Symp. 1992; 167:160-170; discussion 170-173
^{cxxvii} Oleksowicz L, Mrowiec Z, Zuckerman D, Isaacs R, Dutcher J, Puszkin E.
Platelet activation induced by interleukin-6: evidence for a mechanism involving arachidonic acid metabolism.
Thromb Haemost. 1994; 72:302-308
^{cxxviii} Burstein S A.
Effects of interleukin 6 on megakaryocytes and on canine platelet function.
Stem Cells. 1994; 12:386-393
^{cxxix} Am J Nephrol. 2004 July-August; 24(4):387-92. Epub 2004 Jul. 9
Role of JAK/STAT pathway in IL-6-induced activation of vascular smooth muscle cells.
Watanabe S, Mu W, Kahn A, Jing N, Li J H, Lan H Y, Nakagawa T, Ohashi R, Johnson R J.
^{cxxx} Res Commun Mol Pathol Pharmacol. 2001 March-April; 109(3-4):241-8.
A new immunological marker of atherosclerotic injury of arterial wall.
Okopien B, Hyper M, Kowalski J, Belowski D, Madej A, Zielinski M, Tokarz D, Kalina Z, Herman Z S
^{cxxxi} Atherosclerosis. 2003 August; 169(2):283-91.
Soluble intercellular adhesion molecule-1 and interleukin-6 levels reflect endothelial dysfunction in patients with primary hypercholesterolaemia treated with atorvastatin.
Nawawi H, Osman N S, Annuar R, Khalid B A, Yusoff K.
^{cxxxii} Aust Prescriber. 2000; 23(6):130-2.
Experimental and clinical pharmacology: bisphosphonates-mechanisms of action.
Martin T J G, V.
^{cxxxiii} Med. 1997 Oct. 20; 115(29):37-42.
[Bisphosphonate therapy in osteoporosis. Inhibition of trabecular perforation by aminobisphosphonate]. Fortschr
Wuster C, Heilmann P.
^{cxxxiv} Clin Exp Rheumatol. 2002 May-June; 20(3):359-64
Clodronate treatment reduces serum levels of interleukin-6 soluble receptor in Paget's disease of bone.
Rendina D, Postiglione L, Vuotto P, Numis F G, Di Domenico G, Viceconti R, Mossetti G, Nunziata V
^{cxxxv} Methods Find Exp Clin Pharmacol. 1999 October; 21(8):519-22.
Etidronate inhibits the production of IL-6 by osteoblast-like cells.
Olmos J M, De Vega T, Perera L, Riancho J A, Amado J A, Gonzalez-Macias J.
^{cxxxvi} Scand J Rheumatol. 1998; 27(1):38-41.
Bisphosphonates inhibit IL-6 production by human osteoblast-like cells.
Giuliani N, Pedrazzoni M, Passeri G, Girasole G.
^{cxxxvii} Endocrinology. 1996 June; 137(6):2324-33
Bisphosphonates induce osteoblasts to secrete an inhibitor of osteoclast-mediated resorption.
Vitte C, Fleisch H, Guenther H L.
^{cxxxviii} J Clin Invest. 1999 November; 104(10):1363-74
Prevention of osteocyte and osteoblast apoptosis by bisphosphonates and calcitonin.
Plotkin L I, Weinstein R S, Parfitt A M, Roberson P K, Manolagas S C, Bellido T
^{cxxix} Eur J Haematol. 1998 August; 61(2):128-34-
Long-term oral pamidronate treatment inhibits osteoclastic bone resorption and bone turnover without affecting osteoblastic function in multiple myeloma.
Abildgaard N, Rungby J, Glerup H, Brixen K, Kassem M, Brincker H, Heickendorff L, Eriksen E F, Nielsen J L
^{cxl} Chen X, Garner S C, Quarles L D, Andersojl J J.
Effects of genistein on expression of bone markers during MC3T3-E1 osteoblastic cell differentiation.
J Nutr Biochem. 2003 June; 14(6):342-9.
^{cxli} Kim B H, Chung E Y, Ryu J C, Jung S H, Min K R, Kim Y.
Anti-inflammatory mode of isoflavone glycoside sophoricoside by inhibition of interleukin-6 and cyclooxygenase-2 in inflammatory response.
Arch Pharm Res 2003 April; 26(4):306-11.
^{cxlii} Suh K S, Koh G, Park C Y, Woo J T, Kim S W, Kim J W, Park I K, Kim Y S.
Soybean isoflavones inhibit tumor necrosis factor-alpha-induced apoptosis and the production of interleukin-6 and prostaglandin E2 in osteoblastic cells.
Phytochemistry. 2003 May; 63(2):209-15.
^{cxliii} Borsellino N, Bonavida B, Ciliberto G, Toniatti C, Travali S, D'Alessandro N.
Blocking signaling through the Gp130 receptor chain by interleukin-6 and oncostatin M inhibits PC-3 cell growth and sensitizes the tumor cells to etoposide and cisplatin-mediated cytotoxicity.
Cancer. 1999 Jan. 1; 85(1):134-44.
^{cxliv} Jones T H, Justice S K, Price A.
Suppression of tyrosine kinase activity inhibits [3H]thymidine uptake in cultured human pituitary tumor cells.
J Clin Endocrinol Metab. 1997 July; 82(7):2143-7.
^{cxlv} Geng Y, Zhang B, Lotz M.
Protein tyrosine kinase activation is required for lipopolysaccharide induction of cytokines in human blood monocytes.
J Immuno. 1993 Dec. 15; 151(12):6692-700.
^{cxlvi} Lin J K, Liang Y C, Lin-Shiau S Y. (1999)
Cancer chemoprevention by tea polyphenols through mitotic signal transduction blockade.
Biochem. Pharmacol.
58:911-5
^{cxlvii} Pan M H, Lin-Shiau S Y, Ho C T, Lin J H, Lin J K. (2000)
Suppression of lipopolysaccharide-induced nuclear factor-kappaB activity by theaflavin-3,3.-digallate from black tea and other polyphenols through down-regulation of IkappaB kinase activity in macrophages.
Biochem. Pharmacol 59:357-67-
^{cxlviii} Wang Z Y et al. (1994)
Inhibitory effects of black tea, green tea, decaffeinated black tea, and decaffeinated green tea on ultraviolet B light-induced skin carcinogenesis in 7,12-dimethylbenz[a]anthracene-initiated SKH-1 mice.
Cancer Res. 54:3428-35.
^{cxlix} Yang F, Oz H S, Barve S, de Villiers W J, McClain C J, Varilek G W.
The green tea polyphenol (−)-epigallocatechin-3-gallate blocks nuclear factor-kappa B activation by inhibiting I kappa B kinase activity in the intestinal epithelial cell line IEC-6.
Mol Pharmacol. 2001 September; 60(3):528-33.
^{cl} Rajesh Aneja, Paul W Hake, Timothy J Burroughs, Alvin G Denenberg, Hector R Wong, And Basilia Zingarelli
Epigallocatechin, a Green Tea Polyphenol, Attenuates Myocardial Ischemia Reperfusion Injury in Rats
Molecular Medicine, January-June 2004, Vol 10, No 1-6
^{cli} Dona M et al. (2003)
Neutrophil restraint by green tea: Inhibition of inflammation, associated angiogenesis, and pulmonary fibrosis.
J. Immunol. 170:4335-41.
^{clii} Levites Y, Amit T, Youdim M B, Mandel S. (2002)
Involvement of protein kinase C activation and cell survival/cell cycle genes in green tea polyphenol (−)-epigallocatechin 3-gallate neuroprotective action.
J. Biol. Chem. 277:30574-80
^{cliii} Levites Y, Youdim M B, Maor G, Mandel S. (2002)
Attenuation of 6-hydroxydopamine (6-OHDA)-induced nuclear factor-kappaB (NF-kappaB) activation and cell death by tea extracts in neuronal cultures.
Biochem. Pharmacol. 63:21-9.
^{cliv} Ahmad N, Feyes D K, Nieminen A L, Agarwal R, Mukhtar H. (1997)
Green tea constituent epigallocatechin-3-gallate and induction of apoptosis and cell cycle arrest in human carcinoma cells.
J. Natl. Cancer Inst 89:1881-6.
^{clv} Tedeschi E, Suzuki H, Menegazzi M.
Antiinflammatory action of EGCG, the main component of green tea, through STAT-1 inhibition.
Ann N Y Acad. Sci. 2002 November; 973:435-7.
^{clvi} Watson J L, Ansari S, Cameron H, Wang A, Akhtar M, McKay D M.
Green tea polyphenol (−)-epigallocatechin gallate blocks epithelial barrier dysfunction provoked by IFN-gamma but not by IL-4.
Am J Physiol Gastrointest Liver Physiol. 2004 November; 287(5):G954-61. Epub 2004 Jul. 1.
^{clvii} Levites Y, Amit T, Youdim M B, Mandel S. (2002) Involvement of protein kinase C activation and cell survival/cell cycle genes in
^{green} tea polyphenol (−)-epigallocatechin 3-gallate neuroprotective action.
J. Biol. Chem. 277:30574-80
clViii Levites Y, Amit T, Mandel S, Youdim M B. (2003)
Neuroprotection and neurorescue against Abeta toxicity and protein kinase C dependent release of nonamyloidogenic soluble precursor protein by green tea polyphenol (−)-epigallocatechin-3-gallate.
FASEB J. 17:952-4.
^{clix} Kim H S et al. (2004) EGCG blocks tumor promoter-induced MMP-9 expression
^{via} suppression of MAPK and AP-1 activation in human gastric AGS cells.
Anticancer Res. 24:747-53.
^{clx} Tedeschi E, Suzuki H, Menegazzi M. (2002)
Antiinflammatory action of EGCG, the main component of green tea, through STAT-1 inhibition.
Ann. N.Y. Acad. Sci. 973:435-7.
^{clxi} Chen P C et al. (2002)
A green tea derived polyphenol, epigallocatechin-3-gallate,
^{inhibits} IkappaB kinase activation and IL-8 gene expression in respiratory
epithelium.
Inflammation 26:233-41
^{clxi} Anejea R, Hake P W, Burroughs T J, Denenberg A G, Wong H R, Zingarelli B.
Epigallocatechin, a Green Tea Polyphenol, Attenuates Myocardial Ischemia Reperfusion Injury in Rats
Mol Med-2004 January-June; 10(1-6):55-62
^{clxiii} Mackenzie G G, Carrasquedo F, Delfino J M, Keen C L, Fraga C G, Oteiza P I.
Epicatechin, catechin, and dimeric procyanidins inhibit PMA-induced NF-kappaB activation at multiple steps in Jurkat T cells.
FASEB J. 2004 January; 18(1):167-9. Epub 2003 Nov. 20
^{clxiv} Aneja R, Hake P W, Burroughs T J, Denenberg A G, Wong H R, Zingarelli B.
Epigallocatechin, a Green Tea Polyphenol, Attenuates Myocardial Ischemia Reperfusion Injury in Rats
Mol Med. 2004 January-June; 10(1-6):55-62
^{clxv} Amarakoon A M, Tappia P S, Grimble R F.
Endotoxin induced production of interleukin-6 is enhanced by vitamin E deficiency and reduced by black tea extract.
Inflamm Res. 1995 July; 44(7):301-5.
^{clxvi} Abbate R, Gori A M, Martini F, Brnnelli T, Filippini M, Francalanci I, Paniccia R, Prisco D, Gensini G F, Neri Semeri G G.
n-3 PUFA supplementation, monocyte PCA expression and interleukin-6 production.
Prostaglandins Leukot Essent Fatty Acids. 1996 June; 54(6):439-44
^{clxvii} Khalfoun B, Thibault F, Watier H, Bardos P, Lebranchu Y.
Docosahexaenoic and eicosapentaenoic acids inhibit in vitro human endothelial cell production of interleukin-6.
Adv Exp Med Biol 1997; 400B:589-97
clXviii Sundrarjun T, Komindr S, Archararit N, Dahlan W, Puchaiwatananon O, Angthararak S, Udomsuppayakul U, Chuncharunee S.
Effects of n-3 fatty acids on serum interleukin-6, tumour necrosis factor-alpha and soluble tumour necrosis factor receptor p55 in active rheumatoid arthritis.
J Int Med Res. 2004 September-October; 32(5):443-54.
^{clxix} Nelson T L, Hickey M S.
Acute changes in dietary omega-3 fatty acid intake lowers soluble interleukin-6 receptor in healthy adult normal weight and overweight males.
Cytokine. 2004 Jun. 7; 26(5):195-201
^{clx} Circulation. 2002 Oct. 15; 106(16):2055-60.
ARBITER: Arterial Biology for the Investigation of the Treatment Effects of Reducing Cholesterol: a randomized trial comparing the effects of atorvastatin and pravastatin on carotid intima medial thickness.
Taylor A J, Kent S M, Flaherty P J, Coyle L C, Markwood T T, Vernalis M N.
^{clxxi} Am J. Med. 2004 Mar. 22; 116 Suppl 6A:31S-36S.
Atherosclerosis measured by B-mode ultrasonography: effect of statin therapy on disease progression.
Kastelein J J, de Groot E, Sankatsing R.
^{clxxii} Circulation. 74:1399-1406
Intimal plus medial thickness of the arterial wall: a direct measrement with ultrasound
imaging.
Pignoli P, Tremoli E, Poli A, Oreste P, Paoletti R. 1986
^{clxxiii} Circulation. 87(Suppl 2):II56-II65.
Ultrasound B-mode imaging in observational studies of atherogenic progression.
Salonen J T, Salonen R. 1993
^{clxxiv} Diabetes Care. 17:1330-1336
Common carotid arterial wall thickness in NIDDM subjects.
Pujia A, Gnasso A, Iraace C, Colonna A, Mattioli P L. 1994
^{clxxv} Lancet. 350:SI14-SI19
Non-invasive assessment of cardiovascular disease in diabetes mellitus.
Lehmann E D, Riley W A, Clarkson P, Gosling R G. 1997
^{clxxvi} Diabetes Care. 21:323-324
1998 Carotid arterial intimal-medial thickening and plaque formation in NIDDM [Letter].
Minamikawa J, Yamauchi M, Tanaka S, Koshiyama H.
^{clxxvii} The Journal of Clinical Endocrinology & Metabolism Vol. 85, No. 8 2793-2796
Decrease in Carotid Intima-Media Thickness after 1-Year Therapy with Etidronate for Osteopenia Associated with Type 2 Diabetes
Hiroyuki Koshiyama, Yoshio Nakamura Satsuki Tanaka and Jun Minamikawa
^{clxxviii} Atherosclerosis. 1987 September; 67(1):41-8
The effect of ethane-1-hydroxy-1,1-diphosphonate (EHDP) on necrosis of atherosclerotic lesions.
Daoud A S, Frank A S, Jarmolych J, Fritz K E
clxxix Maziere C, Conte M A, Maziere J C.
Activation of JAK2 by the oxidative stress generated with oxidized low-density lipoprotein.
Free Radic Biol Med. 2001 Dec. 1; 31(11):1334-40.
^{clxxx} J Korean Med Sci. 2004 October; 19 (5):656-61
^{clxxxi} Funakoshi Y, Ichiki T, Ito K, Takeshita A.
Induction of interleukin-6 expression by angiotensin II in rat vascular smooth muscle cells.
Hypertension. 1999 July; 34(1): 118-25.
^{clxxxii} Ruetten H, Thiemermann C.
Endothelin-1 stimulates the biosynthesis of tumour necrosis factor in macrophages: ET-receptors, signal transduction and inhibition by dexamethasone.
J Physiol Pharmacol. 1997 December; 48(4):675-88
^{clxxxiii} Browatzki M, Schmidt J, Kubler W, Kranzhofer R.
Endothelin-1 induces interleukin-6 release via activation of the transcription factor NF-kappaB in human vascular smooth muscle cells.
Basic Res Cardiol. 2000 April; 95(2):98-105.
^{clxxxiv} J Atheroscler Thromb. 2005; 12(1):20-8.
Suppressive effect of cocoa powder on atherosclerosis in Kurosawa and Kusanagi-hypercholesterolemic rabbits.
Kurosawa T, Itoh F, Nozaki A, Nakano Y, Katsuda S, Osakabe N, Tsubone H, Kondo K, Itakura H.
^{clxxxv} Di Yi Jun Yi Da Xue Xue Bao. 2004 September; 24(9):975-9.
[Green tea polyphenols inhibit low-density lipoprotein-induced proliferation of rat vascular smooth muscle cells]
[Article in Chinese]
Ouyang P, Peng W L, Lai W Y, Xu A L.
^{clxxxvi} Atherosclerosis. 2001 May; 156(1):67-72.
Red wine, dealcoholized red wine, and especially grape juice, inhibit atherosclerosis in a hamster model.
Vinson J A, Teue K, Wu N.
^{clxxxvii} De Caterina R, Zampolli A.
n-3 fatty acids: antiatherosclerotic effects.
Lipids. 2001; 36 Suppl:S69-78
^{clxxxviii} Brousseau M E, Schaefer E J.
Diet and coronary heart disease: clinical trials.
Curr Atheroscler Rep. 2000 November; 2(6):487-93.
^{clxxxix} [No authors listed]
Clinical application of omega-3-fatty acids (cod-liver oil)
Klin Med (Mosk). 2005; 83(10):51-7.
^{cxc} Hino A, Adachi H, Toyomasu K, Yoshida N, Enomoto M, Hiratsuka A, Hirai Y, Satoh A, Imaizumi T.
Very long chain N-3 fatty acids intake and carotid atherosclerosis: an epidemiological study evaluated by ultrasonography.
Atherosclerosis. 2004 September; 176(1): 145-9.
^{cxc} Mol Genet Metab. 2004 June; 82(2):180-6.
A common functional variant in the interleukin-6 gene is associated with increased body
^{mass} index in subjects with type 2 diabetes mellitus.
Stephens J W, Hurel S J, Cooper J A, Acharya J, Miller G J, Humphries S E.
^{cxcii} Biochem Biophys Res Commun. 2002 Sep. 20; 297(2):419-24.
Incadronate disodium inhibits advanced glycation end products-induced angiogenesis in vitro.
Okamoto T, Yamagishi S, Inagaki Y, Amano S, Takeuchi M, Kikuchi S, Ohno S, Yoshimura A.
^{cxciii} Yamagishi S, Abe R, Inagaki Y, Nakamura K, Sugawara H, Inokuma D, Nakamura H, Shimizu T, Takeuchi M, Yoshimura A, Bucala R, Shimizu H, Imaizumi T.
Minodronate, a newly developed nitrogen-containing bisphosphonate, suppresses melanoma growth and improves survival in nude mice by blocking vascular endothelial growth factor signaling. Am J Pathol. 2004 December; 165(6):1865-74.
^{cxciv} Diabetologia. 2001 November; 44(11):2032-7.
Bisphosphonates in the treatment of Charcot neuroarthropathy: a double-blind randomised controlled trial.
Jude E B, Selby P L, Burgess J, Lilleystone P, Mawer E B, Page S R, Donohoe M, Foster A V, Edmonds M E, Boulton A J.
^{cxcv} [No authors listed]
A coronary primary prevention study of Scottish men aged 45-64 years: trial design. The West of Scotland Coronary Prevention Study Group.
J Clin Epidemiol. 1992 August; 45(8):849-60.
^{cxcvi} Wong V, Stavar L, Szeto L, Uffelman K, Wang C H, Fantus I G, Lewis G F.
Atorvastatin induces insulin sensitization in Zucker lean and fatty rats.
Atherosclerosis. 2005 Jul. 2
^{cxcvii} Suzuki M, Kakuta H, Takahashi A, Shimano H, Tada-Iida K, Yokoo T, Kihara R, Yamada N.
Effects of atorvastatin on glucose metabolism and insulin resistance in KK/Ay mice.
J Atheroscler Thromb 2005; 12(2):77-84.
^{cxcviii} Lancet. 2004 Aug. 21; 364(9435):685-96.
Primary prevention of cardiovascular disease with atorvastatin in type 2 diabetes in the Collaborative Atorvastatin Diabetes Study (CARDS): multicentre randomised placebo-controlled trial.
Colhoun H M, Betteridge D J, Durrington P N, Hitman G A, Neil H A, Livingstone S J, Thomason M J, Mackness M I, Charlton-Menys V, Fuller J H; CARDS investigators.
^{cxcix} American Journal of Clinical Nutrition, Vol. 76, No. 6, 1191-1201, December 2002
Beneficial role of dietary phytoestrogens in obesity and diabetes
Sam J Bhathena and 4an t T Velasquez
^{cc} Jayagopal, V., Albertazzi, P., Kilpatrick, E. S., Howarth, E. M., Jennings, P. E., Hepburn,
D. A. & Atkin, S. L. (2002) Beneficial effects of soy phytoestrogen intake in postmenopausal women with Type 2 diabetes. Diabetes Care 25:1709-1714
^{cci} Bhathena, S. J. & Velasquez, M. T. (2002)
Beneficial role of dietary phytoestrogens in obesity and diabetes.
Am. J. Clin. Nutr. 76:1191-1201.
^{ccii} J. Nutr. 133:1238-1243, May 2003
Soy Isoflavones Exert Antidiabetic and Hypolipidemic Effects through the PPAR Pathways in Obese Zucker Rats and Murine RAW 264.7 Cells
Orsolya Mezei, William J. Banz*, Richard W. Steger*, Michael R. Peluso*, Todd A. Winters* and Neil Shay
^{cciii} Chinetti, G., Fruchart, J. C. & Staels, B. (2000)
Peroxisome proliferator-activated receptors (PPARs): nuclear receptors at the crossroads between lipid metabolism and inflammation.
Inflamm. Res. 49:497-505
^{cciv} Neve, B. P., Fruchart, J. C. & Staels, B. (2000)
Role of the peroxisome proliferator-activated receptors (PPAR) in atherosclerosis.
Biochem. Pharmacol. 60:1245-1250
^{ccv} Nagy, L., Tontonoz, P., Alvarez, J. G. A., Chen, H. & Evans, R. M. (1998)
Oxidized LDL regulates macrophage gene expression through ligand activation of PPARγ.
Cell 93:229-240.
^{ccvi} Am J Physiol Regul Integr Comp Physiol. 2005 May; 288(5):R1220-5. Epub 2004 Dec. 16.
Adiponectin inhibits LPS-induced NF-kappaB activation and IL-6 production and increases PPARgamma2 expression in adipocytes.
Ajuwon K M, Spurlock M E.
^{ccvii} Invest Ophthalmol Vis Sci. 2001 August; 42(9):2110-4.
Normalization of retinal vascular permeability in experimental diabetes with genistein.
Nakajima M, Cooney M J, Tu A H, Chang K Y, Cao J, Ando A, An G J, Melia M, de Juan E Jr..
^{ccviii} Med Hypotheses. 2005; 64(3):628-35.
Potential utility of natural polyphenols for reversing fat-induced insulin resistance.
McCarty M F.
^{ccix} J Agric Food Chem. 2004 Jan. 14; 52(1):65-70.
Isolation and characterization of polyphenol type-A polymers from cinnamon with insulin-like biological activity.
Anderson R A, Broadhurst C L, Polansky M M, Schmidt W F, Khan A, Flanagan V P, Schoene N W, Graves D J.
^{ccx} Shimura T, Miura T, Usami M, Ishihara E, Tanigawa K, Ishida H, Seino Y.
Docosahexanoic acid (DHA) improved glucose and lipid metabolism in KK-Ay mice with genetic non-insulin-dependent diabetes mellitus (NIDDM).
Biol Pharm Bull. 1997 May; 20(5):507-10.
^{ccxi} Huang Y J, Fang V S, Juan C C, Chou Y C, Kwok C F, Ho L T.
Amelioration of insulin resistance and hypertension in a fructose-fed rat model with fish oil supplementation.
Metabolism. 1997 November; 46(11): 1252-8
^{ccxii} Omoigui S
The Biochemical Origin of Pain: How a new law and new drugs have led to a medical breakthrough in the treatment of Persistent pain.
Hawthorne, Calif. State-of-the-Art Technologies Publishers 2004
^{ccxiii} Veldman P H, Reynen H M, Arntz I E, Goris R J.
Signs and symptoms of reflex sympathetic dystrophy: prospective study of 829 patients.
Lancet 1993 Oct. 23; 342(8878):1012-6
^{ccxiv} Manolagas S C, Jilka R L.
Bone marrow, cytokines, and bone remodeling. Emerging insights into the pathophysiology of osteoporosis.
N Engl J Med. 1995 Feb. 2; 332(5):305-11.
^{ccxv} Roodman G D.
Osteoclast function in Paget's disease and multiple myeloma.
Bone. 1995; 17:57S-61S
^{ccxvi} Ravaud P, Thepot C, Auleley G R, Amor B.
Imaging of multiple myeloma.
Ann Med Interne [Paris] 1996; 147:370-5.
^{ccxvii} Teoh G, Anderson K C.
Interaction of tumor and host cells with adhesion and extracellular matrix molecules in the development of multiple myeloma.
Hematol Oncol Clin North Am 1997; 11:27-42-
Teoh G, Anderson K C.
Interaction of tumor and host cells with adhesion and extracellular matrix molecules in the development of multiple myeloma.
Hematol Oncol Clin North Am 1997; 11:27-42
^{ccxix} S. S. Tabibzadeh, U. Santhanam, P. B. Sehgal & L. T. May
Cytokine-induced production of IFN-β2/IL-6 by freshly explanted human endometrial stromal cells. Modulation by estradiol-17β.
J Immunol 142, 3134-3139 (1989)
^{ccxx} Elevated plasma levels of interleukin-6 in postmenopausal women do not correlate with bone density.
D. M. Kania, N. Binkley, M. Checovich, T. Havighurst, M. Schilling & W. B. Ershler
J Am Geriat Soc 43, 236-239 (1995)
^{ccxxi} S. H. Ralston
Analysis of gene expression in human bone biopsies by polymerase chain reaction: evidence for enhanced Cytokine expression in post-menopausal osteoporosis.
J Bone Miner Res 9, 883-890 (1994)
^{ccxxii} R. A. Daynes, B. A. Araneo, W. B. Ershler, C. Maloney, G. Z. Li & S. Y. Ryu
Altered regulation of IL-6 production with normal aging. Possible linkage to the age-associated decline in dehydroepiandrosterone and its sulfated derivative.
J Immunol 150, 5219-5230 (1993)
^{ccxxiii} R. Pacifici, C. Brown, E. Puscheck, E. Friedrich, E. Slatopolsky, D. Maggio, R. McCracken & L. V. Avioli
Effect of surgical menopause and estrogen replacement on Cytokine release from human blood mononuclear cells.
Proc Natl Acad Sci USA 88, 5134-5138 (1991)
^{ccxxiv} G. Pioli, G. Basini, M. Pedtazzoni, G. Musetti, V. Ulietti, D. Bresciani, P. Villa, A. Bacchi, D. Hughes, M. Russel & M. Passeri
Spontaneous release of interleukin-1 and interleukin-6 by peripheral blood mononuclear cells after oophorectomy.
Clin Sci 83, 503-507 (1992)
^{ccxxv} G. Girasole, R. L. Jilka, F. Passeri, S. Boswell, G. Boder, D. C. Williams & S. C. Manologas
17β-Estradiol inhibits interleukin-6 production by bone marrow-derived stromal cells and osteoblasts in vitro: a potential mechanism for the antiosteoporotic effect of estrogens.
J Clin Invest 89, 883-891 (1992)
^{ccxxvi} G. Girasole, R. L. Jilka, F. Passeri, S. Boswell, G. Boder, D. C. Williams & S. C. Manologas
17β-Estradiol inhibits interleukin-6 production by bone marrow-derived stromal cells and osteoblasts in vitro: a potential mechanism for the antiosteoporotic effect of estrogens)
J Clin Invest 89, 883-891 (1992
^{ccxxvii} R. L. Jilka, C. Hangoc, G. Girasole, G. Passeri, D. C. Williams, J. S. Abrams, B. Boyce, H. Broxmeyer & S. C. Manolagas
Increased osteoclast development after estrogen loss: Mediation by interleukin-6.
Science 257, 88-91 (1992)
^{ccxxviii} Omoigui S.
The Biochemical Origin of Pain: How a new law and new drugs have led to a medical breakthrough in the treatment of Persistent pain.
Hawthorne, Calif. State-of-the-Art Technologies Publishers 2004
^{ccxxix} Nakatsuka K.
Development of bisphosphonates
Nippon Rinsho. 2003 February; 61(2):219-25
^{ccxxx} Rodan G A, Reszka A A.
Bisphosphonate mechanism of action.
Curr Mol Med. 2002 September; 2(6):571-7.
^{ccxxxi} Reszka A A, Rodan G A.
Bisphosphonate mechanism of action.
Curr Rheumatol Rep. 2003 February; 5(1):65-74.
^{ccxxii} Bauer D C.
HMG CoA reductase inhibitors and the skeleton: a comprehensive review.
Osteoporos Int. 2003 June; 14(4):273-82. Epub 2003 May 8
^{ccxxiii} Funkhouser H L, Adera T, Adler R A
Effect of HMG-CoA reductase inhibitors (statins) on bone mineral density.
J Clin Densitom. 2002 Summer; 5(2): 151-8
^{ccxxiv} Pasco J A, Kotowicz M A, Henry M J, Sanders K M, Nicholson GC; Geelong Osteoporosis Study.
Statin use, bone mineral density, and fracture risk: Geelong Osteoporosis Study.
Arch Intern Med. 2002 Mar. 11; 162(5):537-40.
^{ccxxxv} Staal A, Frith J C, French M H, Swartz J, Gungor T, Harrity T W, Tamasi J, Rogers M J, Feyen J H
The ability of statins to inhibit bone resorption is directly related to their inhibitory effect on HMG-CoA reductase activity.
J Bone Miner Res. 2003 January; 18(1):88-96.
^{ccxxxvi} Harkness L S, Fiedler K, Sehgal A R, Oravec D, Lerner E
Decreased bone resorption with soy isoflavone supplementation in postmenopausal women.
J Womens Health (Larchmt). 2004 November; 13(9): 1000-7.
^{ccxxxvii} Lee Y B, Lee H J, Kim K S, Lee J Y, Nam S Y, Cheon S H, Sohn H S.
Evaluation of the preventive effect of isoflavone extract on bone loss in ovariectomized rats.
Biosci Biotechnol Biochem. 2004 May; 68(5):1040-5
^{ccxxxviii} Cotter A, Cashman K D.
Genistein appears to prevent early postmenopausal bone loss as effectively as hormone replacement therapy.
Nutr Rev. 2003 October; 61(10):346-51
^{ccxxxix} Horcajada-Molteni M N, Crespy V, Coxam V, Davicco M J, Remesy C, Barlet J P.
Rutin inhibits ovariectomy-induced osteopenia in rats.
J Bone Miner Res-2000 November; 15(11):2251-8.
^{ccxl} Sun D, Krishnan A, Zaman K, Lawrence R, Bhattacharya A, Fernandes G.
Dietary n-3 fatty acids decrease osteoclastogenesis and loss of bone mass in ovariectomized mice.
J Bone Miner Res. 2003 July; 18(7):1206-16.
^{ccxli} Kotake S, Sato K, Kim K J, Takahashi N, Udagawa N, Nakamura I, Yamaguchi A, Kishimoto T, Suda T, Kashiwazaki S.
Interleukin-6 and soluble interleukin-6 receptors in the synovial fluids from rheumatoid arthritis patients are responsible for osteoclast-like cell formation.
J Bone Miner Res. 1996 January; 11(1):88-95.
^{ccxlii}. F. Houssiau, J. P. Devoglaer, J. Van Damme, C. Nagant de Deuxchaisnes & J. Van Snick:
Interleukin-6 in synovial fluid and serum of patients with rheumatoid arthritis and other inflammatory arthritides.
Arthritis Rheum 31, 784-788 (1988)
^{ccxliii} Omoigui S.
The Biochemical Origin of Pain: How a new law and new drugs have led to a medical breakthrough in the treatment of Persistent pain.
Hawthorne, Calif. State-of-the-Art Technologies Publishers 2002
^{ccxliv} Olmos J M, De Vega T, Perera L, Riancho J A, Amado J A, Gonzalez-Macias J.
Departamento de Medicina Interna, Hospital Marques de Valdecilla, Universidad de Cantabria, Santander, Spain.
Etidronate inhibits the production of IL-6 by osteoblast-like cells.
Methods Find Exp Clin Pharmacol. 1999 October; 21(8):519-22.
^{ccxlv} Lissoni P, Cazzaniga M, Barni S, Perego M S, Brivio F, Fumagalli L, Tancini G.
Acute effects of Pamidronate administration on serum levels of interleukin-6 in advanced solid tumors with bone metastases and their possible implications in the immunotherapy of cancer with interleukin-2.
Eur J Cancer. 1997 February; 33(2):304-6.
^{ccxlvi} Abildgaard N, Rungby J, Glerup H, Brixen K, Kassem M, Brincker H, Heickendorff L, Eriksen E F, Nielsen J L.
Long-term oral pamidronate treatment inhibits osteoclastic bone resorption and bone turnover without affecting osteoblastic function in multiple myeloma.
Eur J Haematol. 1998 August; 61(2):128-34.
^{ccxlvii} Rendina D, Postiglione L, Vuotto P, Numis F G, Di Domenico G, Viceconti R, Mossetti G, Nunziata V.
Clodronate treatment reduces serum levels of interleukin-6 soluble receptor in Paget's disease of bone.
Clin Exp Rheumatol. 2002 May-June; 20(3):359-64.
^{ccxlviii} Giuliani N, Pedrazzoni M, Passeri G, Girasole G.
Bisphosphonates inhibit IL-6 production by human osteoblast-like cells.
Scand J Rheumatol. 1998; 27(1):38-41.
^{ccxlix} Vitte C, Fleisch H, Guenther H L.
Bisphosphonates induce osteoblasts to secrete an inhibitor of osteoclast-mediated resorption.
Endocrinology. 1996 June; 137(6):2324-33
^{ccl} Plotkin L I, Weinstein R S, Parfitt A M, Roberson P K, Manolagas S C, Bellido T.
Prevention of osteocyte and osteoblast apoptosis by bisphosphonates and calcitonin.
J Clin Invest. 1999 November; 104(10):1363-74.
^{ccli} Masuda-Aiba S, Shinozaki T, Takagishi K.
Effects of YM529, a novel minodronic acid, on adjuvant arthritis in rats.
Clin Exp Rheumatol. 2004 January-February; 22(1):71-8.
^{cclii} Thunyakitpisal P D, Chaisuparat R.
Simvastatin, an HMG-CoA reductase inhibitor, reduced the expression of matrix metalloproteinase-9 (Gelatinase B) in osteoblastic cells and HT1080 fibrosarcoma cells.
J Pharmacol Sci. 2004 April; 94(4):403-9
^{ccliii} Leung B P, Sattar N, Crilly A, Prach M, McCarey D W, Payne H, Madhok R, Campbell C,
Gracie J A, Liew F Y, McInnes I B.
A novel anti-inflammatory role for simvastatin in inflammatory arthritis.
J. Immunol. 2003 Feb. 1; 170(3):1524-30
^{ccliv} Diomede L, Albani D, Sottocorno M, Donati M B, Bianchi M, Fruscella P, Salmona M.
In vivo anti-inflammatory effect of statins is mediated by nonsterol mevalonate products.
Arterioscler Thromb Vasc Biol. 2001 August; 21(8):1327-32
^{cclv} Verdrengh M, Jonsson I M, Holmdahl R, Tarkowski A.
Genistein as an anti-inflammatory agent.
Inflamm Res. 2003 August; 52(8):341-6.
^{cclvi} Kuzuna S, Doi T, Morimoto S, Tsukuda R, Shino A, Maki Y.
[Effects of ipriflavone (TC-80, an anti-osteoporotic drug) on acute and chronic pain]
[Article in Japanese]
Nippon Yakurigaku Zasshi. 1986 July; 88(1):9-17
^{cclvii} Martinez-Dominguez E, de la Puerta R, Ruiz-Gutierrez V.
Protective effects upon experimental inflammation models of a polyphenol-supplemented virgin olive oil diet.
Inflamm Res 2001 February; 50(2):102-6.
^{cclviii} Haqqi T M, Anthony D D, Gupta S, Ahmad N, Lee M S, Kumar G K, Mukhtar H.
Prevention of collagen-induced arthritis in mice by a polyphenolic fraction from green tea.
Proc Natl Acad Sci USA. 1999 Apr. 13; 96(8):4524-9.
^{cclix} Cleland L G, James M J, Proudman S M.
The role of fish oils in the treatment of rheumatoid arthritis.
Drugs. 2003; 63(9):845-53
^{cclx} James M J, Proudman S M, Cleland L G.
Dietary n-3 fats as adjunctive therapy in a prototypic inflammatory disease: issues and obstacles for use in rheumatoid arthritis.
Prostaglandins Leukot Essent Fatty Acids. 2003 June; 68(6):399-405
^{cclxi} Adam O.
Dietary fatty acids and immune reactions in synovial tissue.
Eur J Med Res. 2003 Aug. 20; 8(8):381-7
^{cclxii} Yaffe K, Lindquist K, Penninx B W, Simonsick E M, Pahor M, Kritchevsky S, Launer L, Kuller L, Rubin S, Harris T.
Inflammatory markers and cognition in well-functioning African-American and white elders.
Neurology. 2003 Jul. 8; 61(1):76-80
^{cclxiii} J Nutr Health Aging. 2002; 6(5):324-31
HMG-CoA reductase inhibitors (statins) in the treatment of Alzheimer's disease and why it would be ill-advise to use one that crosses the blood-brain barrier.
Sparks D L, Connor D J, Browne P J, Lopez J E, Sabbagh M N.
^{cclxiv} J Mol Neurosci. 2004; 23(3):225-33
Apolipoprotein E as a target for developing new therapeutics for Alzheimer's disease based
^{on} studies from protein, RNA, and regulatory region of the gene.
Lahiri D K.
^{cclxv} Brain Res. 2002 Dec. 20; 958(1):100-11.
Secretion of apolipoprotein E by brain glia requires protein prenylation and is suppressed by statins.
Naidi A, Xu Q, Catalano R, Cordell B.
^{cclxvi} Eur J. Neurosci. 2003 January; 17(1):93-102.
Blockade of HMG-CoA reductase activity causes changes in microtubule-stabilizing protein tau via suppression of geranylgeranylpyrophosphate formation: implications for Alzheimer's disease.
Meske V, Albert F, Richter D, Schwarze J, Ohm T G.
^{cclxvii} Arch Neurol. 2002 March; 59(3):378-84.
Serum lipoprotein levels, statin use, and cognitive function in older women.
Yaffe K, Barrett-Connor E, Lin F, Grady D.
^{cclxviii} Arch Neurol. 2002 February; 59(2):223-7
Use of lipid-lowering agents, indication bias, and the risk of dementia in community-dwelling elderly people.
Rockwood K, Kirkland S, Hogan D B, MacKnight C, Merry H, Verreault R, Wolfson C, McDowell I.
^{cclxix} International Journal of Geriatric Psychiatry Volume 17, Issue 7, Pages 601-603
Primary hyperparathyroidism in an elderly woman: surgical reversibility of profound mental state problems due to mild hypercalcaemia
Ann McDonald C., David G. Bruce D G, Smith DJ
^{cclxx} Kim H, Xia H, Li L, Gewin J.
Attenuation of neurodegeneration-relevant modifications of brain proteins by dietary soy.
Biofactors. 2000; 12(1-4):243-50.
^{cclxxi} Kim C, Jang C H, Bang J H, Jung M W, Joo I, Kim S U, Mook-Jung I.
Amyloid precursor protein processing is separately regulated by protein kinase C and tyrosine kinase in human astrocytes.
Neurosci Left. 2002 May 24; 324(3): 185-8.
^{cclxxii} Ono K, Hasegawa K, Naiki H, Yamada M.
Anti-amyloidogenic activity of tannic acid and its activity to destabilize Alzheimer's beta-amyloid fibrin in vitro.
Biochim Biophys Acta. 2004 Nov. 5; 1690(3):193-202
^{cclxxiii} Rezai-Zadeh K, Shytle D, Sun N, Mori T, Hou H, Jeanniton D, Ehrhart J, Townsend K, Zeng J, Morgan D, Hardy J, Town T, Tan J.
Green tea epigallocatechin-3-gallate (EGCG) modulates amyloid precursor protein cleavage and reduces cerebral amyloidosis in Alzheimer transgenic mice.
J Neurosci. 2005 Sep. 21; 25(38):8807-14.
^{cclxxiv} Tully A M, Roche H M, Doyle R, Fallon C, Bruce I, Lawlor B, Coakley D, Gibney M J.
Low serum cholesteryl ester-docosahexaenoic acid levels in Alzheimer's disease: a case-control study.
Br J Nutr. 2003 April; 89(4):483-9
^{cclxxv} Puri B K, Bydder G M, Counsell S J, Corridan B J, Richardson A J, Hajnal J V, Appel C, Mckee H M, Vaddadi K S, Horrobin D F.
MRI and neuropsychological improvement in Huntington disease following ethyl-EPA treatment.
Neuroreport. 2002 Jan. 21; 13(1):123-6.
^{cclxxvi} Am J Physiol Regul Integr Comp Physiol 286: R1013-R1023, 2004
Interleukin-6 impairs endothelium-dependent NO-cGMP-mediated relaxation and enhances contraction in systemic vessels of pregnant rats
Orshal J. M., Khalil R. A.
^{cclxxvii} Weber J, Yang J C, Topalian S L, Parkinson D R, Schwartzentruber D S, Ettinghausen S E, Gunn H, Mixon A, Kim H, Cole D, et al.
Phase I trial of subcutaneous interleukin-6 in patients with advanced malignancies.
J Clin Oncol. 1993 March; 11 (3):499-506.
^{cclxxviii} Gaudino M, Andreotti F, Zamparelli R, Di Castelnuovo A, Nasso G, Burzotta F, Iacoviello L, Donati M B, Schiavello R, Maseri A, Possati G.
The −174G/C interleukin-6 polymorphism influences postoperative interleukin-6 levels and postoperative atrial fibrillation. Is atrial fibrillation an inflammatory complication?
Circulation. 2003 Sep. 9; 108 Suppl 1:II195-9
^{cclxxix} J Hum Hypertens. 2004 Sep. 9
Long-term effects of statins on arterial pressure and stiffness of hypertensives.
Ichihara A, Hayashi M, Koura Y, Tada Y, Kaneshiro Y, Saruta T.
^{cclxxx} Am Heart J. 2004 August; 148(2):285-92.
Association between different lipid-lowering treatment strategies and blood pressure control in the Brisighella Heart Study.
Borghi C, Dormi A, Veronesi M, Sangiorgi Z, Gaddi A; Brisighella Heart Study Working Party.
^{cclxxxi} Amar D, Zhang H, Heerdt P M, Park B, Fleisher M, Thaler H T.
Statin use is associated with a reduction in atrial fibrillation after noncardiac thoracic surgery independent of C-reactive protein.
Chest. 2005 November; 128(5):3421-7.
^{cclxxxii} Kitayama J, Kitazono T, Ooboshi H, Ago T, Ohgami T, Fujishima M, Ibayashi S.
Chronic administration of a tyrosine kinase inhibitor restores functional and morphological changes of the basilar artery during chronic hypertension.
J Hypertens. 2002 November; 20(11):2205-11.
^{cclxxxiii} Isoflavones from red clover improve systemic arterial compliance but not plasma lipids in menopausal women.
J Clin Endocrinol Metab. 1999 March; 84(3):895-8.
Nestel P J, Pomeroy S, Kay S, Komesaroff P, Behrsing J, Cameron J D, West L.
^{cclxxxiv} Negishi H, Xu J W, Ikeda K, Njelekela M, Nara Y, Yamori Y.
Black and green tea polyphenols attenuate blood pressure increases in stroke-prone spontaneously hypertensive rats.
J Nutr. 2004 January; 134(1):38-42.
^{cclxxxv} Bonaa K H, Bjerve K S, Straume B, Gram I T, Thelle D.
Effect of eicosapentaenoic and docosahexaenoic acids on blood pressure in hypertension. A population-based intervention trial from the Tromso study.
N Engl J Med. 1990 Mar. 22; 322(12):795-801.
^{cclxxxvi} Engler M M, Engler M B, Pierson D M, Molteni L B, Molteni A.
Effects of docosahexaenoic acid on vascular pathology and reactivity in hypertension.
Exp Biol Med (Maywood). 2003 March; 228(3):299-307.
^{cclxxxvii} Aneja R, Hake P W, Burroughs T J, Denenberg A G, Wong H R, Zingarelli B. Epigallocatechin, a green tea polyphenol, attenuates myocardial ischemia reperfusion injury in rats.
Mol Med. 2004 January-June; 10(1-6):55-62.
^{cclxxxviii} Matthan N R, Jordan H, Chung M, Lichtenstein A H, Lathrop D A, Lau J.
A systematic review and meta-analysis of the impact of omega-3 fatty acids on selected arrhythmia outcomes in animal models.
Metabolism. 2005 December; 54(12): 1557-65.
^{cclxxxix} Mozaffarian D, Lemaitre R N, Kuller L H, Burke G L, Tracy R P, Siscovick D S; Cardiovascular Health Study.
Cardiac benefits of fish consumption may depend on the type of fish meal consumed: the Cardiovascular Health Study.
Circulation 2003 Mar. 18; 107(10):1372-7.
^{ccxc} Schlesinger T K, Bonvin C, Jarpe M B, Fanger G R, Cardinaux J R, Johnson G L, Widmann C.
Apoptosis stimulated by the 91-kDa caspase cleavage MEKK1 fragment requires translocation to soluble cellular compartments.
J Biol Chem. 2002 Mar. 22; 277(12):10283-91. Epub 2002 Jan. 8.
^{ccxci} Duncan R E, El-Sohemy A, Archer M C.
Dietary factors and the regulation of 3-hydroxy-3-methylglutaryl coenzyme A reductase: implications for breast cancer and development.
Mol Nutr Food Res 2005 February; 49(2):93-100
^{ccxcii} Mitchell T J, John S.
Signal transducer and activator of transcription (STAT) signalling and T-cell lymphomas.
Immunology. 2005 March; 114(3):301-12.
^{ccxciii} Xu Q Briggs J, Park S, Niu G, Kortylewski M, Zhang S, Gritsko T, Turkson J, Kay H, Semenza G L, Cheng J Q, Jove R, Yu H.
Targeting Stat3 blocks both HIF-1 and VEGF expression induced by multiple oncogenic growth signaling pathways.
Oncogene. 2005 Aug. 25; 24(36):5552-60.
^{ccxciv} Barton B E, Karras J G, Murphy T F, Barton A, Huang H F.
Signal transducer and activator of transcription 3 (STAT3) activation in prostate cancer: Direct STAT3 inhibition induces apoptosis in prostate cancer lines.
Mol Cancer Ther 2004 January; 3(1):11-20
^{ccxcv} Cote S, Lemieux R, Simard C.
The survival of IL-6-dependent myeloma cells critically relies on their capability to transit the G1 to S phase interval of the cell cycle.
Cell Signal. 2005 May; 17(5):615-24
^{ccxcvi} Weissenberger J, Loeffler S, Kappeler A, Kopf M, Lukes A, Afanasieva T A, Aguzzi A, Weis J.
IL-6 is required for glioma development in a mouse model.
Oncogene. 2004 Apr. 22; 23(19):3308-16.
^{ccxcvii} Leu C M, Wong F H, Chang C, Huang S F, Hu C P.
Interleukin-6 acts as an antiapoptotic factor in human esophageal carcinoma cells through the activation of both STAT3 and mitogen-activated protein kinase pathways.
Oncogene. 2003 Oct. 30; 22(49):7809-18.
^{ccxcviii} Sanchez A, Nagy P, Thorgeirsson S S.
STAT-3 activity in chemically-induced hepatocellular carcinoma.
Eur J Cancer. 2003 September; 39(14):2093-8.
^{ccxcix} Chen C Y, Tsay W, Tang J L, Shen H L, Lin S W, Huang S Y, Yao M, Chen Y C, Shen M C, Wang C H, Tien H F.
SOCS1 methylation in patients with newly diagnosed acute myeloid leukemia.
Genes Chromosomes Cancer. 2003 July; 37(3):300-5
^{ccc} Galm O. Yoshikawa H, Esteller M, Osieka R, Herman J G.
SOCS-1, a negative regulator of cytokine signaling, is frequently silenced by methylation in multiple myeloma.
Blood. 2003 Apr. 1; 101(7):2784-8. Epub 2002 Nov. 27.
^{ccci} Barton B E
Interleukin-6 and new strategies for the treatment of cancer, hyperproliferative diseases and paraneoplastic syndromes
Expert Opin Ther Targets. 2005 August; 9(4):737-52
^{ccci} Bharti A C, Shishodia S, Reuben J M, Weber D, Alexanian R, Raj-Vadhan S, Estrov Z, Talpaz M, Aggarwal B B.
Nuclear factor-kappaB and STAT3 are constitutively active in CD138+ cells derived from multiple myeloma patients, and suppression of these transcription factors leads to apoptosis.
Blood. 2004 Apr. 15; 103(8):3175-84. Epub 2003 Dec. 18.
^{ccci} Bharti A C, Donato N, Singh S, Aggarwal B B.
Curcumin (diferuloylmethane) down-regulates the constitutive activation of nuclear factor-kappa B and IkappaBalpha kinase in human multiple myeloma cells, leading to suppression of proliferation and induction of apoptosis.
Blood. 2003 Feb. 1; 101(3):1053-62. Epub 2002 Sep. 5.
^{ccciv} Culig Z, Steiner H, Bartsch G, Hobisch A.
J Cell Biochem. 2005 Jun. 1; 95(3):497-505.
Interleukin-6 regulation of prostate cancer cell growth.
^{cccv} Tan D, Wu X, Hou M, Lee S O, Lou W, Wang J, Janarthan B, Nallapareddy S, Trump D L, Gao A C.
Interleukin-6 polymorphism is associated with more aggressive prostate cancer.
J Urol. 2005 August; 174(₂):753-6.
^{cccvi} Gordon A H, O'Keefe R J, Schwarz E M, Rosier R N, Puzas J E.
Nuclear factor-kappaB-dependent mechanisms in breast cancer cells regulate tumor burden and osteolysis in bone.
Cancer Res. 2005 Apr. 15; 65(8):3209-177
^{cccvii} Soubrane C, Rixe O, Meric J B, Khayat D, Mouawad R.
Pretreatment serum interleukin-6 concentration as a prognostic factor of overall survival in metastatic malignant melanoma patients treated with biochemotherapy: a retrospective study.
Melanoma Res. 2005 June; 15(3): 199-204
^{cccviii} Costanzo E S, Lutgendorf S K, Sood A K, Anderson B, Sorosky J, Lubaroff D M.
Psychosocial factors and interleukin-6 among women with advanced ovarian cancer.
Cancer. 2005 Jul. 15; 104(2):305-13.
^{cccix} Kai H, Kitadai Y, Kodama M, Cho S, Kuroda T, Ito M, Tanaka S, Ohmoto Y, Chayama K.
Involvement of proinflammatory cytokines IL-1beta and IL-6 in progression of human gastric carcinoma.
Anticancer Res. 2005 March-April; 25 (2A):709-13.
^{cccx} Alberti L, Thomachot M C, Bachelot T, Menetrier-Caux C, Puisieux I, Blay J Y.
IL-6 as an intracrine growth factor for renal carcinoma cell lines.
Int J Cancer. 2004 Sep. 20; 111(5):653-61.
^{cccxl} Su J L, Lai K P, Chen C A, Yang C Y, Chen P S, Chang C C, Chou C H, Hu C L, Kuo M L, Hsieh C Y, Wei L H.
A novel peptide specifically binding to interleukin-6 receptor (gp80) inhibits angiogenesis and tumor growth.
Cancer Res. 2005 Jun. 1; 65(11):4827-35.
^{cccxii} Illman J, Corringham R, Robinson D Jr, Davis H M, Rossi J F, Cella D, Trikha M.
Are inflammatory cytokines the common link between cancer-associated cachexia and depression?
J Support Oncol. 2005 January-February; 3(1):37-50.
^{cccxiii} Wu J, Wong W W, Khosravi F, Minden M D, Penn L Z.
Blocking the Raf/MEK/ERK pathway sensitizes acute myelogenous leukemia cells to lovastatin-induced apoptosis.
Cancer Res. 2004 Sep. 15; 64(18):6461-8.
^{cccxiv} Cafforio P, Dammacco F, Gernone A, Silvestris F.
Statins activate the mitochondrial pathway of apoptosis in human lymphoblasts and myeloma cells.
Carcinogenesis. 2005 May; 26(5):883-91. Epub 2005 Feb. 10.
^{cccxv} Muck A O, Seeger H, Wallwiener D.
Inhibitory effect of statins on the proliferation of human breast cancer cells.
Int J Clin Pharmacol Ther. 2004 December; 42(12):695-700.
^{cccxvi} Horiguchi A, Sumitomo M, Asakuma J, Asano T, Asano T, Hayakawa M.
3-hydroxy-3-methylglutaryl-coenzyme a reductase inhibitor, fluvastatin, as a novel agent for prophylaxis of renal cancer metastasis.
Clin Cancer Rest 2004 Dec. 15; 10(24):8648-55.
^{cccxvii} Poynter J N, Gruber S B, Higgins P D, Almog R, Bonner J D, Rennert H S, Low M,
Statins and the risk of colorectal cancer.
N Engl J Med. 2005 May 26; 352(21):2184-92.
^{cccxviii} cyrus-David M S, Weinberg A, Thompson T, Kadmon D.
The effect of statins on serum prostate specific antigen levels in a cohort of airline pilots: a preliminary report.
J Urol. 2005 June; 173(₆):1923-5
^{cccxix} Caraglia M, D'Alessandro A M, Marra M, Giuberti G, Vitale G, Viscomi C, Colao A, Prete S D, Tagliaferri P, Tassone P, Budillon A, Venuta S, Abbruzzese A.
The farnesyl transferase inhibitor R115777 (Zarnestra) synergistically enhances growth inhibition and apoptosis induced on epidermoid cancer cells by Zoledronic acid (Zometa) and Pamidronate.
Oncogene. 2004 Sep. 9; 23(41):6900-13
^{cccxx} Muller S, Migianu E, Lecouvey M, Kraemer M, Oudar O
Alendronate inhibits proliferation and invasion of human epidermoid carcinoma cells in vitro.
Anticancer Res. 2005 July-August; 25(4):2655-60.
^{cccxxi} Green J R.
Skeletal complications of prostate cancer: pathophysiology and therapeutic potential of bisphosphonates.
Acta Oncol. 2005; 44(3):282-92.
^{cccxxii} Segawa H, Kimura S, Kuroda J, Sato K, Nogawa M, Yuasa T, Yokota A, Hodohara K, Fujiyama Y, Maekawa T.
The anti-leukemic efficacy of the third generation bisphosphonate ONO5920/YM529.
Leuk Res. 2005 April; 29(4):451-7
^{cccxxiii} Radzikowski C, Wietrzyk J, Grynkiewicz G, Opolski A.
[Genistein: a soy isoflavone revealing a pleiotropic mechanism of action—clinical implications in the treatment and prevention of cancer]
[Article in Polish]
Postepy Hig Med Dosw (Online). 2004 Feb. 27; 58:128-39
^{cccxxiv} Zhang M, Xie X, Lee A H, Binns C W.
Soy and isoflavone intake are associated with reduced risk of ovarian cancer in southeast china.
Nutr Cancer. 2004; 49(2): 125-30.
^{cccxxv} Sarkar F H, Li Y.
Comment in: Cancer Invest. 2003; 21(5):817-8.
Soy isoflavones and cancer prevention.
Cancer Invest. 2003; 21(5):744-57.
^{cccxxvi} Park O J, Surh Y J.
Chemopreventive potential of epigallocatechin gallate and genistein: evidence from epidemiological and laboratory studies.
Toxicol Lett. 2004 Apr. 15; 150(1):43-56.
^{cccxxvii} Shimizu M, Weinstein I B.
Modulation of signal transduction by tea catechins and related phytochemicals.
Mutat Res. 2005 Jun. 28; [Epub ahead of print
^{cccxxviii} Bektic J, Guggenberger R, Eder I E, Pelzer A E, Berger A P, Bartsch G, Klocker H.
Molecular effects of the isoflavonoid genistein in prostate cancer
Clin Prostate Cancer. 2005 September; 4(2): 124-9
^{cccxxix} Baxa D M, Yoshimura F K.
Genistein reduces NF-kappa B in T lymphoma cells via a caspase-mediated cleavage of I kappa B alpha.
Biochem Pharmacol. 2003 Sep. 15; 66(6):1009-18.
^{cccxxx} Ravindranath M H, Muthugounder S, Presser N, Viswanathan S.
Anticancer therapeutic potential of soy isoflavone, genistein.
Adv Exp Med Biol. 2004; 546:121-65
^{cccxxxi} Liu Y, Zhang Y M, Song D F, Cui H B.
[Effect of apoptosis in human breast cancer cells and its probable mechanisms by genistein]
[Article in Chinese]
Wei Sheng Yan Jiu. 2005 January; 34(1):67-9
^{cccxxxii} Hiliman G G, Wang Y, Kucuk O, Che M, Doerge D R, Yudelev M, Joiner M C, Marples B, Forman J D, Sarkar F H
Genistein potentiates inhibition of tumor growth by radiation in a prostate cancer orthotopic model.
Mol Cancer Ther. 2004 October; 3(10):1271-9
^{cccxxxiii} Farivar R S, Gardner-Thorpe J, Ito H, Arshad H, Zinner M J, Ashley S W, Whang E E.
The efficacy of tyrosine kinase inhibitors on human pancreatic cancer cell lines
J Surg Res. 2003 December; 115(2):219-25*
^{cccxxxiv} Wang Y, Zhang Y T, Liu F, Zhang S, Wang W, Li Y M.
[Effect of genistein on proliferation of renal cell carcinoma cell line GRC-1 and its influence to p27 expression]
[Article in Chinese]
Ai Zheng. 2003 December; 22(12): 1272-5-
^{cccxxxv} Baliga M S, Meleth S, Katiyar S K.
Growth inhibitory and antimetastatic effect of green tea polyphenols on metastasis-specific mouse mammary carcinoma 4T1 cells in vitro and in vivo systems.
Clin Cancer Res. 2005 Mar. 1; 11(5): 1918-27
^{cccxxxvi} Pianetti S, Guo S, Kavanagh K T, Sonenshein G E.
Green tea polyphenol epigallocatechin-3 gallate inhibits Her-2/neu signaling, proliferation, and transformed phenotype of breast cancer cells.
Cancer Res. 2002 Feb. 1; 62(3):652-5
^{cccxxxvii} Ahmad N, Gupta S, Mukhtar H.
Green tea polyphenol epigallocatechin-3-gallate differentially modulates nuclear factor kappaB in cancer cells versus normal cells.
Arch Biochem Biophys 2000 Apr. 15; 376(2):338-46
^{cccxxxviii} Dong Z.
Effects of food factors on signal transduction pathways.
Biofactors. 2000; 12(1-4): 17-28.
^{cccxxxix} J Nutr 2004 December; 134(12 Suppl):3427S-3430S.
(n-3) fatty acids and cancer therapy.
J Nutr. 2004 December; 134(12 Suppl):3427S-3430S.
^{cccxl} Sharma A, Bena J, Logan J, Espat J, Hurteau J A.
The effects of Omega-3 fatty acids on growth regulation of epithelial ovarian cancer cell lines.
Gynecol Oncol. 2005 October; 99(1):58-64.
^{cccxli} Caruso C, Lio D, Cavallone L, Franceschi C.
Aging, longevity, inflammation, and cancer.
Ann N Y Acad Sci 2004 December; 1028:1-13.
^{cccxlii} Wu K D, Orme L M, Shaughnessy J Jr, Jacobson J, Barlogie B, Moore M A.
Telomerase and telomere length in multiple myeloma: correlations with disease heterogeneity, cytogenetic status, and overall survival.
Bllod. 2003 Jun. 15; 101(12):4982-9. Epub 2003 Feb. 27.
^{cccxliii} Kiecolt-Glaser J K, Preacher K J, MacCallum R C, Atkinson C, Malarkey W B, Glaser R.
Chronic stress and age-related increases in the proinflammatory cytokine IL-6.
Proc Natl Acad Sci USA. 2003 Jul. 22; 100(15):9090-5. Epub 2003 Jul. 2.
^{cccxliv} Clegg K B, Sambhi M P.
Inhibition of epidermal growth factor-mediated DNA synthesis by a specific tyrosine kinase inhibitor in vascular smooth muscle cells of the spontaneously hypertensive rat.
J Hypertens Suppl. 1989 December; 7(6): S144-5-
^{cccxlv} Urbanski A, Schwarz T, Neuner P, Krutmann J, Kimbauer R, Kock A, Luger T A.
Ultraviolet light induces increased circulating interleukin-6 in humans.
J Invest Dermatol. 1990 June; 94(6):808-11.
^{cccxlvi} Xia J, Song X, Bi Z, Chu W, Wan Y.
UV-induced NF-kappaB activation and expression of IL-6 is attenuated by (−)-epigallocatechin-3-gallate in cultured human keratinocytes in vitro.
Int J Mol Med. 2005 November; 16(5):943-50.
^{cccxlvii} Seddon J M, George S, Rosner B, Rifai N.
Progression of age-related macular degeneration: prospective assessment of C-reactive protein, interleukin 6, and other cardiovascular biomarkers.
Arch Ophthalmol 2005 June; 123(6):774-82
^{cccxlviii} Fishman D, Faulds G, Jeffery R, Mohamed-Ali V, Yudkin J S, Humphries S, Woo P:
The effect of novel polymorphisms in the interleukin-6 (IL-6) gene on IL-6 transcription and plasma IL-6 levels, and an association with systemic-onset juvenile chronic arthritis. J Clin Invest 102 :1369-1376, 1998
^{cccxlix} Linker-Israeli, M., D. J. Wallace, J. L. Prehn, R. Nand, L. Li, and J. R. Klinenberg (1996)
A greater variability in the 3′ flanking region of the IL-6 gene in patients with systemic lupus erythematosus (SLE). Autoimmunity. 23: 199-209
^{cccl} Murray, R., F. McGuigan, S. Grant, D. Reid, and S. Ralston (1997) Polymorphisms of the interleukin-6 gene are associated with bone mineral density. Bone. 21: 89-92
^{cccli} Cannon C P, et al. (2004). Comparison of intensive and moderate lipid lowering with statins after acute coronary syndromes. New England Journal of Medicine, 350: 1-10
^{ccclii} Nissen S E, et al. (2004). Effect of intensive compared with moderate lipid-lowering therapy on progression of coronary atherosclerosis. JAMA, 291(9): 1071-1080
^{ccciiii} Downs J R, et al. (1998). Primary prevention of acute coronary events with lovastatin in
^{men} and women with average cholesterol levels: Results of AFCAPS/TexCAPS. JAMA, 279(20): 1615-1622.
^{cccliv} Pignone M P, et al. (2001). Screening and treating adults for lipid disorders. American Journal of Preventive Medicine, 20(3S): 77-89.
^{ccclv} Long-Term Intervention with Pravastatin in Ischaemic Disease (LIPID) Study Group (1998). Prevention of cardiovascular events and death with pravastatin in patients with coronary heart disease and a broad range of initial cholesterol levels. New England Journal of
Medicine, 339(19): 1349-1357-
^{ccclvi} Scandinavian Simvastatin Survival Study Group (1994). Randomised trial of cholesterol lowering in 4,444 patients with coronary heart disease: The Scandinavian Simvastatin Survival Study (4S). Lancet, 344(8934): 1383-1389
^{ccclvii} Shepherd J, et al. (2002). Pravastatin in elderly individuals at risk of vascular disease (PROSPER): A randomised controlled trial. Lancet, 360(9346): 1623-1630
^{ccclviii} LaRosa J C, et al. (1999). Effect of statins on risk of coronary disease: A meta-analysis
^{of} randomized controlled trials. JAMA, 282(24): 2340-2346.
^{ccclix} Sudlow C, et al. (2003). Secondary prevention of ischaemic cardiac events. Clinical Evidence, (9): 166-205.
^{ccclx} Curr Atheroscler Rep. 2004 September; 6(5):366-74
Lessons learned from the prospective pravastatin pooling project.
Byington R P, Sacks F M.
^{ccclxi} Atheroscler Suppl. 2003 December; 4(5):11-6
Long-term statin safety and efficacy in secondary prevention: can combination therapy improve outcomes?
Wascher T C
^{ccclxii} Int J Clin Pract. 2005 January; 59(1):121-3.
The collaborative atorvastatin diabetes study: preliminary results.
Owen O G.
^{ccclxiii} Expert Opin Drug Saf. 2002 September; 1(3):269-74
A safety look at currently available statins.
Moghadasian M H
^{ccclxiv} Drug Saf. 2003; 26(9):661-71.
Extended safety profile of oral clodronate after long-term use in primary breast cancer patients.
Atula S, Powles T, Paterson A, McCloskey E, Nevalainen J, Kanis J
^{ccclxv} Bone. 2002 November; 31(5):620-5
Effects of long-term risedronate on bone quality and bone turnover in women with postmenopausal osteoporosis.
Eriksen E F, Melsen F, Sod E, Barton I, Chines A.
^{ccclxvi} Black D M, Cummings S R, Karpf D B, Cauley J A, Thompson D E, Nevitt M C, et al.
Randomised trial of effect of alendronate on risk of fracture in women with existing vertebral fractures. Fracture Intervention Trial Research Group. Lancet 1996; 348:1535-41
^{ccclxvii} Cummings S R, Black D M, Thompson D E, Applegate W B, Barrett-Connor E, Musliner T A, et al. Effect of alendronate on risk of fracture in women with low bone density but without vertebral fractures: results from the Fracture Intervention Trial. JAMA 1998; 280:2077-82.
^{ccclxviii} Am Fam Physician. 2000 May 1; 61(9):2731-6
Comment in:
Am Fam Physician. 2001 May 15; 63(10):1913-4, 1916.
Bisphosphonates: safety and efficacy in the treatment and prevention of osteoporosis.
Greenspan S L, Harris S T, Bone H, Miller P D, Orwoll E S, Watts N B, Rosen C J.

1-96. (canceled)

97. A method for treating an age-related disorder in a human or animal subject comprising administering to said subject an effective amount of an anti-Interleukin-6 (IL-6) anti-inflammatory agent that inhibits IL-6-mediated inflammation in said subject.

98. The method of claim 97, wherein said anti-IL-6 anti-inflammatory agent inhibits IL-6-mediated inflammation in said subject by modulating endogenous cholesterol synthesis in the subject.

99. The method of claim 97, wherein said anti-IL-6 anti-inflammatory agent inhibits IL-6-mediated inflammation in said subject by modulating endogenous isoprenoid depletion in the subject.

100. The method of claim 99, wherein said anti-IL-6 anti-inflammatory agent inhibits IL-6-mediated inflammation in said subject by inhibiting cholesterol synthesis in the subject.

101. The method of claim 100, wherein said anti-IL-6 anti-inflammatory agent that inhibits cholesterol synthesis in the subject is selected from statins, and bisphosphonates.

102. The method of claim 100, wherein said anti-IL-6 anti-inflammatory agent that inhibits cholesterol synthesis in the subject is a statin or statin-containing composition selected from the group consisting of lovastatin, simvastatin, pravastatin, fluvastatin, atorvastatin, rivastatin, red yeast rice, red yeast grain, red yeast powder, and fermentation products of a filamentous fungi.

103. The method of claim 100, wherein said anti-IL-6 anti-inflammatory agent that inhibits cholesterol synthesis in the subject is a bisphosphonate selected from the group consisting of pamidronate, etidronate, clodronate, amdalendronate, and phosphonic acid derivatives and esters thereof.

104. The method of claim 98, wherein said anti-IL-6 anti-inflammatory agent that inhibits IL-6-mediated inflammation is a cholesterol lowering agent.

105. The method of claim 104, wherein said cholesterol lowering agent is selected from the group consisting of (i) cholesterol sequestrants, (ii) nicotinyl alcohol, nicotinic acid, and salts thereof, (iii) PPAR alpha agonists, (iv) inhibitors of cholesterol absorption, (v) phytosterols and phytosterol-containing food products, and (vi) acyl CoA:cholesterol acyltransferase inhibitors.

106. The method of claim 97, wherein said anti-IL-6 anti-inflammatory agent inhibits IL-6-mediated inflammation in said subject by inhibiting IL-6 expression, activity, and/or signal transduction in the subject.

107. The method of claim 106, wherein said anti-IL-6 anti-inflammatory agent is an anti-IL6 antibody or inhibitor that binds, neutralizes, and/or inhibits a signal transduction activity of IL-6, or an anti-IL6 receptor antibody or inhibitor that binds, neutralizes, and/or inhibits a signal transduction activity of an IL-6 receptor.

108. The method of claim 106, wherein said anti-IL-6 anti-inflammatory agent is an antisense oligonucleotide (ASON) that inhibits IL-6 expression in said subject.

109. The method of claim 106, wherein said anti-IL-6 anti-inflammatory agent is an antibody or inhibitor directed against one or more endogenous targets involved in IL-6 signal transduction and/or metabolism selected from gp130 protein, tyrosine kinases, serine/threonine kinases, mitogen-activated protein (MAP) kinases, phosphatidylinositol 3-kinase (PI3K), Nuclear factor κB (NF-κB), IκB kinase (IKK), activator protein-1 (AP-1), and STAT transcription factors.

110. The method of claim 109, wherein said anti-IL-6 anti-inflammatory agent is selected from suppressors of cytokine signaling (SOCS) proteins, activators and ligands of PPAR gamma and PPAR beta/delta, and functional fragments thereof.

111. The method of claim 109, wherein said anti-IL-6 anti-inflammatory agent is selected from the group consisting of inactive and active forms of vitamin D, 1-alpha, 25-dihydroxyvitamin D3 (1-alpha, 25 (OH)2D3) and its analogs; synthetic or plant derived polyphenolic compounds including phenolic acids, flavonoids, stilbenes, lignans, and anthocyanidins; flavanols; flavones; flavonols, flavanones, isoflavones and their naturally occurring glycosides; proanthocyanidins and their gallic acid esters; soy protein material and their naturally occurring glycosides; soy-based food products and soybean-derived protein ingredients; Cocoa polyphenols and derivatives thereof; polyphenols found in nuts, nut skin extracts, tea and derivatives thereof; polyphenols of vegetables and fruits; polyphenols derived from fruits, vegetables, cereals, dry legumes, chocolate, and beverages including grape juice tea, coffee, or wine; and Omega 3 fatty acids.

112. The method of claim 106, wherein said anti-IL-6 anti-inflammatory agent comprises an altered IL-6, a partial peptide of IL-6, or an IL-6 receptor protein or peptide.

113. The method of claim 112, wherein said IL-6 or IL-6 receptor protein or peptide is artificially modified by a recombinant or chemical alteration.

114. The method of claim 97, wherein said age-related disorder is atherosclerosis.

115. The method of claim 97, wherein said age-related disorder is peripheral vascular disease.

116. The method of claim 97, wherein said age-related disorder is coronary artery disease.

117. The method of claim 97, wherein said age-related disorder is arrhythmia.

118. The method of claim 97, wherein said age-related disorder is hypertension

119. The method of claim 97, wherein said age-related disorder is osteoporosis

120. The method of claim 97, wherein said age-related disorder is arthritis

121. The method of claim 97, wherein said age-related disorder is type 1 diabetes, type 2 diabetes, inadequate glucose tolerance or insulin resistance.

122. The method of claim 97, wherein said age-related disorder is obesity.

123. The method of claim 97, wherein said age-related disorder is dementia

124. The method of claim 97, wherein said age-related disorder is Alzheimer's disease Huntington's disease, or Parkinson's disease.

125. The method of claim 97, wherein said age-related disorder is skin wrinkles, sunburn, or skin damage.

126. The method of claim 97, wherein said age-related disorder is age-related macular degeneration (AMD).

127. The method of claim 97, wherein said age-related disorder is periodontal disease.

128. The method of claim 97, wherein said age-related disorder is a chronic low grade infection.

129. The method of claim 97, wherein said age-related disorder is a primary or secondary cancer or tumor selected from the group consisting of adrenal cancer, astrocytoma, basal or squamous cell carcinoma, brain cancer, bladder cancer, breast cancer, colorectal cancer, chrondrosarcoma, cervical cancer, choriocarcinoma, esophageal cancer, endometrial carcinoma, erythroleukemia, Ewing's sarcoma, gastrointestinal cancer, gliobastoma, glioma, head and neck cancer, hepatocellular carcinoma, hepatoma, leiomyoma, leukemia, melanoma, multiple myeloma, neural cancer, lung cancer, osteosarcoma, ovarian cancer, pancreatic cancer, prostate cancer, renal cell carcinoma, rhabdomyosarcoma, small cell lung cancer, testicular cancer, and thyroid cancer.

130. The method of claim 97, wherein said anti-Interleukin-6 (IL-6)-inflammatory agent is administered to said subject subcutaneously, intramuscularly, intravenously, orally, rectally, sublingually, transmucosally, inhalationally, or transdermally.

131. The method of claim 97, wherein said anti-IL-6 anti-inflammatory agent is administered to said subject by gene therapy or gene modulation.

132. The method of claim 131, wherein said anti-IL-6 anti-inflammatory agent is an altered IL-6 gene modified by a base change of guanine to cytosine at position 174 in a 5′ flanking region of the interleukin-6 gene.

133. The method of claim 131, wherein said anti-IL-6 anti-inflammatory agent is an altered IL-6 gene administered to a cell as naked DNA.

134. The method of claim 131, wherein said anti-IL-6 anti-inflammatory agent is an altered IL-6 gene administered to a cell in a vector.

135. The method of claim 134, wherein said vector comprises a promoter or regulator to control expression of the interleukin-6 gene.

136. The method of claim 134, wherein said vector comprises targeting means to target the vector to a particular tissue or cell type.

137. The method of claim 134, wherein said vector comprises a viral vector.

138. The method of claim 134, wherein said vector is derived from a DNA virus, an RNA virus, or a retrovirus.

139. The method of claim 134, wherein said vector comprises an adenovirus vector.

140. The method of claim 131, wherein gene modulation of an interleukin-6 gene is achieved by introducing to said subject an antisense gene or gene fragment, or by introducing to said subject a vector that produce a ribozyme that cleaves a specific mRNA.

141. The method of claim 131, wherein said gene modulation is achieved by introduction of an exogenous normal genetic sequence that codes for a protein that modulates gene expression or affects processing, assembly, or secretion of a gene product.
